SEQUENCE LISTING (62 sequences)

SEQ ID NO: 1 - protein, 9 aa, HUMAN
RPPGFSPFR

SEQ ID NO: 2 - protein, 10 aa, HUMAN
KRPPGFSPFR

SEQ ID NO: 3 - protein, 11 aa, ARTIFICIAL
  SYNTHETICALLY PRODUCED
MKRPPGFSPFR

SEQ ID NO: 4 - protein, 8 aa, HUMAN
RPPGFSPF

SEQ ID NO: 5 - protein, 8 aa, ARTIFICIAL
  SYNTHETICALLY PRODUCED
RPPGFSPL

SEQ ID NO: 6 - protein, 9 aa, ARTIFICIAL
  SYNTHETICALLY PRODUCED
KRPPGFSPL

SEQ ID NO: 7 - protein, 10 aa, ARTIFICIAL
  SYNTHETICALLY PRODUCED
XRPXGXSXXR

SEQ ID NO: 8 - protein, 9 aa, ARTIFICIAL
  SYNTHETICALLY PRODUCED
XRPXGXSXX

SEQ ID NO: 9 - protein, 10 aa, ARTIFICIAL
  SYNTHETICALLY PRODUCED
XRPXGXSXXR

SEQ ID NO: 10 - protein, 10 aa, ARTIFICIAL
  SYNTHETICALLY PRODUCED
LLRPPGXSXI

SEQ ID NO: 11 - protein, 10 aa, ARTIFICIAL
  SYNTHETICALLY PRODUCED
XRPXGXSXXR

SEQ ID NO: 12 - protein, 10 aa, ARTIFICIAL
  SYNTHETICALLY PRODUCED
KKRPXGXSXX

SEQ ID NO: 13 - protein, 10 aa, ARTIFICIAL
  SYNTHETICALLY PRODUCED
KKRPXGXSXX

SEQ ID NO: 14 - protein, 10 aa, ARTIFICIAL
  SYNTHETICALLY PRODUCED
XRPXGXSXXR

SEQ ID NO: 15 - protein, 10 aa, ARTIFICIAL
  SYNTHETICALLY PRODUCED
XLRPXGXSXX

SEQ ID NO: 16 - protein, 10 aa, ARTIFICIAL
  SYNTHETICALLY PRODUCED
XLRPXGXSXX

SEQ ID NO: 17 - protein, 10 aa, ARTIFICIAL
  SYNTHETICALLY PRODUCED
XLRPXGXSXX

SEQ ID NO: 18 - protein, 10 aa, ARTIFICIAL
  SYNTHETICALLY PRODUCED
XLRPXGXSXX

SEQ ID NO: 19 - protein, 9 aa, ARTIFICIAL
  SYNTHETICALLY PRODUCED
XRPXGXSXX

SEQ ID NO: 20 - protein, 9 aa, ARTIFICIAL
  SYNTHETICALLY PRODUCED
XRPXGXSXX

SEQ ID NO: 21 - protein, 9 aa, ARTIFICIAL
  SYNTHETICALLY PRODUCED
LRPXGXSXX

SEQ ID NO: 22 - protein, 10 aa, ARTIFICIAL
  SYNTHETICALLY PRODUCED
XLRPXGXSXX

SEQ ID NO: 23 - protein, 10 aa, ARTIFICIAL
  SYNTHETICALLY PRODUCED
LXRPXGXSXX

SEQ ID NO: 24 - protein, 10 aa, ARTIFICIAL
  SYNTHETICALLY PRODUCED
LXRPXGXSXX

SEQ ID NO: 25 - protein, 10 aa, ARTIFICIAL
  SYNTHETICALLY PRODUCED
LXRPXGXSXX

SEQ ID NO: 26 - protein, 11 aa, ARTIFICIAL
  SYNTHETICALLY PRODUCED
LXXRPXGXSXX

SEQ ID NO: 27 - protein, 13 aa, ARTIFICIAL
  SYNTHETICALLY PRODUCED
CGGGKRPPGFSPL

SEQ ID NO: 28 - protein, 15 aa, ARTIFICIAL
  SYNTHETICALLY PRODUCED
CGGGGGKRPPGFSPL

SEQ ID NO: 29 - protein, 15 aa, ARTIFICIAL
  SYNTHETICALLY PRODUCED
CGGGGGKKRPGFSPL

SEQ ID NO: 30 - protein, 17 aa, ARTIFICIAL
  SYNTHETICALLY PRODUCED
CGGGGGKRKRPPGFSPL

SEQ ID NO: 31 - protein, 12 aa, ARTIFICIAL
  SYNTHETICALLY PRODUCED
CGXKRPPGFSPL

SEQ ID NO: 32 - protein, 16 aa, ARTIFICIAL
  SYNTHETICALLY PRODUCED
CGGGGGLLRPPGXSXI

SEQ ID NO: 33 - protein, 16 aa, ARTIFICIAL
  SYNTHETICALLY PRODUCED
CGGGGGKKRPXGXSXX

SEQ ID NO: 34 - protein, 18 aa, ARTIFICIAL
  SYNTHETICALLY PRODUCED
CGGGGGGGKKRPXGXSXX

SEQ ID NO: 35 - protein, 16 aa, ARTIFICIAL
  SYNTHETICALLY PRODUCED
CGGGGGKKRPXGXSXX

SEQ ID NO: 36 - protein, 10 aa, ARTIFICIAL
  SYNTHETICALLY PRODUCED
KKRPXGXSXX

SEQ ID NO: 37 - protein, 10 aa, ARTIFICIAL
  SYNTHETICALLY PRODUCED
KKRPXGXSXX

SEQ ID NO: 38 - protein, 10 aa, ARTIFICIAL
  SYNTHETICALLY PRODUCED
CKRPPGFSPL

SEQ ID NO: 39 - protein, 16 aa, ARTIFICIAL
  SYNTHETICALLY PRODUCED
CGGGGGXLRPXGXSXX

SEQ ID NO: 40 - protein, 16 aa, ARTIFICIAL
  SYNTHETICALLY PRODUCED
CGGGGGXLRPXGXSXX

SEQ ID NO: 41 - protein, 16 aa, ARTIFICIAL
  SYNTHETICALLY PRODUCED
CGGGGGLXRPXGXSXX

SEQ ID NO: 42 - protein, 15 aa, ARTIFICIAL
  SYNTHETICALLY PRODUCED
GGGGGKKRPPGFSPL

SEQ ID NO: 43 - protein, 10 aa, ARTIFICIAL
  SYNTHETICALLY PRODUCED
XKRPXGXSXX

SEQ ID NO: 44 - protein, 10 aa, ARTIFICIAL
  SYNTHETICALLY PRODUCED
XLRPXGXSXX

SEQ ID NO: 45 - protein, 10 aa, ARTIFICIAL
  SYNTHETICALLY PRODUCED
XLRPXGXSXX

SEQ ID NO: 46 - protein, 10 aa, ARTIFICIAL
  SYNTHETICALLY PRODUCED
XLRPXGXSXX

SEQ ID NO: 47 - protein, 10 aa, ARTIFICIAL
  SYNTHETICALLY PRODUCED
XLRPXGXSXX

SEQ ID NO: 48 - protein, 10 aa, ARTIFICIAL
  SYNTHETICALLY PRODUCED
XLRPXGXSXX

SEQ ID NO: 49 - protein, 10 aa, ARTIFICIAL
  SYNTHETICALLY PRODUCED
XXRPXGXSXX

SEQ ID NO: 50 - protein, 10 aa, ARTIFICIAL
  SYNTHETICALLY PRODUCED
LXRPXGXSXX

SEQ ID NO: 51 - protein, 9 aa, ARTIFICIAL
  SYNTHETICALLY PRODUCED
XRXPGXSXI

SEQ ID NO: 52 - protein, 9 aa, ARTIFICIAL
  SYNTHETICALLY PRODUCED
XRXPGXSXI

SEQ ID NO: 53 - protein, 10 aa, ARTIFICIAL
  SYNTHETICALLY PRODUCED
XLRXPGXSXI

SEQ ID NO: 54 - protein, 10 aa, ARTIFICIAL
  SYNTHETICALLY PRODUCED
XLRXPGXSXI

SEQ ID NO: 55 - protein, 9 aa, ARTIFICIAL
  SYNTHETICALLY PRODUCED
LRPPGFSXI

SEQ ID NO: 56 - protein, 9 aa, ARTIFICIAL
  SYNTHETICALLY PRODUCED
LRPPGFSXI

SEQ ID NO: 57 - protein, 9 aa, ARTIFICIAL
  SYNTHETICALLY PRODUCED
XRXPGXSXI

SEQ ID NO: 58 - protein, 9 aa, ARTIFICIAL
  SYNTHETICALLY PRODUCED
XRXPGXSXI

SEQ ID NO: 59 - protein, 9 aa, ARTIFICIAL
  SYNTHETICALLY PRODUCED
LRXPGXSXI

SEQ ID NO: 60 - protein, 9 aa, ARTIFICIAL
  SYNTHETICALLY PRODUCED
LRXPGXSXI

SEQ ID NO: 61 - protein, 10 aa, ARTIFICIAL
  SYNTHETICALLY PRODUCED
XLRPXGXSXX

SEQ ID NO: 62 - protein, 10 aa, ARTIFICIAL
  SYNTHETICALLY PRODUCED
XLRPXGXSXX

(19) United States
(12) Patent Application Publication
(10) Pub. No.: US 2006/0199812 A1
(43) Pub. Date: Sep. 7, 2006

(54) Method of conjugating aminothiol containing molecules to vehicles

(75) Inventors: Derin C. D'Amico, Newbury Park, CA (US); Benny C. Askew JR., Marshfield, MA (US)

Correspondence Address: AMGEN INC., MAIL STOP 28-2-C, ONE AMGEN CENTER DRIVE, THOUSAND OAKS, CA, 91320-1799, US

(73) Assignee: Amgen Inc.

(21) Appl. No.: 11/338,371

(22) Filed: Jan. 23, 2006

Related U.S. Application Data
(60) Provisional application No. 60/646,685, filed on Jan. 24, 2005.

(51) Int. Cl. A61K 31/542; C07D 498/14; C07D 498/04

(52) U.S. Cl. 514/224.2; 514/224.5; 544/14; 544/48

(57) ABSTRACT

The present invention relates to a novel chemical process that provides novel vehicle derivatives that are exceptional 1,2- or 1,3-aminothiol specific reagents for conjugation to unprotected targeted compounds (e.g., polypeptides, peptides, or organic compounds) having or modified to have a 1,2- or 1,3 aminothiol group. The invention further relates to the methods of using novel water-soluble polymer derivatives and conjugates thereof.

This application claims the benefit of U.S. Provisional Application No. 60/646,685, filed Jan. 24, 2005, which is hereby incorporated by reference.

### BACKGROUND OF THE INVENTION

Recent advances in biotechnology allow large scale manufacturing of biomolecules such as therapeutic proteins, peptides, antibodies, and antibody fragments, making such biomolecules more widely available. Unfortunately, the usefulness of biomolecules is often hampered by their rapid proteolytic degradation, short circulating half-life, low solubility, instability upon manufacture, storage or administration, or by their immunogenicity upon administration. Due to the growing interest in administering biomolecules for therapeutic and/or diagnostic use, various approaches to overcome these deficiencies have been explored.

One such approach that has been widely explored is the modification of proteins and other potentially therapeutic agents by covalent attachment of a vehicle such as polyethylene glycol (hereinafter, “PEG”) (for example, see Abuchowski, A., et al., J. Biol. Chem. 252(11): 3579-3586 (1977); Davis, S., et al., Clin. Exp. Immunol., 46:649-652 (1981); and U.S. Patent Application Publication No. 20040132664). The process of attaching a PEG group (hereinafter, “pegylation”) to a protein or peptide, to solve or ameliorate many of the problems of protein or peptide pharmaceuticals is well documented (see, for example, Francis, et al., International Journal of Hematology, 68:1-18 (1998); Abuchowski, A., et al., (1977); Chapman, A., Adv. Drug Del. Rev. 54, 531-545 (2002)); and Roberts, M. J., et al., Advanced Drug Delivery Reviews, 54:459-476 (2002)).

Briefly stated, covalent attachment of a vehicle to an active agent such as a protein, peptide, polysaccharide, polynucleotide, lipid, or an organic molecule (hereinafter, “conjugation”) is typically accomplished using a vehicle derivative having a reactive group at one or both termini. The reactive group is chosen based on the type of reactive group available on the molecule that will be coupled to the vehicle. By way of example, means to functionalize polymers are provided in WO96/41813 and J. Pharmaceut. Sci. 87, 1446-1449 (1998)). When the vehicle is PEG, activated PEG derivatives suitable for reaction with a nucleophilic center of a biomolecule (e.g., lysine, cysteine and similar residues of proteins or peptides) include PEG-aldehydes, mixed anhydrides, N-hydroxysuccinimide esters, carbonylimadazolides, and chlorocyanurates. Each of these methodologies have known advantages and disadvantages (Harris, J. M., Herati, R. S., Polym Prepr. (Am. Chem. Soc., Div. Polyin. Chem), 32(1):154-155 (1991); Herman, S., et al., Macromol. Chem. Phys., 195:203-209 (1994); and Roberts, M. J., et al., Advanced Drug Delivery Reviews, 54:459-476 (2002)). Some of the more common problems associated with conjugation using known methodologies include the generation of reactive impurities, unstable linkages, side reactions, and/or lack of selectivity in substitution. Furthermore, these difficulties manifest themselves by complicating the isolation and purification of the desired bioactive conjugate. In some cases, isomers are produced in varying amounts. Such variability has the potential of introducing lot-to-lot reproducibility problems, the most problematic of which may result in irreproducible bioactivity.

Activated vehicle derivatives having a thiol-selective functional group such as maleimides, vinyl sulfones, iodoacetamides, thiols, and disulfides are particularly suited for coupling to the cysteine side chains of proteins or peptides (Zalipsky, S. Bioconjug. Chem. 6, 150-165 (1995); Greenwald, R. B. et al. Crit. Rev. Ther. Drug Carrier Syst. 17, 101-161 (2000); 25 Herman, S., et al., Macromol. Chem. Phys. 195, 203-209 (1994)). However, these reagents are also not without their shortcomings especially if the goal is to develop a vehicle-conjugated biomolecule for therapeutic use. For example, the PEG maleimide-thiol conjugate formed initially is a mixture of (R)— and (S)-chirality. Formation of mixtures complicates development of the PEGylated biomolecule on many levels. For example, one of the enantiomers may have undesirable activities or untoward safety issues as compared to the other. Another shortcoming of PEG maleimide-thiol conjugation methodology is that the adduct formed initially is prone to rearrangement to a thiomorpholinone.

The need to reproducibly create conjugates of two or more linked active agents also exists. In certain cases, the administration of these “multimeric” complexes that contain more than one active agent attached to the same molecule of a vehicle leads to additional and/or synergistic benefits. For example, a complex containing two or more identical binding peptides or polypeptides may have substantially increased affinity for the ligand or active site to which it binds relative to the monomeric polypeptide. Alternatively, a complex comprised of (1) a bioactive protein that exerts its effect at a particular site in the body and (2) a molecule that can direct the complex to that specific site may be particularly beneficial. Unfortunately, extending the present methodologies to produce a vehicle conjugated with more than a single bioactive or biofunctional molecule amplifies the deficiencies mentioned above. Attempts to conjugate two bioactive molecules to a single bivalent PEG-maleimide, for example, may result in 16 discrete entities in varying amounts. Applying the current methodologies to the generation of a PEG conjugated with a total of four bioactive molecules through the use of a tetravalent PEG-maleimide, for example, allows for 256 potential discrete attachment sites between PEG and the bioactive molecules, and so on. Trying to quantitate these discrete entities is generally a difficult, and sometimes even an impossible, technical challenge with existing tools and may greatly impede or even altogether thwart the development of biomolecules of this type

Accordingly, there exists a clear need for novel methods of preparing conjugates of active agents in high yields and purity. Ideally, such conjugates are hydrolytically stable, require a relative minimal number of reactions to generate, are readily purified using processes that maintain the integrity of the vehicle or vehicle segments (i.e., is carried out under mild reaction conditions) and/or retain desirable bioactivity. The present invention provides novel reagents, methods, and conjugates that solve the aforementioned problems that presently exist in the state of the art and provides many advantages relative thereto.

### SUMMARY OF THE INVENTION

The present invention relates to vehicle derivatives comprising at least one vehicle segment having a 1,2- or 1,3-aminothiol-selective terminus. The vehicle derivatives of the present invention are useful for coupling to molecules comprising a 1,2- or 1,3-aminothiol moiety. One embodiment of the invention relates to the attachment of one or more active agents to a water-soluble polymer including, but not limited to, PEG.

The present invention provides methods of making the vehicle derivatives of the invention and methods of using the vehicle derivatives to make novel conjugates of active agents.

One aspect of the invention relates to a compound having the structure: 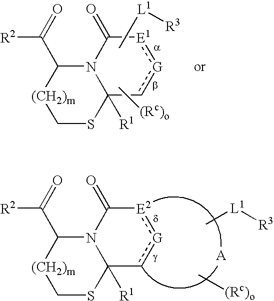
or a pharmaceutically acceptable salt or hydrate thereof, wherein:

A is a saturated, partially-saturated, or unsaturated 2-, 3-, 4-, 5- or 6-atom bridge containing 0, 1, 2, or 3 heteroatoms selected from O, N, and S, with the remaining bridge atoms being carbon;

E¹ is N, O, or C;

E² is N or C;

G is a single bond, a double bond, C, N, O, B, S, Si, P, Se, or Te; 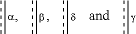
are each a single bond and one of 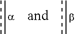
may additionally be a double bond; and when G is C or N one of 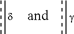
may additionally be a double bond; and when G is a single bond or a double bond, 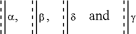
are all absent;

L¹ is a divalent C₁₋₆alkyl or C₁₋₆heteroalkyl, both of which are substituted by 0, 1, 2, or 3 substituents selected from F, Cl, Br, I, OR^{a}, NR^{a}R^{a} and oxo;

m is independently in each instance, 0 or 1;

o is 0, 1, 2, 3 , 4 or 5;

R¹ is H, C₁₋₆-alkyl, phenyl or benzyl, any of which is substituted by 0, 1, 2, or 3 groups selected from halo, cyano, nitro, oxo, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from F, Br, Cl and I;

R² is a vehicle and R³ a bioactive compound; or R³ is a vehicle and R² a bioactive compound;

R^{a} is independently, at each instance, H or R^{b};

R^{b} is independently, at each instance, phenyl, benzyl or C₁₋₆alkyl, the phenyl, benzyl and C₁₋₆alkyl being substituted by 0, 1, 2, or 3 substituents selected from halo, C₁₋₄alkyl, C₁₋₃haloalkyl, —OC₁₋₄alkyl, OH, —NH₂, —NHC₁₋₄alkyl, and —N(C₁₋₄alkyl)C₁₋₄alkyl; and

R^{c} is independently, in each instance, selected from halo, C₁₋₄alkyl, C₁₋₃haloalkyl, —OC₁₋₄alkyl, OH, —NH₂, —NHC₁₋₄alkyl and —N(C₁₋₄alkyl)C₁₋₄alkyl.

Another aspect of the invention relates to a compound having the structure: 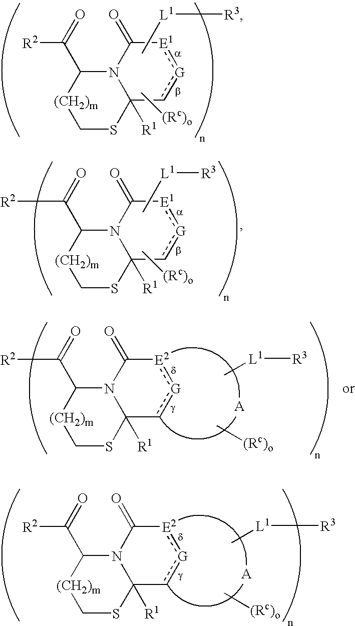
or a pharmaceutically acceptable salt or hydrate thereof, wherein:

A is a saturated, partially-saturated, or unsaturated 2-, 3-, 4-, 5- or 6-atom bridge containing 0, 1, 2, or 3 heteroatoms selected from O, N, and S, with the remaining bridge atoms being carbon;

E¹ is N, O, or C;

E² is N or C;

G is a single bond, a double bond, C, N, O, B, S, Si, P, Se, or Te; 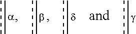
are each a single bond and one of 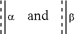
may additionally be a double bond; and when G is C or N one of 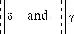
may additionally be a double bond; and when G is a single bond or a double bond, 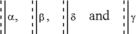
are all absent;

L¹ is a divalent C₁₋₆alkyl or C₁₋₆heteroalkyl, both of which are substituted by 0, 1, 2, or 3 substituents selected from F, Cl, Br, I, OR^{a}, NR^{a}R^{a} and oxo;

m is independently in each instance, 0 or 1;

n is greater than or equal to 1;

o is 0, 1, 2, 3, 4 or 5;

R¹ is H, C₁₋₆alkyl, phenyl or benzyl, any of which is substituted by 0, 1, 2, or 3 groups selected from halo, cyano, nitro, oxo, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}and —NR^{a}C₂₋₆alkylOR^{a}, and additionally substituted by 0, 1, 2, 3, 4, 5 or 6 atoms selected from F, Br, Cl and I;

R² is a vehicle and R³ a bioactive compound; or R³is a vehicle and R² a bioactive compound;

R^{a} is independently, at each instance, H or R^{b};

R^{b} is independently, at each instance, phenyl, benzyl or C₁₋₆alkyl, the phenyl, benzyl and C₁₋₆alkyl being substituted by 0, 1, 2, or 3 substituents selected from halo, C₁₋₄alkyl, C₁₋₃haloalkyl, —OC₁₋₄alkyl, OH, —NH₂, —NHC₁₋₄alkyl, and —N(C₁₋₄alkyl)C₁₋₄alkyl; and

R^{c} is independently, in each instance, selected from halo, C₁₋₄alkyl, C₁₋₃haloalkyl, —OC₁₋₄alkyl, OH, —NH₂, —NHC₁₋₄alkyl and —N(C₁₋₄alkyl)C₁₋₄alkyl.

In another embodiment, in conjunction with the above and below embodiments, A is a saturated, partially-saturated, or unsaturated 2-, 3-, 4-, 5- or 6-atom bridge containing 1, 2, or 3 heteroatoms selected from O, N, and S, with the remaining bridge atoms being carbon.

In another embodiment, in conjunction with the above and below embodiments, A is a saturated, partially-saturated, or unsaturated 2-, 3-, 4-, 5- or 6-carbon-atom bridge.

In another embodiment, in conjunction with the above and below embodiments, n is 1.

In another embodiment, in conjunction with the above and below embodiments, n is 2.

In another embodiment, in conjunction with the above and below embodiments, n is 3.

In another embodiment, in conjunction with the above and below embodiments, n is 4.

In another embodiment, in conjunction with the above and below embodiments, n is 5.

In another embodiment, in conjunction with the above and below embodiments, n is 6.

In another embodiment, in conjunction with the above and below embodiments, n is 7.

In another embodiment, in conjunction with the above and below embodiments, n is 8.

In another embodiment, in conjunction with the above and below embodiments, A is a an unsaturated 4-carbon-atom bridge; E² is C; and G is a double bond.

In another embodiment, in conjunction with the above and below embodiments, G is a single bond or a double bond and 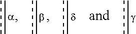
are all absent.

In another embodiment, in conjunction with the above and below embodiments, G is C, N, O, B, S, Si, P, Se, or Te.

In another embodiment, in conjunction with the above and below embodiments, 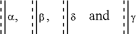
are each a single bond.

In another embodiment, in conjunction with the above and below embodiments, G is C or N; and one of 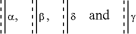
is a double bond.

In another embodiment, in conjunction with the above and below embodiments, R² is a vehicle and R³ a bioactive compound.

In another embodiment, in conjunction with the above and below embodiments, R³ is a vehicle and R² a bioactive compound.

In another embodiment, in conjunction with the above and below embodiments, R³ selected from poly(alkylene oxide), poly(vinyl pyrrolidone), poly(vinyl alcohol), polyoxazoline, poly(acryloylmorpholine-), poly(oxyethylated polyol), poly(ethylene glycol), carboxymethylcellulose, dextran, polyvinyl alcohol, polyvinyl pyrrolidone, poly-1,3-dioxolane, poly-1,3,6-trioxane, an amino acid homopolymer, polypropylene oxide, a copolymer of ethylene glycol/propylene glycol, an ethylene/maleic anhydride copolymer, an amino acid copolymer, a copolymer of PEG and an amino acid, a polypropylene oxide/ethylene oxide copolymer, and a polyethylene glyco/thiomalic acid copolymer; or any combination thereof.

In another embodiment, in conjunction with the above and below embodiments, R³ is PEG.

In another embodiment, in conjunction with the above and below embodiments, n is 1, 2, 3, 4, 5, 6, 7, 8, 9 or 10.

In another embodiment, in conjunction with the above and below embodiments, R³ is a branched PEG and n is 2, 3, 4, 5, 6, 7, 8, 9 or 10.

In another embodiment, in conjunction with the above and below embodiments, R² is a B1 peptide antagonist.

In another embodiment, in conjunction with the above and below embodiments, R² is a B1 peptide antagonist selected from SEQ ID NOS:5-26 and 42-62 wherein said peptide was modified to have a N-terminal cysteine residue.

Another aspect of the invention relates to a method for preparing a compound according to Claim 1, comprising the step of reacting:
R²—(C(═O))ₘCH(NH₂)CH₂(CH₂)ₘSH with A) 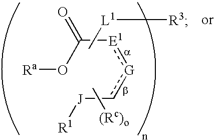 R²—[(C(═O))ₘCH(NH₂)CH₂(CH₂)ₘSH]ₙ with B) 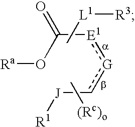
wherein J is a carbonyl or a protected version thereof.

Another aspect of the invention relates to a method for preparing a compound according to claim 1, comprising the step of reacting:
R²—(C(═O))ₘCH(NH₂)CH₂(CH₂)ₘSH with A) 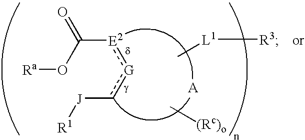 R²—[(C(═O))ₘCH(NH₂)CH₂(CH₂)ₘSH]ₙ with B)

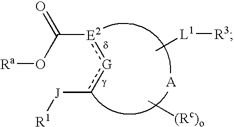

wherein J is a carbonyl or a protected version thereof.

In another embodiment, in conjunction with the above and below embodiments, J is selected from C(═O), C(OCH₂CH₂O), C(N(R^{a})CH₂CH₂N(R^{a})), C(N(R^{a})CH₂CH₂O), C(N(R^{a})CH₂CH₂S), C(OCH₂CH₂CH₂O), C(N(R^{a})CH₂CH₂CH₂N(R^{a})), C(N(R^{a})CH₂CH₂CH₂O), C(N(R^{a})CH₂CH₂CH₂S), C(OR^{b})₂, C(SR^{b})₂ and C(NR^{a}R^{b})₂.

In another embodiment, in conjunction with the above and below embodiments, the reaction is perfomed at a pH between 2 and 7.

In another embodiment, in conjunction with the above and below embodiments, the reaction is perfomed at a pH between 3 and 5.

Another aspect of the invention relates to a compound having the structure: 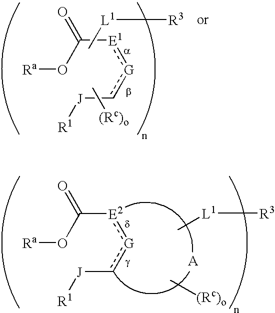
wherein:

A is a saturated, partially-saturated, or unsaturated 2-, 3-, 4-, 5- or 6-atom bridge containing 0, 1, 2, or 3 heteroatoms selected from O, N, and S, with the remaining bridge atoms being carbon;

E¹ is N, O, or C;

E² is N or C;

G is a single bond, a double bond, C, N, O, B, S, Si, P, Se, or Te; 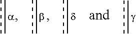
are each a single bond and one of 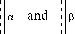
may additionally be a double bond; and when G is C or N one of 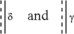
may additionally be a double bond; and when G is a single bond or a double bond, 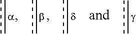
are all absent;

J is a carbonyl or a protected version thereof;

L¹ is a divalent C₁₋₁₂alkyl or C₁₋₁₂heteroalkyl, both of which are substituted by 0, 1, 2, or 3 substituents selected from F, Cl, Br, I, OR^{a}, NR^{a}R^{a} and oxo;

m is independently in each instance, 0 or 1;

n is 1, 2, 3, 4, 5, 6, 7, 8, 9 or 10;

o is 0, 1, 2, 3, 4 or 5;

R¹ is H, C₁₋₆alkyl, phenyl or benzyl, any of which is substituted by 0, 1, 2, or 3 groups selected from halo, cyano, nitro, oxo, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from F, Br, Cl and I;

R³ is a bioactive compound or a vehicle;

R^{a} is independently, at each instance, H or R^{b};

R^{b} is independently, at each instance, phenyl, benzyl or C₁₋₆alkyl, the phenyl, benzyl and C₁₋₆alkyl being substituted by 0, 1, 2, or 3 substituents selected from halo, C₁₋₄alkyl, C₁₋₃haloalkyl, —OC₁₋₄alkyl, OH, —NH₂, —NHC₁₋₄alkyl, and —N(C₁₋₄alkyl)C₁₋₄alkyl;

R^{c} is independently, in each instance, selected from halo, C₁₋₄alkyl, C₁₋₃haloalkyl, —OC₁₋₄alkyl, OH, —NH₂, —NHC₁₋₄alkyl and —N(C₁₋₄alkyl)C₁₋₄alkyl; and

X is C(═O) and Y is NH; or X is NH and Y is C(═O).

In another embodiment, in conjunction with the above and below embodiments, n is 1.

In another embodiment, in conjunction with the above and below embodiments, n is 2.

In another embodiment, in conjunction with the above and below embodiments, n is 3.

In another embodiment, in conjunction with the above and below embodiments, n is 4.

In another embodiment, in conjunction with the above and below embodiments, n is 5.

In another embodiment, in conjunction with the above and below embodiments, n is 6.

In another embodiment, in conjunction with the above and below embodiments, n is 7.

In another embodiment, in conjunction with the above and below embodiments, n is 8.

In another embodiment, in conjunction with the above and below embodiments, A is a saturated, partially-saturated, or unsaturated 2-, 3-, 4-, 5- or 6-atom bridge containing 1, 2, or 3 heteroatoms selected from O, N, and S, with the remaining bridge atoms being carbon.

In another embodiment, in conjunction with the above and below embodiments, A is a saturated, partially-saturated, or unsaturated 2-, 3-, 4-, 5- or 6-carbon-atom bridge.

In another embodiment, in conjunction with the above and below embodiments, A is an unsaturated 4-carbon-atom bridge; E² is C; and G is a double bond.

In another embodiment, in conjunction with the above and below embodiments, G is a single bond or a double bond and 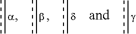
are all absent.

In another embodiment, in conjunction with the above and below embodiments, G is C, N, O, B, S, Si, P, Se, or Te.

In another embodiment, in conjunction with the above and below embodiments, 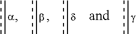
are each a single bond.

In another embodiment, in conjunction with the above and below embodiments, G is C or N; and one of 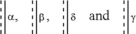
is a double bond.

In another embodiment, in conjunction with the above and below embodiments, R³ a bioactive compound.

In another embodiment, in conjunction with the above and below embodiments, R³ is a vehicle.

In another embodiment, in conjunction with the above and below embodiments, R³ selected from poly(alkylene oxide), poly(vinyl pyrrolidone), poly(vinyl alcohol), polyoxazoline, poly(acryloylmorpholine-), poly(oxyethylated polyol), poly(ethylene glycol), carboxymethylcellulose, dextran, polyvinyl alcohol, polyvinyl pyrrolidone, poly-1,3-dioxolane, poly-1,3,6-trioxane, an amino acid homopolymer, polypropylene oxide, a copolymer of ethylene glycol/propylene glycol, an ethylene/maleic anhydride copolymer, an amino acid copolymer, a copolymer of PEG and an amino acid, a polypropylene oxide/ethylene oxide copolymer, and a polyethylene glyco/thiomalic acid copolymer; or any combination thereof.

In another embodiment, in conjunction with the above and below embodiments, R³ is PEG.

Another aspect of the invention relates to a method for preparing a compound as described above, comprising the step of reacting (Y-L²)ₙR³ with

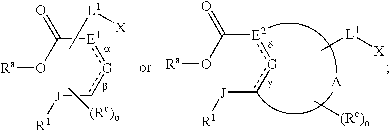

L² is independently, in each instance C₁₋₆alkyl or C₁₋₆heteroalkyl both of which are substituted by 0, 1, 2, 3 or 4 substituents selected from F, Cl, Br, I, OR^{a}, NR^{a}R^{a} and oxo;

X is a nucleophile and Y is an electrophile; or X is an electrophile and Y is a nucleophile.

In another embodiment of the invention, the nucleophile is selected from NH₂ and OH; and the electrophile is selected from CH₂halogen, CH2 SO₂OR^{b}, C(═O)NR^{a}R^{b} and C(═O)OR^{b}.

Another aspect of the invention relates to method of treating pain and/or inflammation comprising the administration to a patient in need thereof of a therapeutically-effective amount of a compound as described above.

Another aspect of the invention relates to a pharmaceutical composition comprising a compound as described above and a pharmaceutically acceptable carrier or dilluent.

Another aspect of the invention relates to the manufacture of a medicament comprising a compound as described above.

Another aspect of the invention relates to the manufacture of a medicament for the treatment of pain and/or inflammation comprising a compound as described above.

One aspect of the invention relates to a compound having the structure: 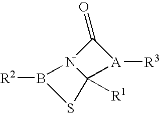
or any pharmaceutically acceptable salts or hydrates thereof, wherein:

A is selected from i) 2-carbons, either sp³- or sp² hybridized (substituted or unsubstituted), wherein both carbons are either cyclic or acyclic, connecting both carboxyls of the electrophile, or ii) 3-atoms selected from carbon (substituted or unsubstituted, part of a ring or acyclic), nitrogen (substituted or unsubstituted, part of a ring or acyclic) or oxygen (part of a ring or acyclic); and

B is selected from i) 2-carbons, either sp³- or sp² hybridized (substituted or unsubstituted), wherein both carbons are either cyclic or acyclic, connecting both carboxyls of the electrophile, or ii) 3-atoms selected from carbon (substituted or unsubstituted, part of a ring or acyclic), nitrogen (substituted or unsubstituted, part of a ring or acyclic) or oxygen (part of a ring or acyclic).

In one embodiment, in conjunction with the above and below embodiments, R¹ is H, C₁₋₆alkyl, phenyl or benzyl, any of which is substituted by 0, 1, 2, or 3 groups selected from halo, cyano, nitro, oxo, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from F, Br, Cl and I;

In one embodiment, in conjunction with the above and below embodiments, R³ selected from poly(alkylene oxide), poly(vinyl pyrrolidone), poly(vinyl alcohol), polyoxazoline, poly(acryloylmorpholine-), poly(oxyethylated polyol), poly(ethylene glycol), carboxymethylcellulose, dextran, polyvinyl alcohol, polyvinyl pyrrolidone, poly-1,3-dioxolane, poly-1,3,6-trioxane, an amino acid homopolymer, polypropylene oxide, a copolymer of ethylene glycol/propylene glycol, an ethylene/maleic anhydride copolymer, an amino acid copolymer, a copolymer of PEG and an amino acid, a polypropylene oxide/ethylene oxide copolymer, and a polyethylene glyco/thiomalic acid copolymer; or any combination thereof.

In another embodiment, in conjunction with the above and below embodiments, said vehicle segment is a poly(ethylene oxide).

In another embodiment, in conjunction with the above and below embodiments, said vehicle is a linear structure.

In another embodiment, in conjunction with the above and below embodiments, said vehicle is a PEG.

In another embodiment, in conjunction with the above and below embodiments, said polycyclic N, S-heterocycle is a (9bS)(9bH)-2,3-dihydrothiazolo[2,3-a]isoindol-5-one, R² is a protein or peptide, and R³ is PEG.

In another embodiment, in conjunction with the above and below embodiments, R² is a B1 peptide antagonist.

In another embodiment, in conjunction with the above and below embodiments, B1 peptide antagonist is a peptide selected from SEQ ID NOS:5-26 and 42-62 wherein said peptide was modified to have a N-terminal cysteine residue.

In another embodiment, in conjunction with the above and below embodiments, said vehicle is a forked or branched structure having two or more water-soluble segments, respectively.

In another embodiment, in conjunction with the above and below embodiments, said vehicle is a branched PEG (bPEG) or a forked PEG (fPEG) having two or more PEG segments.

In another embodiment, in conjunction with the above and below embodiments, said polycyclic N, S-heterocycle is a (9bS)(9bH)-2,3-dihydrothiazolo[2,3-a]isoindol-5-one, R² is a protein or peptide.

In another embodiment, in conjunction with the above and below embodiments, said bPEG has from 3 to 8 polymer segments -(bPEG)₃₋₈.

In another embodiment, in conjunction with the above and below embodiments, at least one of said segments of said bPEG has a terminus activated with an amine (C-[(bPEG)₃₋₈]-(NH₂)₁₋₈).

In another embodiment, in conjunction with the above and below embodiments, said bPEG has four polymer segments (C-[(bPEG)₄]-(NH₂)₁₋₄) and wherein at least one of said segments have termini activated with an amine.

In another embodiment, in conjunction with the above and below embodiments, at least 50% of said segments have termini activated with an amine.

In another embodiment, in conjunction with the above and below embodiments, at least one of said polymer segments is capped.

In another embodiment, in conjunction with the above and below embodiments, said PEG has a nominal average molecular mass from about 200 to about 100,000 daltons.

In another embodiment, in conjunction with the above and below embodiments, said PEG has a nominal average molecular mass from about 5,000 to about 60,000 daltons.

In another embodiment, in conjunction with the above and below embodiments, said PEG has a nominal average molecular mass from about 10,000 to about 40,000 daltons.

In another embodiment, in conjunction with the above and below embodiments, R² is a B1 peptide antagonist in every instance.

In another embodiment, in conjunction with the above and below embodiments, said B1 peptide antagonist is selected from SEQ ID NOS:27-35 and 38-62.

In another embodiment, in conjunction with the above and below embodiments, R² is a B1 peptide antagonist in one instance.

In another embodiment, in conjunction with the above and below embodiments, R² is a B1 peptide antagonist in two of the four instances.

In another embodiment, in conjunction with the above and below embodiments, R² is a B1 peptide antagonist in three of the four instances.

In another embodiment, in conjunction with the above and below embodiments, each said B1 peptide antagonist is independently selected from SEQ ID NOS: 27-34 and 38-62.

In another embodiment, in conjunction with the above and below embodiments, R² is an active agent other than a B1 peptide antagonist in at least one instance.

Another aspect of the invention relates to a pharmaceutical composition comprising any of the above compounds and a pharmaceutical excipient.

Another aspect of the invention relates to the delivery of a pharmaceutical composition comprising any of the above compounds and a pharmaceutical excipient said administering is parenterally, transmucosally or transdermally.

In another embodiment, in conjunction with the above and below embodiments, said transmucosally is orally, nasally, pulmonarily, vaginally or rectally.

In another embodiment, in conjunction with the above and below embodiments, said parenterally is intra-arterial, intravenous, intramuscular, intradermal, subcutaneous, intraperitoneal, intraventricular, intraocular, intraorbital, or intracranial.

In another embodiment, in conjunction with the above and below embodiments, said administering is orally.

In another embodiment, in conjunction with the above and below embodiments, said polypeptide or peptide comprises a Tat-inhibitory polypeptide, comprising an amino acid sequence of R-Arg-Lys-Lys-Arg-Arg-Gln-Arg-Arg-Arg-X-(biotin)-Cys-NH₂ (SEQ ID NO:63), and biologically and pharmaceutically acceptable salts thereof, stereo, optical and geometrical isomers thereof, including retro inverso analogues, where such isomers exist, as well as the pharmaceutically acceptable salts and solvates thereof, wherein R comprises the residue of a carboxylic acid or an acetyl group; and X is a Cys residue.

In another embodiment, in conjunction with the above and below embodiments, said polypeptide or peptide comprising a aminothiol compound comprises an amino acid sequence selected from N-acetyl-Arg-Lys-Lys-Arg-Arg-Gln-Arg-Arg-Arg-Cys-(biotin)-Cys-NH₂ (SEQ ID NO:64), N-acetyl-Arg-Lys-Lys-Arg-Arg-Gln-Arg-Arg-Arg-Lys-(biotin)-Cys-NH₂ (SEQ ID NO:65), N-acetyl-Arg-Lys-Lys-Arg-Arg-Gln-Arg-Arg-Arg-D-Cys-(biotin)-Cys-NH₂ (SEQ ID NO:66), N-acetyl-Arg-Lys-Lys-Arg-Arg-Gln-Arg-Arg-Arg-D-Lys-(biotin)-Cys-NH₂ (SEQ ID NO:67), N-acetyl-Gln-Lys-Lys-Arg-Arg-Gln-Arg-Arg-Arg-D-Lys-(biotin)-Cys-NH₂ (SEQ ID NO:68), N-acetyl-Arg-Lys-Lys-Arg-Arg-Pro-Arg-Arg-Arg-Cys-(biotin)-Cys-NH₂ (SEQ ID NO:69), N-acetyl-DCys-DLys-(biotin)-DArg-DArg-DArg-DGln-DArg-DArg-DLys-DLys-DArg-NH₂ or biologically and pharmaceutically acceptable salts thereof.

In another embodiment, in conjunction with the above and below embodiments, said vehicle is selected from the group consisting of poly(ethylene glycol), carboxymethylcellulose, dextran, polyvinyl alcohol, polyvinyl pyrrolidone, poly-1,3-dioxolane, poly-1,3,6-trioxane, an amino acid homopolymer, polypropylene oxide, a copolymer of ethylene glycol/propylene glycol, an ethylene/maleic anhydride copolymer, an amino acid copolymer, a copolymer of PEG and an amino acid, a polypropylene oxide/ethylene oxide copolymer, and a PEG/thiomalic acid copolymer, or any combination thereof.

In another embodiment, in conjunction with the above and below embodiments, said polymer has a molecular weight of about 100 to about 200,000 daltons.

In another embodiment, in conjunction with the above and below embodiments, said polymer has a molecular weight of about 2,000 to about 50,000 daltons.

In another embodiment, in conjunction with the above and below embodiments, said interval is about 100 to about 10,000 Daltons.

In another embodiment, in conjunction with the above and below embodiments, said interval is about 300 to about 5,000 Daltons.

Another aspect of the invention relates to a method for preparing a 1,2- or 1,3-aminothiol-selective vehicle derivative comprising the steps of:

(a) providing a vehicle comprising at least one vehicle segment having the formula:
Y—R³
wherein Y is either a nucleophile or an electrophile and R₃ is a vehicle.

(b) reacting said vehicle derivative to form a covalent attachment with a molecule comprising a 1,2- or 1,3-aminothiol selective moiety, or a protected form thereof, having the formula: 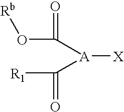
wherein A is i) 2-carbons, either sp³- or sp² hybridized (substituted or unsubstituted), and wherein both carbons are either cyclic or acyclic, connecting both carboxyls of the electrophile, or ii) 3-atoms selected from carbon (substituted or unsubstituted, part of a ring or acyclic), nitrogen (substituted or unsubstituted, part of a ring or acyclic) or oxygen (part of a ring or acyclic); wherein R¹ is selected from H and an electron withdrawing group; wherein R²=alkyl; wherein X is an electrophile when Y is a nucleophile or X is a nucleophile when Y is an electrophile.

In another embodiment, in conjunction with the above and below embodiments, A is a structure having the formula:

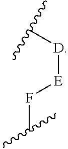

In another embodiment, in conjunction with the above and below embodiments, A is acyclic.

In another embodiment, in conjunction with the above and below embodiments, F is carbon and D is selected from i) carbon ii) oxygen and iii) nitrogen.

In another embodiment, in conjunction with the above and below embodiments, D is carbon, E is selected from carbon substituted by X, nitrogen substituted by X, oxygen, sulfur, silicon substituted by X, boron substituted by X, a bond, phosphorous substituted by X; or ii) oxygen, E is selected from carbon, nitrogen, silicon, boron, and a bond; or iii) nitrogen, E is selected from carbon, nitrogen, oxygen, silicon sulfer, boron, and a bond.

In another embodiment, in conjunction with the above and below embodiments, A is a structure having the formula:

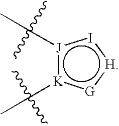

In another embodiment, in conjunction with the above and below embodiments, F is carbon and D is selected from i) carbon ii) oxygen and iii) nitrogen.

In another embodiment, in conjunction with the above and below embodiments, Y is an acid.

In another embodiment, in conjunction with the above and below embodiments, Y is an amine.

In another embodiment, in conjunction with the above and below embodiments, Y is a primary amine.

In another embodiment, in conjunction with the above and below embodiments, greater than 95% of Y is covalently bonded to the 1,2- or 1,3-aminothiol selective moiety.

In another embodiment, in conjunction with the above and below embodiments, at least one of said R₃ is selected from H, alkyl, C₁₋₁₀ linear alkyl, poly(alkylene oxide), poly(vinyl pyrrolidone), poly(vinyl alcohol), polyoxazoline, poly-(acryloylmorpholine-), poly(oxyethylated polyol), and poly(ethylene oxide).

In another embodiment, in conjunction with the above and below embodiments, said vehicle has a branched, forked, or multi-armed structure.

In another embodiment, in conjunction with the above and below embodiments, at least R³is PEG.

In another embodiment, in conjunction with the above and below embodiments, said vehicle has a nominal average molecular mass from about 200 to about 100,000 daltons.

In another embodiment, in conjunction with the above and below embodiments, the method further comprises a first step of purifying said vehicle such that >95% of said segments have termini activated with an amine.

In another embodiment, in conjunction with the above and below embodiments, said purifying step comprises a chromatographic or a chemical separation.

In another embodiment, in conjunction with the above and below embodiments, said purifying step comprises cation exchange chromatography.

In another embodiment, in conjunction with the above and below embodiments, said nucleophile is selected from a secondary amine, hydroxy, imino, or thiol.

In another embodiment, in conjunction with the above and below embodiments, said electrophile is an activated ester.

In another embodiment, in conjunction with the above and below embodiments, said activated ester is selected from a N-hydroxysuccinimidyl, succinimidyl, N-hydroxybenzotriazoyl, perfluorophenyl, alkylating moieties such as chloro-, bromo-, iodoalkanes, activated alcohols such as methanesulfonyl-, trifluoromethanesulfonyl-, p-toluenesulfonyl-, trichloroacetimidate, and in situ activated alcohols such as triphenylphosphonium ethers.

In another embodiment, in conjunction with the above and below embodiments, Y is selected from an alkoxy, substituted alkoxy, alkenyloxy, substituted alkenyloxy, alkynyloxy, substituted alkynyloxy, aryloxy, and substituted aryloxy.

In another embodiment, in conjunction with the above and below embodiments, said PEG has a nominal average molecular mass from about 5,000 to about 60,000 daltons.

In another embodiment, in conjunction with the above and below embodiments, said PEG has a nominal average molecular mass from about 10,000 to about 40,000 daltons.

Another aspect of the invention relates to a method of preparing a composition of matter comprising the steps of:

(a) providing a vehicle comprising at least one vehicle segment having the formula:
Y—R³
wherein Y is either a nucleophile or an electrophile and R₃ is a vehicle.

(b) reacting said vehicle derivative to form a covalent attachment with a molecule comprising a 1,2- or 1,3-aminothiol selective moiety, or a protected form thereof, having the formula: 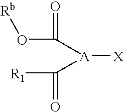
wherein A is i) 2-carbons, either sp³- or sp² hybridized (substituted or unsubstituted), and wherein both carbons are either cyclic or acyclic, connecting both carboxyls of the electrophile, or ii) 3-atoms selected from carbon (substituted or unsubstituted, part of a ring or acyclic), nitrogen (substituted or unsubstituted, part of a ring or acyclic) or oxygen (part of a ring or acyclic); wherein R¹ is selected from H and an electron withdrawing group; wherein X is an electrophile when Y is a nucleophile or X is a nucleophile when Y is an electrophile; and

(c) reacting the predominant product from steps (a) and (b) with an active agent or substrate comprising a 1,2- or 1,3-aminothiol.

In another embodiment, in conjunction with the above and below embodiments, said active agent is a polypeptide or peptide.

In another embodiment, in conjunction with the above and below embodiments, peptide is a B1 peptide antagonist.

In another embodiment, in conjunction with the above and below embodiments, said peptide is a peptide selected from SEQ ID NOS:27-35 and 38-41.

In another embodiment, in conjunction with the above and below embodiments, said peptide is selected from SEQ ID NOS: 11-26 and 43-46 further comprising a cysteine at the N-terminus of said peptide.

In another embodiment, in conjunction with the above and below embodiments, said 1,2- or 1,3-aminothiol-selective moiety is a 1,2- or 1,3-formyl ester.

In another embodiment, in conjunction with the above and below embodiments, said electrophile is an acid.

In another embodiment, in conjunction with the above and below embodiments, said nucleophile is an amine.

In another embodiment, in conjunction with the above and below embodiments, said electrophile is a primary amine.

In another embodiment, in conjunction with the above and below embodiments, said vehicle segment is selected from poly(alkylene oxide), poly(vinyl pyrrolidone), poly(vinyl alcohol), polyoxazoline, poly(acryloylmorpholine-), poly(oxyethylated polyol), poly(ethylene glycol), carboxymethylcellulose, dextran, polyvinyl alcohol, polyvinyl pyrrolidone, poly-1,3-dioxolane, poly-1,3,6-trioxane, an amino acid homopolymer, polypropylene oxide, a copolymer of ethylene glycol/propylene glycol, an ethylene/maleic anhydride copolymer, an amino acid copolymer, a copolymer of PEG and an amino acid, a polypropylene oxide/ethylene oxide copolymer, and a polyethylene glyco/thiomalic acid copolymer; or any combination thereof.

In another embodiment, in conjunction with the above and below embodiments, greater than 95% of said activated termini were covalently bonded to the 1,2- or 1,3-aminothiol selective moiety as determined by 13 C NMR for ¹³C containing activated termini, or other methods currently available for activated termini without a ¹³Carbon.

In another embodiment, in conjunction with the above and below embodiments, said vehicle segment is a poly(ethylene oxide).

In another embodiment, in conjunction with the above and below embodiments, said vehicle segment is a polyethylene glycol (PEG).

In another embodiment, in conjunction with the above and below embodiments, said PEG has a linear, branched (bPEG), forked (fPEG), or multi-armed structure.

In another embodiment, in conjunction with the above and below embodiments, said branched PEG has from 3 to 8 polymer segments (C-[bPEG₃₋₈]).

In another embodiment, in conjunction with the above and below embodiments, at least one of said segments has a terminus activated with an amine (C-[bPEG₃₋₈]-(NH₂)₁₋₈).

In another embodiment, in conjunction with the above and below embodiments, said bPEG has four polymer segments (C-[bPEG₄]-(NH₂)₁₋₄) and wherein at least one of said segments has a terminus activated with an amine.

In another embodiment, in conjunction with the above and below embodiments, at least 50% of the termini of said segments are activated with an amine.

In another embodiment, in conjunction with the above and below embodiments, at least one of said polymer segments is capped.

In another embodiment, in conjunction with the above and below embodiments, said PEG has a nominal average molecular mass from about 200 to about 100,000 daltons.

In another embodiment, in conjunction with the above and below embodiments, the method further comprises a first step of purifying said amine activated vehicle such that >95% of said segments have termini activated with an amine.

In another embodiment, in conjunction with the above and below embodiments, said purifying step comprises a chromatographic or a chemical separation.

In another embodiment, in conjunction with the above and below embodiments, said purifying step comprises cation exchange chromatography.

In another embodiment, in conjunction with the above and below embodiments, said nucleophile is selected from a secondary amine, hydroxy, imino, or thiol.

In another embodiment, in conjunction with the above and below embodiments, said electrophile is an activated ester.

In another embodiment, in conjunction with the above and below embodiments, said activated ester is selected from a N-hydroxysuccinimidyl, succinimidyl, N-hydroxybenzotriazoyl, perfluorophenyl, alkylating moieties such as chloro-, bromo-, iodoalkanes, activated alcohols such as methanesulfonyl, trifluoromethanesulfonyl, p-toluenesulfonyl-, trichloroacetimidate, and in situ activated alcohols such as triphenylphosphonium ethers.

In another embodiment, in conjunction with the above and below embodiments, said cap comprises a chemical group selected from an alkoxy, substituted alkoxy, alkenyloxy, substituted alkenyloxy, alkynyloxy, substituted alkynyloxy, aryloxy, and substituted aryloxy.

In another embodiment, in conjunction with the above and below embodiments, said cap further comprises a radioactive, magnetic, colorimetric, or fluorescent group.

In another embodiment, in conjunction with the above and below embodiments, said PEG has a nominal average molecular mass from about 5,000 to about 60,000 daltons.

In another embodiment, in conjunction with the above and below embodiments, said PEG has a nominal average molecular mass from about 10,000 to about 40,000 daltons.

In another embodiment, in conjunction with the above and below embodiments, said polypeptide or peptide is selected from a biological transporter, receptor, binding or targeting ligands that can be any moiety binding to a cell surface component, including but not limited to vitamins (e.g. biotin, folate, pantothenate, B-6, B-12), sugars (e.g. glucose, N-acetyl glucosamine), chemokines (e.g. RANTES, IL-2, OPG), peptide (or non-peptide) vectors (e.g. Tat, fMLF, penetratin, VEGF [a glycoprotein], transferrin), Retro inverso peptides (e.g. RI TAT), membrane fusion peptides (e.g. gp41, VEGF [a glycoprotein]), lipids (or phospholipids) (e.g. myristic acid, stearic acid), sense (or antisense) oligonucleotides (e.g. aptamers containing 5-(1-pentyl)-2′-deoxyuridine), enzymes (e.g. neuraminidase), toxins, antibodies (or antibody fragments) (e.g. CD4 [targets helper T cells], CD44 [targets ovarian cancer cells]), antigens (or epitopes) (e.g. influenza virus hemagglutinin), peptide ligands, hormones (e.g. estrogen, progesterone, LHRH, ACTH, growth hormone), adhesion molecules (e.g. lectins, ICAM) and analogues of any of the foregoing.

In another embodiment, in conjunction with the above and below embodiments, said active agent comprises a 1,2- or 1,3 aminothiol group or is derivatized to have a 1,2- or 1,3 aminothiol group.

Another aspect of the invention relates to a method for identifying a suitable compound for therapeutic or diagnostic use without the components thereof negatively affecting the biological activity of the peptide or protein component of the compound, the method comprising preparing a compound of the present invention and screening the compound for biological activity of the therapeutic and/or diagnostic portion of the compound.

A particular embodiment of the present invention is a method for preparing a 1,2- or 1,3-aminothiol-selective derivative of a vehicle, said method comprising the steps of:

(a) providing a vehicle having at least one vehicle segment having at least one terminus activated with a nucleophile or an electophile; and

(b) reacting said polymer to form a covalent attachment with a molecule comprising a 1,2- or 1,3-aminothiol selective moiety, or a protected form thereof, defined by general Formula I: 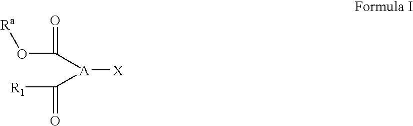
to form a vehicle derivative comprising a 1,2- or 1,3-aminothiol-selective terminus, or a protected form thereof, wherein A is i) 2-carbons, either sp³- or sp² hybridized (substituted or unsubstituted), and wherein both carbons are either cyclic or acyclic, connecting both carboxyls of the electrophile, or ii) 3-atoms selected from carbon (substituted or unsubstituted, part of a ring or acyclic), nitrogen (substituted or unsubstituted, part of a ring or acyclic) or oxygen (part of a ring or acyclic).

Another embodiment of the present invention is method of preparing a composition of matter comprising the steps of:

(a) providing a vehicle having at least one vehicle segment activated with a nucleophile or an electophile;

(b) reacting said vehicle to form a covalent attachment with an agent comprising a 1,2- or 1,3-aminothiol selective moiety, or a protected form thereof, defined by general Formula I, wherein A is i) 2-carbons, either sp³- or sp² hybridized (substituted or unsubstituted), and wherein both carbons are either cyclic or acyclic, connecting both carboxyls of the electrophile, or ii) 3-atoms selected from carbon (substituted or unsubstituted, part of a ring or acyclic), nitrogen (substituted or unsubstituted, part of a ring or acyclic) or oxygen (part of a ring or acyclic); and

(c) reacting the predominant product of step (a) and (b) with a active agent comprising a 1,2- or 1,3-aminothiol. Such a method can be depicted generically by Reaction Scheme 1 shown below:

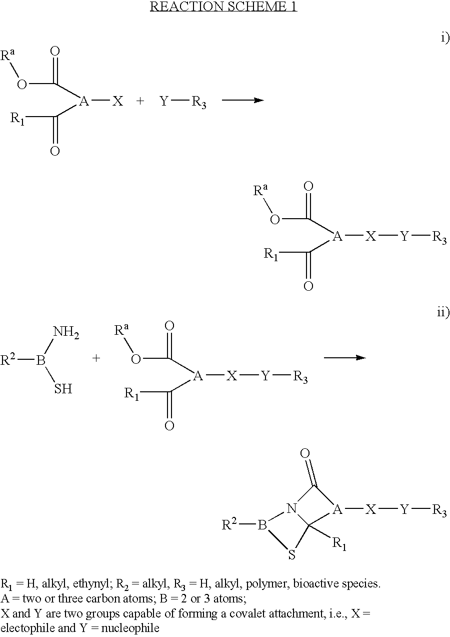

The reaction generically illustrated above (REACTION SCHEME 1) is particularly advantageous when the vehicle is a multivalent vehicle comprising multiple activated vehicle segments making up a multivalent vehicle. In such cases, the methods of the present invention efficiently produce high yields and relatively pure conjugates functionalized at practically each appropriately activated vehicle segment (as defined herein) of the polymer.

In one embodiment, multiple agents may be conjugated to a single branched vehicle. In a non-limiting example, the invention provides biocompatible, water-soluble polymers with multiple branches conjugated to peptide antagonists.

According to features and principles consistent with the invention, various agents may be efficiently conjugated to an activated vehicle via an appropriate reactive group of the agent. Such agents include, but are not limited to, biologically active or diagnostic agents.

In another embodiment of the invention, in conjunction with the above and below embodiments, the agent may be a small-molecule compound with a pharmacological activity. Alternatively, the agent may be a retro-inverso form or optimized form of a biologically-active peptide, possessing the same or similar biological activity of the original form but possessing other desirable characteristics such as decreased susceptibility to enzymatic attack or metabolic enzymes. More particularly, the agent may include, but are not limited to, an antibody or antibody fragment. An agent comprising a suitable 1,2- or 1,3-aminothiol group may be synthetically derived or naturally-occuring within the particular agent. Accordingly, the agent may be an agent having or modified to have 1,2- or 1,3-group, or be conjugatable to a compound having a 1,2- or 1,3-aminothiol group, such as a modified peptide or a cysteine containing bioactive agent.

One exemplary aspect of the present invention includes methods of making vehicle-conjugated B1 peptide antagonists including, but not limited to, the vehicle conjugated B1 peptide antagonists described in pending U.S. application Ser. No. 10/972,236 filed on Oct. 21, 2004 which was published as U.S. Patent Application Publication No. 2005/0215470 on Sep. 29, 2005 (herein after “U.S. Application '236”).

Another object of the present invention is to provide a pharmaceutical composition comprising excipient carrier materials having at least one vehicle-conjugated agent of the invention dispersed therein.

Another object of the present invention is to provide methods of treating a B1 mediated disease, condition, or disorder comprising the administration of a pharmaceutically effective amount of a composition comprising excipients and at least one vehicle-conjugated B1 peptide antagonist of the present invention or one vehicle-conjugated B1 peptide antagonist produced using the reagents and methods of the present invention.

The novel vehicle conjugated B1 peptide antagonists of the present invention and the vehicle conjugated B1 peptide antagonists produced using the reagents and methods of the present invention may be used for the treatment or prevention of a broad spectrum of B1 mediated diseases, conditions or disorders including, but not limited to, cancer and the diseases, conditions, or disorders set forth in U.S. Application '236, including, but not limited to, inflammation and chronic pain states of inflammatory and neuropathic origin, septic shock, arthritis, osteoarthritis, angina, cancer, asthma, allergic rhinitis, and migraine.

The vehicle conjugated B1 peptide antagonists of the present invention or the vehicle-conjugated B1 peptides produced using the reagents and methods of the present invention may be used for the treatment or prevention of the diseases, conditions, and/or conditions described above or below by formulating them with appropriate pharmaceutical carrier materials known in the art and administering an effective amount of the composition to a patient, such as a human (or other mammal) in need thereof.

These and other aspects of the invention will be apparent from the consideration of the following figures and detailed description.

### DESCRIPTION OF THE DRAWINGS

FIG. 1 depicts Conjugate 28.

FIG. 2 depicts ¹H NMR spectrum (D₂O, 298K) of Conjugate 28 with both HOD and PEG signals suppressed by spin-diffusion filter and weak presaturation respectively.

FIG. 3 depicts ¹3C and ¹H NMR correlation of PEG resonances to N-terminal glycine of peptide 26 through a (9bS)-2,3-dihydrothiazolo[2,3-a]isoindol-5(9bH)-one ring.

FIG. 4 shows molecular mechanics calculations for the trans- and cis-diastereomers. The relative stereochemistry for residue H₁ was determined to be trans-relative to H₄ based on the 2D NOESY experiment (500 ms mixing time) and short (100 ps) MD runs. The calculated distances for both the cis- and trans-diastereomers, is given in table 2, along with the measured distances based on 2D NOESY. Specifically, the H₁-H₄ distance for the trans-configuration was predicted to be 4.1 Å, while the alternative cis-diastereomer would be significantly shorter (3.1 Å). The measured distance of 4.4 Å agrees well with the proposed trans-diastereomer.

FIG. 5 depicts deconvoluted FT-MS spectra for Conjugate 28.

FIG. 6 depicts the ion isolation (n=420) and IRMPD dissociation for Conjugate 28.

FIG. 7 depicts the IRMPD fragment assignment for Conjugate 28.

FIG. 8 depicts assigned resonances for Conjugate 28.

### DETAILED DESCRIPTION OF THE INVENTION

The section headings used herein are for organizational purposes only and are not to be construed as limiting the subject matter described. All documents or portions of documents cited in this application, including but not limited to patents, patent applications, articles, books, and treatises, are expressly incorporated by reference herein in their entirety for any purpose. In the event that one or more of the incorporated documents defines a term that contradicts that term's definition in this application, this application controls.

Definitions

Standard techniques may be used for recombinant DNA, oligonucleotide synthesis, and generation and identification of antibodies or antibody fragments. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, peptide syntheses, chemical analyses, chemical purification, pharmaceutical preparation, formulation, delivery, and treatment of patients.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting.

Natural amino acid residues are discussed in three ways: full name of the amino acid, standard three-letter code, or standard single-letter code in accordance with the chart shown below.

| | |
|---|---|
| A = | Ala |
| C = | Cys |
| D = | Asp |
| E = | Glu |
| F = | Phe |
| G = | Gly |
| H = | His |
| I = | Ile |
| K = | Lys |
| L = | Leu |
| M = | Met |
| N = | Asn |
| P = | Pro |
| Q = | Gln |
| R = | Arg |
| S = | Ser |
| T = | Thr |
| V = | Val |
| W = | Trp |
| Y = | Tyr |

In certain embodiments, one or more unconventional amino acids may be incorporated into a polypeptide. The term “unconventional amino acid” refers to any amino acid that is not one of the twenty conventional amino acids. The term “non-naturally occurring amino acids” refers to amino acids that are not found in nature. Non-naturally occurring amino acids are a subset of unconventional amino acids. Unconventional amino acids include, but are not limited to, stereoisomers (e.g., D-amino acids) of the twenty conventional amino acids, unnatural amino acids such as α-, α-disubstituted amino acids, N-alkyl amino acids, lactic acid, homoserine, homocysteine, 4-hydroxyproline, γ-carboxyglutamate, ε-N,N,N,-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline) known in the art. In the polypeptide notation used herein, the left-hand direction is the amino terminal direction and the right-hand direction is the carboxy-terminal direction, in accordance with standard usage and convention. Unless clearly indicated otherwise, a designation herein of a natural or non-natural amino acid is intended to encompass both the D- and L-isomer of the amino acid. Additional abbreviations used herein for certain unnatural amino acids are the same as described in U.S. Pat. No. 5,834,431, PCT publication WO 98/07746, and Neugebauer, et al. (2002). Additionally, the abbreviation “Dab” and “D-Dab” is intended to refer to the L- and D-isomer of the unnatural amino acid, D-2-aminobutyric acid, respectively. The abbreviation “3′-Pal” and “D-3′-Pal” is intended to refer to the L- and D-isomer of the unnatural amino acid 3′-pyridylalanine, respectively. Also, the abbreviation “Igl” is intended to include both “Igla” and “Iglb” (α-(1-indanyl)glycine and α-(2-indanyl)glycine, respectively). Similarly, “D-Igl” is intended to include both “D-Igla” and “D-Iglb” (the D-isomers of α-(1-indanyl)glycine and α-(2-indanyl)glycine, respectively).

Preferably, when used herein, Igl is Iglb and D-Igl is D-Iglb.

The following list of various other abbreviations used throughout the specification represent the following:
ACN, MeCN—acetonitrile
APCI MS—atmospheric pressure chemical ionization mass spectra
AgNO3—silver(I)nitrate
AIBN—2,2′-azobis(2-methylpropanenitrile)
BBr3—boron tribromide
t-BDMS-Cl—tert-butyldiethylsilyl chloride
CCl4—carbontetrachloride
Cs₂CO₃—cesium carbonate
CHCl₃—chloroform
CH₂Cl₂, DCM—dichloromethane, methylene chloride
CuBr—copper bromide
Cul—copper iodide
DIBAL—diisobutylaluminum hydride
DIC —1,3-diisopropylcarbodiimide
DIEA,(iPr)₂Net
DIPEA, Hunigs
Base—diisopropylethylamine
DCE—dichloroethane
DCM—N-hydroxysuccinimide
DME—dimethoxyethane
DMF—dimethylformamide
DMAP—4-dimethylaminopyridine
DMSO—dimethylsulfoxide
DSS—trimethylsilyl-2-silapentane-5-sulfonate-d6, sodium salt
EDC—1-(3-dimethylaminopropyl)-3 ethylcarbodiimide
Et₂O—diethyl ether
EtOAc—ethyl acetate
FBS—fetal bovine serum
FT MS—fourier transform mass spectrometry
G, gm, g—gram
h, hr—hour
H₂—hydrogen
HATU—O-(7-azabenzotriazol-1-yl)-N,N,N′,N′-tetramethyluroniumhexafluoro-phosphate
HBr—hydrobromic acid
HCl—hydrochloric acid
HOBt—1-hydroxybenzotriazole hydrate
HPLC—high pressure liquid chromatography
HRMS—High resolution mass spectrometry
IPA, i-PrOH—isopropyl alcohol
K₂CO₃—potassium carbonate
KI—potassium iodide
LiCl—lithium chloride
LiOH—lithium hydroxide
MgSO₄—magnesium sulfate
MeOH—methanol
MW—molecular weight
MWCO—molecular weight cut-off
N₂—nitrogen
NaCNBH₃—sodium cyanoborohydride
NaHCO₃—sodium bicarbonate
NaH—sodium hydride
NaOCH₃—sodium methoxide
NaOH—sodium hydroxide
Na₂SO₄—sodium sulfate
NBS—N-bromosuccinimide
NH₄Cl—ammonium chloride
NH₄OH—ammonium hydroxide
NMP—N-methylpyrrolidinone
P(t-bu)₃—tri(tert-butyl)phosphine
PBS—phosphate buffered saline
RT, rt—room temperature
TBAF—tetra-n-butylammonium fluoride
TBTU—O-benzotriazol-1-yl-N,N′,N′-tetramethyluronium tetrafluoroborate
TEA, Et₃N—triethylamine
TFA—trifluoroacetic acid
THF—tetrahydrofuran

As utilized in accordance with the present disclosure, the following terms, unless otherwise indicated, shall be understood to have the following meanings:

The term “active agent” includes within its meaning any therapeutic, bioactive and/or diagnostic agent. The term “B1” means the bradykinin B1 receptor (see, Judith M Hall, A review of BK receptors. Pharmac. Ther., 56:131-190 (1992)). Unless specifically noted otherwise, B1 or bradykinin B1 receptor is intended to mean the human bradykinin B1 receptor (hB1). Preferably, hB1 is the wild-type receptor. More preferably, hB1 is the bradykinin receptor described in GenBank Accession no. AJ238044.

The compounds of this invention may have in general several asymmetric centers and are typically depicted in the form of racemic mixtures. This invention is intended to encompass racemic mixtures, partially racemic mixtures and separate enantiomers and diasteromers.

Unless otherwise specified, the following definitions apply to terms found in the specification and claims:

“C_{α-β}alkyl” means an alkyl group comprising a minimum of α and a maximum of β carbon atoms in a branched, cyclical or linear relationship or any combination of the three, wherein α and β represent integers. The alkyl groups described in this section may also contain one or two double or triple bonds. Examples of c₆alkyl include, but are not limited to the following: 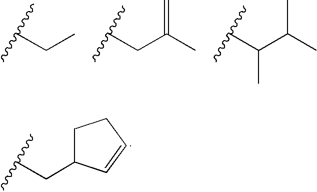
“C_{α-β}pheteroalkyl” means an a C_{α-β}alkyl wherein any of the carbon atoms of the alkyl are replaced by O, N or S. Examples of of C₁₋₆heteroalkyl include, but are not limited to the following: 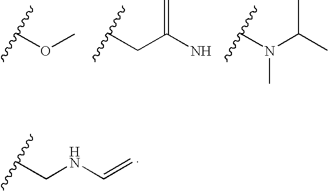
“Leaving group” generally refers to groups readily displaceable by a nucleophile, such as an amine, a thiol or an alcohol nucleophile. Such leaving groups are well known in the art. Examples of such leaving groups include, but are not limited to, N-hydroxysuccinimide, N-hydroxybenzotriazole, halides, triflates, tosylates and the like. Preferred leaving groups are indicated herein where appropriate. “Protecting group” generally refers to groups well known in the art which are used to prevent selected reactive groups, such as carboxy, amino, hydroxy, mercapto and the like, from undergoing undesired reactions, such as nucleophilic, electrophilic, oxidation, reduction and the like. Preferred protecting groups are indicated herein where appropriate. Examples of amino protecting groups include, but are not limited to, aralkyl, substituted aralkyl, cycloalkenylalkyl and substituted cycloalkenyl alkyl, allyl, substituted allyl, acyl, alkoxycarbonyl, aralkoxycarbonyl, silyl and the like. Examples of aralkyl include, but are not limited to, benzyl, ortho-methylbenzyl, trityl and benzhydryl, which can be optionally substituted with halogen, alkyl, alkoxy, hydroxy, nitro, acylamino, acyl and the like, and salts, such as phosphonium and ammonium salts. Examples of aryl groups include phenyl, naphthyl, indanyl, anthracenyl, 9-(9-phenylfluorenyl), phenanthrenyl, durenyl and the like. Examples of cycloalkenylalkyl or substituted cycloalkylenylalkyl radicals, preferably have 6-10 carbon atoms, include, but are not limited to, cyclohexenyl methyl and the like. Suitable acyl, alkoxycarbonyl and aralkoxycarbonyl groups include benzyloxycarbonyl, t-butoxycarbonyl, iso-butoxycarbonyl, benzoyl, substituted benzoyl, butyryl, acetyl, trifluoroacetyl, trichloro acetyl, phthaloyl and the like. A mixture of protecting groups can be used to protect the same amino group, such as a primary amino group can be protected by both an aralkyl group and an aralkoxycarbonyl group. Amino protecting groups can also form a heterocyclic ring with the nitrogen to which they are attached, for example, 1,2-bis(methylene)benzene, phthalimidyl, succinimidyl, maleimidyl and the like and where these heterocyclic groups can further include adjoining aryl and cycloalkyl rings. In addition, the heterocyclic groups can be mono-, di- or tri-substituted, such as nitrophthalimidyl. Amino groups may also be protected against undesired reactions, such as oxidation, through the formation of an addition salt, such as hydrochloride, toluenesulfonic acid, trifluoroacetic acid and the like. Many of the amino protecting groups are also suitable for protecting carboxy, hydroxy and mercapto groups. For example, aralkyl groups. Alkyl groups are also suitable groups for protecting hydroxy and mercapto groups, such as tert-butyl.

Silyl protecting groups are silicon atoms optionally substituted by one or more alkyl, aryl and aralkyl groups. Suitable silyl protecting groups include, but are not limited to, trimethylsilyl, triethylsilyl, triisopropylsilyl, tert-butyldimethylsilyl, dimethylphenylsilyl, 1,2-bis(dimethylsilyl)benzene, 1,2-bis(dimethylsilyl)ethane and diphenylmethylsilyl. Silylation of an amino groups provide mono- or di-silylamino groups. Silylation of aminoalcohol compounds can lead to a N,N,O-trisilyl derivative. Removal of the silyl function from a silyl ether function is readily accomplished by treatment with, for example, a metal hydroxide or ammonium fluoride reagent, either as a discrete reaction step or in situ during a reaction with the alcohol group. Suitable silylating agents are, for example, trimethylsilyl chloride, tert-butyl-dimethylsilyl chloride, phenyldimethylsilyl chloride, diphenylmethyl silyl chloride or their combination products with imidazole or DMF. Methods for silylation of amines and removal of silyl protecting groups are well known to those skilled in the art. Methods of preparation of these amine derivatives from corresponding amino acids, amino acid amides or amino acid esters are also well known to those skilled in the art of organic chemistry including amino acid/amino acid ester or aminoalcohol chemistry.

Protecting groups are removed under conditions which will not affect the remaining portion of the molecule. These methods are well known in the art and include acid hydrolysis, hydrogenolysis and the like. A preferred method involves removal of a protecting group, such as removal of a benzyloxycarbonyl group by hydrogenolysis utilizing palladium on carbon in a suitable solvent system such as an alcohol, acetic acid, and the like or mixtures thereof. A t-butoxycarbonyl protecting group can be removed utilizing an inorganic or organic acid, such as HCl or trifluoroacetic acid, in a suitable solvent system, such as dioxane or methylene chloride. The resulting amino salt can readily be neutralized to yield the free amine. Carboxy protecting group, such as methyl, ethyl, benzyl, tert-butyl, 4-methoxyphenylmethyl and the like, can be removed under hydrolysis and hydrogenolysis conditions well known to those skilled in the art.

It should be noted that compounds of the invention may contain groups that may exist in tautomeric forms, such as cyclic and acyclic amidine and guanidine groups, heteroatom substituted heteroaryl groups (Y′═O, S, NR), and the like, which are illustrated in the following examples: 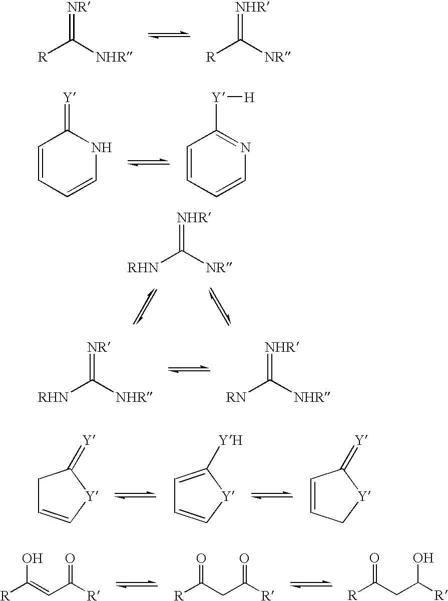
and though one form is named, described, displayed and/or claimed herein, all the tautomeric forms are intended to be inherently included in such name, description, display and/or claim.

Prodrugs of the compounds of this invention are also contemplated by this invention. A prodrug is an active or inactive compound that is modified chemically through in vivo physiological action, such as hydrolysis, metabolism and the like, into a compound of this invention following administration of the prodrug to a patient. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups as needed.

The term “diagnostic agent” includes within its meaning any compound, composition or particle which may be used in connection with methods for detecting the presence or absence of a particular agent, measuring the quantity of a particular agent, and/or imaging a particular agent, in vivo or in vitro.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is linked to a polynucleotide which it is not linked to in nature, or (3) does not occur in nature as part of a larger sequence.

The term “polymer” means a chemical compound consisting of repeating non-peptide structural units. In some embodiments of the present invention, the vehicle may be a water-soluble polymer such as PEG and methoxypolyethylene glycol (mPEG).

The terms “polynucleotide” and “oligonucleotide” are used interchangeably, and as referred to herein mean a polymeric form of nucleotides of at least 10 bases in length. In certain embodiments, the bases may comprise at least one of ribonucleotides, deoxyribonucleotides, and a modified form of either type of nucleotide. The term includes single and double stranded forms of DNA.

The term “naturally occurring nucleotides” includes deoxyribonucleotides and ribonucleotides. Deoxyribonucleotides include, but are not limited to, adenosine, guanine, cytosine, and thymidine. Ribonucleotides include, but are not limited to, adenosine, cytosine, thymidine, and uracil. The term “modified nucleotides” includes, but is not limited to, nucleotides with modified or substituted sugar groups and the like. The term “polynucleotide linkages” includes, but is not limited to, polynucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). In certain embodiments, a polynucleotide can include a label for detection.

The term “purified” when used with respect to a polypeptide, peptide or protein shall mean a polypeptide, peptide and protein which is essentially free, that is, contains less than about 50%, preferably less than about 70%, and more preferably, less than about 90% of cellular components with which that molecule of interest is naturally associated. Methods for purifying polypeptides, peptides, and proteins are well known in the art.

The terms “polypeptide,” “peptide,” and “protein” each refer to a polymer of two or more amino acids joined to each other by peptide bonds or modified peptide bonds, i.e., peptide isosteres. The terms apply to amino acid polymers containing naturally occurring amino acids as well as amino acid polymers in which one or more amino acid residues is a non-naturally occurring amino acid or a chemical analogue of a naturally occurring amino acid. A polypeptide, peptide, or protein may contain one or more amino acid residues that has been modified by one or more natural processes, such as post-translational processing such as, glycosylations, acetylations, phosphorylations and the like, and/or one or more amino acid residues that has been modified by one or more chemical modification techniques known in the art.

A “fragment” of a reference polypeptide refers to a contiguous stretch of amino acids from any portion of the reference polypeptide. A fragment may be of any length that is less than the length of the reference polypeptide.

All polypeptide, peptide, and protein sequences are written according to the generally accepted convention whereby the N-terminal amino acid residue is on the left and the C-terminal is on the right. As used herein, the term “N-terminal” refers to the free alpha-amino group of an amino acid in a peptide, and the term “C-terminal” refers to the free alpha-carboxylic acid terminus of an amino acid in a polypeptide, peptide, and protein.

The term “selective” as used herein to describe a chemical reaction between the active agent and vehicle or activated vehicle refers to a chemical reaction that will proceed in a defined and known manner such that i) other functional groups including, but not limited to, free amines, amines, guanidines, hydroxyls and carboxylic acids need not be protected and ii) the desired conjugates account for at least 50% of the reaction products.

A “variant” of a reference polypeptide refers to a polypeptide having one or more amino acid substitutions, deletions, or insertions relative to the reference polypeptide. In certain embodiments, a variant of a reference polypeptide has an altered post-translational modification site (i.e., a glycosylation site). In certain embodiments, both a reference polypeptide and a variant of a reference polypeptide are specific binding agents. In certain embodiments, both a reference polypeptide and a variant of a reference polypeptide are antibodies.

Variants of a reference polypeptide include, but are not limited to, cysteine variants. In certain embodiments, cysteine variants include variants in which one or more cysteine residues of the reference polypeptide are replaced by one or more non-cysteine residues; and/or one or more non-cysteine residues of the reference polypeptide are replaced by one or more cysteine residues. In certain embodiments, cysteine variants have more cysteine residues than the native protein.

A “derivative” of a reference polypeptide refers to: a polypeptide: (1) having one or more modifications of one or more amino acid residues of the reference polypeptide; and/or (2) in which one or more peptidyl linkages has been replaced with one or more non-peptidyl linkages; and/or (3) in which the N-terminus and/or the C-terminus has been modified; and/or (4) in which a side chain group has been modified. Certain exemplary modifications include, but are not limited to, acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. In certain embodiments, both a reference polypeptide and a derivative of a reference polypeptide are specific binding agents. In certain embodiments, both a reference polypeptide and a derivative of a reference polypeptide are antibodies. Polypeptides include, but are not limited to, amino acid sequences modified either by natural processes, such as post-translational processing, or by chemical modification techniques that are well known in the art. In certain embodiments, modifications may occur anywhere in a polypeptide, including the peptide backbone, the amino acid side-chains and the amino or carboxyl termini. In certain such embodiments, the modifications may be present to the same or varying degrees at several sites in a given polypeptide. In certain embodiments, a given polypeptide contains many types of modifications such as deletions, additions, and/or substitutions of one or more amino acids of a native sequence. In certain embodiments, polypeptides may be branched and/or cyclic. Cyclic, branched and branched cyclic polypeptides may result from post-translational natural processes (including, but not limited to, ubiquitination) or may be made by synthetic methods.

The term “biologically active” or “bioactive” means that an agent so described is capable of exerting and/or inducing a biological effect on interaction with a biological molecule or a biological system such as a polypeptide, cell or organism, in vitro or in vivo. Ways of demonstrating biological activity include in vitro bioassays, many of which are well known in the art. Biologically-active agents include, but are not limited to, therapeutic agents. The term “therapeutic agent” includes within its meaning any substance, composition or particle which may be used in any therapeutic application, such as in methods for the treatment of a disease in a patient. Therapeutic agents thus include any compound or material capable of being used in the treatment (including prevention, alleviation, pain relief or cure) of any pathological status in a patient (including, but not limited to, malady, affliction, condition, disease, disorder, lesion, trauma or injury). Non-limiting examples of therapeutic agents include pharmaceuticals, vitamins such as biotin, pantothenate, vitamin B6, and vitamin B12, nutrients, nucleic acids, such as anti-sense oligonucleotides and short interfering RNA (siRNA) molecules, amino acids, polypeptides, peptides, retro inverso (RI) and formyl-methionyl peptides, enzymes, hormones, growth factors, chemokines, antibodies and fragments thereof, enzyme co-factors, steroids, carbohydrates, lipids, organic species such as heparin, metal containing agents, receptor agonists, receptor antagonists, binding proteins, receptors or portions of receptors, extracellular matrix proteins, cell surface molecules, adhesion molecules, antigens, haptens, targeting groups, and chelating agents. All references to receptors include all forms of the receptor whenever more than a single form exists.

Additional non-limiting examples of therapeutic agents include insulin, anti-HIV peptides such as Tat inhibitor (see below), growth hormone, interferon, immunoglobulin, parathyroid hormone, calcitonin, enkephalin, endorphin, drugs, pharmaceuticals, cytotoxic agents, chemotherapy agents, radiotherapeutic agents, proteins, natural or synthetic peptides, including oligopeptides and polypeptides, vitamins, steroids and genetic material, including nucleosides, nucleotides, oligonucleotides, polynucleotides and plasmides. Among these, drugs or pharmaceuticals are preferred. Examples of drugs or pharmaceuticals include antiulcerants such as cimetidine, famotidine, ranitidine, roxatidine acetate, pantoprazole, omeprazole, lansoprazole or sucralfate; gut relaxants or prokinetics such as propantheline bromide, camylofin (acamylophenine), dicyclomine, hyoscine butyl bromide, mebeverine, cisapride, oxybutynin, pipenzolate methyl bromide, drotaverine, metoclopramide, clidinium bromide, isopropamide or oxyphenonium bromide; enzymes or carminatives, such as pancreatin, papain, pepsin, or amylase; hepatobiliary preparations such as chenodeoxycholic acid, ursodeoxycholic acid, L-ornithine or silymarin; antihypertensives such as clonidine, methyldopa sodium nitroprusside, terazosin, doxazosin, (DI) hydralazine or prazosin; beta blockers such as esmolol, celiprolol, atenolol, labetolol, propranolol, metoprolol, carvedilol, sotalol, oxyprenolol or bisoprolol; calcium channel blockers such as felodipine, nitrendipine, nifedipine, benidipine, verapamil, amlodipine or lacidipine; ace inhibitors such as enalapril, lisinopril, ramipril, perindopril, benazepril or captopril; angiotensin II inhibitors such as losartan potassium; potassium channel activators, such as nicorandil; diuretics and antidiuretics such as hydrochlorothiazide, xipamide, bumetanide, amiloride, spironolactone, indapamide, triamterene, clopamide, furosemide or chlorthalidone; antianginals such as isoscorbide dinitrate, oxyfedrine, isosorbide 5-mononitrate, diltiazem, erythrityl tetranitrate, trimetazidine, lidoflazine, pentaerythritol tetranitrate, glyceryl trinitrate or dilazep; coagulants such as conjugated oestrogens, diosmin, menaphthone, menadione, haemocoagulase, ethamsylate (cyclonamine), rutin-flavonoids or adrenochrome monosemicarbazone; anticoagulants antithrombotics or antiplatelets such as ticlopidine, warfarin, streptokinase, phenindione, rtpa, urokinase, vasopressin, nicoumalone, heparin, low molecular weight heparins, mucopolysaccharide polysulphate or dipyridamole; antiarrhythmics such as quinidine, disopyramide, procainamide, lignocaine (lidocaine), mexiletine, arniodarone, adenosine propafenone; drugs in cardiac failure and shock such as mephentermine, digoxin dopamine, dobutamine or noradrenaline, vasodilators such as isoxsuprine, xanthinol nicotinate, nylidrin HCI, pentoxifylline (oxpentifylline) or cyclandelate; cardiac glycosides such as deslaneside, digitoxin, digoxin or digitalin; penicillins such as benzyl penicillin, procaine penicillin (G), benzathine penicillin (G), phenoxymethyl penicillin, penicillin G/V, bacampicillin, carbenicillin, piperacillin, ampicillin, cloxacillin, or amoxycillin; quinolones or fluoroquinolones such as nalidixic acid, pefloxacin, ofloxacin, sparfloxacin, norfloxacin, ciprofloxacin, lomefloxacin, cephalosporins such as ceftizoxime, cefuroxime, cefixime, cefotaxime, cefaclor, ceftriaxone sodium, cefadroxil, cephalexin, cefazolin, cephaloridine, ceftazidine or ceforperazone; sulphonamides such as sulphonamides, sulphamoxole, sulphadimehtoxine, cotrifamole, cotrimoxazole, trimethoprim, aminoglycosides such as gentamicin, tobramycin, neomycin, amikacin, sisomicin, kanamycin, netilmicin, polymyxins such as polymyxin-b, colistin sulphate; chloramphenicol; tetracyclines such as tetracycline, doxycycline, minocycline, demeclocycline, oxytetracycline; macrolides such as erythromycin, clarithromycin, vancomycin, lincomycin, azithromycin, spiramycin, roxithromycin, clindamycin, cefpirome, teicoplanin (teichomycin a2), antivirals, such as abacavir, lamivudine, acyclovir, amantadine, interferon, ribavirin, stavurdine, lamivudine or zidovudine (AZT); antimalarials, such as quinine, proguanil, chloroquine, primaquine, anodiaquine, artemether, artesunate, mefloquine, pyrimethamine, arteether, mepacrine; antituberculars such as cycloserine, capreomycin, ethionamide, prothionamide, rifampicin, isoniazide, pyrazinamide, ethambutol; ethambutol, streptomycin, pyrazinamide; anthelmintics & antiinfectives such as piperazine, niclosamide, pyrantel pamoate, levamisole, diethyl carbamazine, tetramisole, albendazole, praziquantel, sodium antimony gluconate or menbendazole; antileprotics such as dapsone or clofazimine; antianaerobics, antiprotozoals or antiamoebics such as tinidazole, metronidazole, diloxanide furoate, secnidazole, hydroxyquinolones, dehydroemetine, omidazole, furazolidone; antifungals such as fluconazole, ketoconazole, hamycin, terbinafine, econazole, amphotericin-B, nystatin, clotrimazole, griseofulvin, miconazole or itraconazole; vitamins; respiratory stimulants such as doxapram hydrochloride; antiasthmatics such as isoprenaline, salbutamol(albuterol), orciprenaline, ephedrine, terbutaline sulphate, salmeterol, aminophylline, therophylline, beclomethasone dipropionate or fluticasone propionate; antiallergics such as terfenadine, astemizole, loratadine, clemastine, dimethindene maletate, fexofenadine hydrochloride, hydroxyzine, chlorpheniramine, azatadine maleate, methdilazine, pheniramine maleate, diphenhydramine or cetrizine; skeletal muscle relaxants such as tizanidine methocarbamol, carisoprodol, valethamate, baclofen, chlormezanone or chlorzoxazone; smooth muscle relaxants such as oxyphenonium bromide, propantheline bromide, diclomine, hyoscine buytyl bromide, mebeverine, drotaverine, clidinium bromide, isopropamide or camylofin dihydrochloride; non steroidal anti-inflammatory drugs such as naproxen, mefenamic acid, nimesulide, diclofenac, tenoxicam, ibuprofen, meloxicam, aspirin, flurbiprofen, ketoprofen, ketoprolac, phenylbutazone, oxyphenbutazone, indomethacin or piroxicam; antineoplastic agents, such as nitrogen mustard compounds (e.g. cyclophosphamide, trofosfamide, iofosfamide, melphalan or chlorambucil), aziridines (e.g. thioepa), N-nitrosurea derivatives (e.g. carmustine, lomustine or nimustine), platinum compounds (e.g. spiroplatin, cisplatin, and carboplatin), procarbazine, dacarbazine methotrexate, adriamycin, mitomycin, ansamitocin, cytosine arabinoside, arabinosyl adenine, mercaptopolylysine, vineristine, busulfan, chlorambucil, melphalan (e.g. PAM, L-PAM or phenylalanine mustard), mercaptopurine, mitotane, procarbazine hydrochloride dactinomycin (actinomycin D), daunorubicin hydrochloride, doxorubicin hydrochloride, epirubicin, plicamycin (mithramycin), mitoxantrone, bleomycin, bleomycin sulfate, aminoglutethimide, estramustine phosphate sodium, flutamide, leuprolide acetate, megestrol acetate, tamoxifen citrate, testolactone, trilostane, amsacrine (m-AMSA), asparaginase (L-aspar-aginase) Erwina asparaginase, etoposide (VP-16), interferons including, but not limited to, interferon a-2a, interferon a-2b, teniposide (VM-26), vinblastine sulfate (VLB), vincristine sulfate, vindesine, paclitaxel (Taxol), methotrexate, adriamycin, arabinosyl, hydroxyurea; folic acid antagonists (e.g. aminopterin, methotrexate), antagonists of purine and pyrimidine bases (e.g., mercaptopurine, tioguanine, fluorouracil or cytarabine); narcotics, opiates or sedatives such as paregoric, codeine, morphine, opium, amobarbital, amobarbital sodium, aprobarbital, butobarbital sodium, chlor-al hydrate, ethchlorvynol, ethinamate, flurazepam hydrochloride, glutethimide, methotrimeprazine hydrochloride, methyprylon, midazolam hydrochloride, paraldehyde, pentobarbital, secobarbital sodium, talbutal, temazepam or triazolam; local or general anaesthetics such as bupivacaine, chloroprocaine, etidocaine, lidocaine, mepivacaine, procaine or tetracaine, droperidol, etomidate, fentanyl citrate with droperidol, ketamine hydrochloride, methohexital sodium or thiopental; neuromuscular blockers such as atracurium mesylate, gallamine triethiodide, hexafluorenium bromide, metocurine iodide, pancuronium bromide, succinylcholine chloride, tubocurarine chloride or vecuronium bromide; or therapeutics for the hormonal system, such as growth hormone, melanocyte stimulating hormone, estradiol, beclomethasone dipropionate, betamethasone, cortisone acetate, dexamethasone, flunisolide, hydrocortisone, methylprednisolone, paramethasone acetate, prednisolone, prednisone, triamcinolone, fludrocortisone acetate, adenosine deaminase, amprenavir, albumins, laronidase, interferon alfa-N3, Palonosetron HCI, human antihemophilic factors, human coagulation factor IX, alefacept, amphotericin B, testosterone, bivalirudin, darbepoetin alfa, tazarotene, bevacizumab, morphine sulfate, interferon beta-1a, coagulation factor IX, interferon beta-1b, tositumomab and I-131 tositumomab, antihemophilic factors, human growth hormones such as sumatropin, botulinum toxin type A, exenatide, alemtuzumab, hyaluronic acid, acritumomab, alglucerase, beta-glucocerebrosidase, imiglucerase, Tadalafil, clofarabine, codeine polistirex, chlorpheniramine polistirex, Haemophilus B conjugate [meningococcal conjugate], collagen, crotalidae polyvalent immune Fab, Daptomycin, hyaluronidase, CMV immune globulin IV, daunorubicin, cytarabine, doxorubicin hydrochloride, epinastine HCI, leuprolide, rasburicase, Emtricitabine, etanercept, hepatitis B antigens, epoietin alfa, cetuximab, estradiol, clindamycin, Gemifloxacin mesylate, urofollitropin, influenza viral antigen, dexmethylphenidate hydrochloride, follitropin beta, teriparatide, calcitonin, frovatriptan succinate, enfuvirtide, gallium nitrate, human somatropin, imatinib mesylate, glucagons, metformin HCl, follitropin alfa, doxercalciferol, adefovir dipivoxil, trastuzumab, hetastarch, insulins and insulin analogs, von Willebrand factor, adalimumab, perflexane, mecasermin, interferon alfacon-1, bone morphogenetic protein-2, eptifibatide, alpha-interferon, timolol, palifermin, anakinra, insulin glargine, granulocyte macrophage colony-stimulating factor, cladribine, Fosamprenavir calcium, eszopiclone, lutropin alfa, betamethasone, OspA lipoprotein, pegaptanib, methylphenidate, methyl aminoleyulinate, mitomycin, gemtuzumab ozogamicin, botulinum toxin type B, human hepatitis B immune globulin, galsulfase, memantine HCI, Cyanocobalamin, nesiritide, pegfilgrastim, oprelvekin, Filgrastim, Technetium [99m Tc] fanolesomab, mitoxantrone, insulin aspart, coagulation factor VIIa, clobetasol proprionate, L-asparaginase, denileukin diftitox, amlexanox, nitisinone, muromomab-CD3, human chorionic gonadotropin, Bacillus Calmette-Guerin antigens, alitretinoin, diphtheria, peginterferon alfa-2a, porfimer sodium, gonadotropin-releasing hormone antagonists, repaglinide, pneumococcal 7-valent conjugate, ziconotide, ciprofloxacin hydrochloride, indium In 111 capromab pendetide, somatrem, modafinil, domase alfa, samarium SM-153 lexidronam, omeprazole, Efalizumab, ribavirin and alpha interferon, lepirudin, gel becaplermin, infliximab, treprostinil sodium, sevelamer hydrochloride, abciximab, reteplase, Rh0 immune globulin, rituximab, interferon alfa-2a, trospium chloride, fluoxetine hydrochloride, synthetic porcine secretin, cinacalcet HCl, basiliximab, pegvisomant, pramlintide acetate, Palivizumab, oseltamivir phosphate, erlotinib (OSI Pharmaceuticals, Inc. and Genentech), bexarotene, bexarotene, antithymocyte globulin, thyrotropin alfa, thyroglobulin (Tg), tenecteplase, flu, diphtheria, tetanus and acellular pertussis antigens, diphtheria, tetanus toxoids and acellular pertussis antigens, arsenic trioxide, emtricitabine, natalizumab, bortezomib, iloprost, azacitidine, nelfinavir, tenofovir disoproxil fumarate, cidofovir injection, verteporfin, fomivirsen, interferon alfa-n1, Rho[D] immune globulin, bromfenac sodium, rifaximin, drotrecogin alfa, Omalizumab, sodium oxybate, miglustat, omeprazole, daclizumab, ibritumomab tiuxetan, zonisamide, loteprednol etabonate, tobramycin, bromhexine, carbocysteine or clavulanic acid, docosanol, paracetamol, interferon gamma-1b, alteplase, and technetium Tc-99 apcitide.

The active agents linked to vehicles in the conjugates of the present invention have or are modified to have a 1,2- or 1,3-aminothiol moiety or a group of formula I capable of reacting with the vehicle derivatives via it's complimentary functionality as described herein prior to forming the linkage. An example of a reactive 1,2-aminothiol is found in the amino acid cysteine.

Many proteins do not have free cysteines (cysteines not involved in disulfide bonding) or any other reactive 1,2- or 1,3-aminothiol group. In addition, the cysteine 1,2-aminothiol may not be appropriate for linkage to the polymer because the 1,2-aminothiol is necessary for biological activity. In addition, proteins must be folded into a certain conformation for activity. In the active conformation, the 1,2-aminothiol of a cysteine can be inaccessible because it is buried in the interior of the protein. Moreover, even an accessible cysteine 1,2-aminothiol which is not necessary for activity can be an inappropriate site to form a linkage to the polymer. Amino acids not essential for activity are termed “nonessential”. Nonessential cysteines can be inappropriate conjugation sites because the cysteine's position relative to the active site results in the polypeptide becoming inactive after conjugation to a vehicle.

Like proteins, many other biologically-active molecules have reactive 1,2- or 1,3-aminothiol which, for reasons similar to those recited above, are not suitable for conjugation to a particular vehicle or contain no reactive 1,2- or 1,3-aminothiol groups. Accordingly, the present invention contemplates the introduction of reactive 1,2- or 1,3-aminothiol groups into a biologically-active agent when necessary or desirable, which may be conjugated to a vehicle derivative of the present invention. Examples of thioamide-moiety-containing biologically active agents are described in U.S. patent application Ser. No. 09/621,109. Such compounds include but are not limited to UC781; R82150; HBY097; troviridine; S2720; UC38 and 2′,3′-dideoxy-3′-fluoro-4-thiothymidine.

Reactive thiol groups or thioamide groups can be introduced by chemical means well known in the art. Chemical modification can be used with polypeptides or non-peptidic molecules and includes the introduction of thiol alone or as part of a larger group, for example a cysteine residue, into the molecule. One can also generate a free cysteine in a polypeptide by chemically reducing cysteine with, for example, DTT.

Polypeptides which are modified to contain an amino acid residue in a position where one was not present in the native protein before modification is called a “mutein.” To create cysteine muteins, a N-terminial nonessential amino acid can be substituted with a cysteine. The mutation of an N-terminal lysine to cysteine is also appropriate because lysine residues are often found on the surface of a protein in its active conformation. In addition, one skilled in the art can use any information known about the binding or active site of the polypeptide in the selection of possible mutation sites. One skilled in the art can also use well-known recombinant DNA techniques to create cysteine muteins. One can alter the nucleic acid encoding the native polypeptide to encode the mutein by standard site directed mutagenesis. Examples of standard mutagenesis techniques are set forth in Kunkel, T. A., Proc. Nat. Acad. Sci., Vol. 82, pp. 488-492 (1985) and Kunkel, T. A. et al., Methods Enzymol., Vol. 154, pp. 367-382 (1987).

Potential sites for introduction of a non-native cysteine include glycosylation sites and the N terminus of the polypeptide. In these examples, the glycosyl donor could contain a 1,2- or 1,3-aminothiol. One skilled in the art could attach glycosyl groups to serine or threonine on the active agent.

Alternatively, one can chemically synthesize the nucleic acid encoding the mutein by techniques well known in the art. DNA synthesizing machines can be used and are available, for example, from Applied Biosystems (Foster City, Calif.). The nucleic acid encoding the desired mutein can be expressed in a variety of expression systems, including animal, insect, and bacterial systems. After creation of the desired mutein, one skilled in the art can bioassay the mutein and compare activity of the mutein relative to the native polypeptide. Even if the relative activity of the mutein is diminished, the conjugate formed from the mutein can be particularly useful. For example, the conjugate can have increased solubility, reduced antigenicity or immunogenicity, or reduced clearance time in a biological system relative to the unconjugated molecule.

“Polypeptides” and “proteins” are used herein synonymously and mean any compound that is substantially proteinaceous in nature. However, a polypeptidic group may contain some non-peptidic elements. For example, glycosylated polypeptides or synthetically modified proteins are included within the definition.

As used herein, the terms “effective amount” and “therapeutically effective amount” when used with reference to bioactive agent such as a peptide, vehicle-conjugated peptide, or PEG-conjugated peptide refers to an amount or dosage sufficient to produce a desired result. In the context of vehicle-conjugated B1 peptides, and/or PEG-conjugated peptide B1 antagonists, the desired result may be a desired reduction in inflammation and/or pain, for example, or to support an observable decrease in the level of one or more biological activities of B1. More specifically, a therapeutically effective amount is an amount of the biologically active agent that is sufficient to reduce, inhibit, or prevent, for some period of time, one or more of the clinically defined pathological processes associated with the condition at issue, e.g., inflammation or pain, in a subject treated in vivo with the agent(s). The effective amount may vary depending on the biological agent, and is also dependent on a variety of factors and conditions related to the subject to be treated and the severity of the disorder. For example, if the biologically active conjugate is to be administered in vivo, factors such as the age, weight and health of the patient as well as dose response curves and toxicity data obtained in preclinical animal work would be among those considered. If the biologically active conjugated is to be contacted with the cells in vitro, one would also design a variety of pre-clinical in vitro studies to assess such parameters as uptake, half-life, dose, toxicity, etc. The determination of an effective amount or a therapeutically effective amount for a given agent is well within the ability of those skilled in the art.

The term “pharmacologically active” means that a substance so described is determined to have activity that affects a medical parameter or disease state (for example, pain). In the context of the vehicle-conjugated B1 peptides of the present invention, this term typically refers to a B1-induced or B1-mediated disease, disorders, or abnormal medical conditions and more specifically, to antagonism of inflammation or pain.

The terms “antagonist”, “inhibitor”, and “inverse agonist” (e.g., see, Rianne A. F. de Ligt, et. al, British Journal of Pharmacology 2000, 130, 131) refer to a molecule that blocks, impedes, reduces, lessens or in some way interferes with the biological activity of the associated protein of interest. A preferred “B1 peptide antagonist” of the present invention is a molecule that binds to and inhibits B1 with an IC₅₀ of 500 nM or less in in vitro assays of B1 activity. A more preferred B1 peptide antagonist of the present invention is a molecule that binds to the receptor with a Ki of 100 nM or less and inhibits a B1 mediated functions, such as calcium flux, with an IC₅₀ less than 100 nM in in vitro assays of B1 activity. A most preferred B1 peptide antagonist of the present invention is a molecule that binds to and inhibits B1 with a Ki of less than 10 nM and an IC₅₀ of 10 nM or less in in vitro assays of B1 activity. Furthermore, said molecule would prevent, ameliorate or abolishe pain or inflammation as measured in at least one generally accepted in vivo animal model of pain and/or inhibits biochemical challenges in in vivo animal models of edema, inflammation, or pain.

Additionally, physiologically acceptable salts of the peptides or conjugated peptides of the invention are also encompassed herein. The phrases “physiologically acceptable salts” and “pharmacologically acceptable salts” as used herein are interchangeable are intended to include any salts that are known or later discovered to be pharmaceutically acceptable (i.e., useful in the treatment of a warm-blooded animal). Some specific examples are: acetate; hydrohalides, such as hydrochloride and hydrobromide; sulfate; citrate; tartrate; glycolate; oxalate; salts of inorganic and organic acids, including, but not limited to, hydrochloric acid, hydrobromic acid, sulfuric acid, phosphoric acid, methanesulphonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al., J. Pharm. Sci. 66:1 (1977).

“Protecting group” generally refers to groups well known in the art which 10 are used to prevent selected reactive groups, such as carboxy-, amino-, hydroxyl-, mercapto- and the like, from undergoing undesired reactions, such as nucleophilic, electrophilic, oxidation, reduction and the like. Preferred protecting groups are indicated herein where appropriate. Examples of amino protecting groups include, but are not limited to, arylalkyl-, substituted arylalkyl-, cycloalkenylalkyl- and substituted cycloalkenyl- alkyl-, allyl-, substituted allyl-, acyl-, alkoxycarbonyl-, arylalkoxycarbonyl-, silyl- and the like. Examples of arylalkyl- include, but are not limited to, benzyl-, ortho-methylbenzyl-, trityl- and benzhydryl-, which can be optionally substituted with halogen, alkyl-, alkoxy-, hydroxyl-, nitro-, acylamino-, acyl- and the like, and salts, such as phosphonium and ammonium salts. Examples of aryl groups include phenyl-, naphthyl-, indanyl-, anthracenyl-, 9-(9-phenylfluorenyl)-, phenanthrenyl-, durenyl- and the like. Examples of cycloalkenylalkyl- or substituted cycloalkylenylalkyl-radicals, preferably have 6-10 carbon atoms, include, but are not limited to, cyclohexenyl-, methyl- and the like. Suitable acyl-, alkoxycarbonyl- and aralkoxycarbonyl-groups include benzyloxycarbonyl-, t-butoxycarbonyl-, iso-butoxycarbonyl-, benzoyl-, substituted benzoyl-, butyryl-, acetyl-, trifluoroacetyl-, trichloroacetyl-, phthaloyl- and the like. A mixture of protecting groups can be used to protect the same amino group, such as a primary amino group can be protected by both an arylalkyl-group and an arylalkoxycarbonyl-group. Amino protecting groups can also form a heterocyclic ring with the nitrogen to which they are attached, for example, 1,2-bis(methylene)-benzene, phthalimidyl-, succinimidyl-, maleimidyl- and the like and where these heterocyclic groups can further include adjoining aryl- and cycloalkyl-rings. In addition, the heterocyclic groups can be mono-, di- or tri-substituted, such as nitrophthalimidyl-. Amino groups may also be protected against undesired reactions, such as oxidation, through the formation of an addition salt, such as hydrochloride, toluenesulfonic acid, trifluoroacetic acid and the like. Many of the amino protecting groups are also suitable for protecting carboxy-, hydroxyl- and mercapto-groups. For example, arylalkyl-groups. Alkyl groups are also suitable groups for protecting hydroxyl- and mercapto-groups, such as tert-butyl.

Silyl-protecting groups are silicon atoms optionally substituted by one or more alky-1, ary-1 and arylalkyl-groups. Suitable silyl protecting groups include, but are not limited to, trimethyl-silyl, triethylsilyl, tri-isopropylsilyl, tert-butyldimethylsilyl, dimethylphenylsilyl, 1,2-bis(dimethylsilyl)benzene, 1,2-bis(dimethylsilyl)-ethane and diphenylmethylsilyl. Silylation of an amino groups provide mono- or di-silylamino groups. Silylation of aminoalcohol compounds can lead to a N,N,O-tri-silyl derivative. Removal of the silyl function from a silyl ether function is readily accomplished by treatment with, for example, a metal hydroxide or ammonium fluoride reagent, either as a discrete reaction step or in situ during a reaction with the alcohol group. Suitable silylating agents are, for example, trimethylsilyl chloride, tert-butyl-dimethylsilyl chloride, phenyldimethylsilyl chloride, diphenylmethyl silyl chloride or their combination products with imidazole or DMF. Methods for silylation of amines and removal of silyl protecting groups are well known to those skilled in the art. Methods of preparation of these amine derivatives from corresponding amino acids, amino acid amides or amino acid esters are also well known to those skilled in the art of organic chemistry including amino acid/amino acid ester or aminoalcohol chemistry.

Protecting groups are removed under conditions that will not affect the remaining portion of the molecule. These methods are well known in the art and include acid hydrolysis, hydrogenolysis and the like. A preferred method involves removal of a protecting group, such as removal of a benzyloxycarbonyl group by hydrogenolysis utilizing palladium on carbon in a suitable solvent system such as an alcohol, acetic acid, and the like or mixtures thereof. A t-butoxy-carbonyl protecting group can be removed utilizing an inorganic or organic acid, such as HCl or trifluoroacetic acid, in a suitable solvent system, such as dioxane or methylene chloride. The resulting amino salt can readily be neutralized to yield the free amine. Carboxy protecting group, such as methyl, ethyl, benzyl, tert-butyl, 4-methoxyphenylmethyl and the like, can be removed under hydrolysis and hydrogenolysis conditions well known to those skilled in the art. A more comprehensive use of protecting groups is described in Theodora W. Green and Peter G. M. Wuts (1999), “Protective Groups in Organic Synthesis”, Third Edition, Wiley, New York, N.Y.

The present invention is based upon the identification of a novel chemical process that provides novel vehicle derivatives that are exceptional 1,2- or 1,3-aminothiol selective reagents for conjugating to unprotected targeted agents (e.g., polypeptides, peptides, or organic compounds) having or modified to have a 1,2- or 1,3 aminothiol group. The extraordinarily specific reaction regioselectively forms a covalent bond between the vehicle derivative and a 1,2- or 1,3-aminothiol moiety of the targeted active agent. The reaction proceeds almost entirely to completion under very mild conditions.

Although the synthesis of proteins by the chemoselective reaction of a cysteine-containing fragment with an aldehyde-containing fragment has been described (Liu, C.-F.; Tam, J. P. J. Am. Chem. Soc. 1994, 116,.4149. Liu, C.-F.; Rao, C.; Tam, J. P. J. Am. Chem. Soc. 1996, 118,.307; Tam, J. P.; Miao, Z. J. Am. Chem. Soc. 1999, 121,.9013. Melnyk, O.; Fruchart, J.-S.; Grandjean, C.; Gras-Masse, H. J. Org. Chem. 2001, 66, 4153), the chemical ligation approaches described herein have not been applied as a method for conjugating peptides, proteins, or organic compounds to vehicles.

In one embodiment the present invention relies on the unique ability of a 1,2- or 1,3-aminothiol to chemoselectively react with an aldehyde to form a thiazoline. Once formed, the thiazoline nitrogen is kinetically predisposed to form an amide bond. This is accomplished by the placement of an ester carbonyl 5- or 6-atoms removed from the thiazoline nitrogen. In addition, the novel chemical reactions of the present invention generally results in a single predominant species facilitating ease of purification, analysis, and characterization of the desired conjugate.

The novel chemical reagents and processes of the present invention are particularly effective in strategies for the generation of multi-peptide vehicle conjugates. For example, the reagents and methods of the present invention were used to efficiently conjugate four cysteine containing B1 peptide antagonists onto a branched multivalent PEG polymer. The reagents and methods described herein efficiently generated the desired multi-peptide PEG conjugates in high yields and high purity. Various multi-peptide PEG conjugates demonstrated increased activity (hB1 Ki=100 pm, in some cases), dramatically longer circulating half-lives, decreased PEG load allowing for acceptable dosing regimens that provide significantly greater exposure and prolonged efficacy in vivo when compared to peptide conjugates having a single peptide per vehicle. Vehicle-conjugated B1 peptides provide tremendous therapeutic advantage over known unconjugated B1 peptide antagonists and may be useful for the treatment and/or prevention of B1 mediated diseases, conditions, or disorders, including, but not limited to, inflammation and pain.

The use of the novel activated vehicle derivatives of the present invention in the methods of the present invention resulted in numerous surprising and unexpected advantages over previously known polymer conjugation methodologies, especially with respect to multi-valent polymer conjugation strategies (see, for example, PCT publication WO 95/06058, U.S. Patent Application Publication US 2003/0040127).

It is to be understood that the terminology used herein is for the purpose of describing particular embodiments only, and is not intended to be limiting since the scope of the present invention will be limited only by the appended claims.

Bradykinin B1 receptor binding peptides contemplated for conjugation to a vehicle for purposes and in the manner as described herein include, but are not limited to, the novel B1 binding peptide antagonists disclosed herein as well as B1 peptide antagonists known in the art including, but not limited, to any peptide disclosed in any one of the following publications (each of which is hereby incorporated by reference in its entirety): Regoli et al., Bradykinin receptors and their antagonists. Eur. J. of Pharma., 348:1-10 (1998); Neugebauer, W., et al., Kinin B₁ receptor antagonists with multi-enzymatic resistance properties. Can. J. Physiol. Pharmacol., 80:287-292 (2002); Stewart, J. M., et al, Bradykinin antagonists: present progress and future prospects. Immunopharmacology, 43:155-161 (1999); Stewart, J. M., et al., Metabolism-Resistant Bradykinin Antagonists: Development and Applications. Biol. Chem., 382:37-41 (2001); PCT Publications WO 98/07746 and WO 2005042027; and U.S. Pat. Nos. 4,693,993, 4,801,613, 4,923,963, 5,648,336, 5,834,431, 5,849,863, 5,935,932, 5,648,333, 5,385,889, 5,444,048, and 5,541,286.

A “functionalizing reagent” according to the present invention is a reagent adapted for functionalizing a vehicle according to the present invention.

A “functionalizing reaction” is a reaction in which a vehicle is functionalized according to the present invention. A functionalizing reaction can consist of one or more stages.

The term “vehicle” as used herein refers to a molecule that slows degradation, increases half-life, reduces toxicity, reduces immunogenicity, and/or increases biological activity of an active agent. Vehicles useful in the context of the present invention are known in the art and include, but are not limited to, an Fc domain, polyethylene glycol, and dextran. Various vehicles are described, e.g., in U.S. Pat. No. 6,660,843, published PCT Application Nos. WO 99/25044 and WO 98/07746, Langer, R., “Biomaterials in Drug Delivery,” 33 ACC. CHEM. RES. 94 (2000); and Langer, R., “Tissue Engineering,” 1 MOL. THER. 12 (2000), Haisch, A. et al., Tissue Engineering of Human Cartilage Tissue, 44 HNO 624 (1996); Ershov, I. A. et al., Polymer Biocompatible X-Ray Contract Hydrogel, 2 MED. TEKH. 37 (1994); Polous, I. M. et al., Use of A Biocompatible 25 Antimicrobial Polymer Film, 134 VESTN. KHIR. IM. II GREK. 55 (1985). Additional examples of vehicles include N-vinylpyrrolidone-methyl methacrylate co-polymer, perhaps with added polyamide-6 (Buron, F. et al., Biocompatable Osteoconductive Polymer, 16 CLIN. MATER. 217 (1994)), poly(DL-lactide-co-glycolide) (Isobe, M. et al., Bone Morphogenic Protein Encapsulated with a Biodegradable and Biocompatible Polymer, 32 J. BIOMED. MATER. RES. 433 (1996)), a 70:30 ratio mixture of methylmeth-acrylate:2-hydroxyethyl methacrylate (Bar, F. W. et al., New Biocompatable Polymer Surface Coating, 52 J. BIOMED. MATER. RES. 193 (2000)), 2-methacryloyl-oxyethyl phosphorylcholine, optionally with poly-urethane (Iwasaki, Y. et al., Semi-Interpenetrating Polymer Networks . . . , 52 J. BIOMED. MATER. RES. 701 (2000)), calcium alginate, such as purified high guluronic acid alginates (Becker, T. A. et al., Calcium Alginate Gel, 54 J. BIOMED. MATER. RES. 76 (2001)), protein polymers (e.g., Buchko, C. J. et al., Surface Characterization of Porous, Biocompatible Protein Polymer Thin Films, 22 BIOMATERIALS 1289 (2001); cf. Raudino, A. et al., Binding of Lipid Vescicles . . . , 231 J. COLLOID. INTERFACE SCI. 66 (2000)), polyvinyl pyrolidone, polymethylethylene-glycol, polyhydroxy-propyleneglycol, polypropylene-glycols and oxides, polymethylpropylene-glycol, poly-hydroxypropyleneoxide, straight-chain and branched-chain polypropyleneglycols, polyethyleneglycol and polypropyleneglycol and the monomethyl ethers, monocetyl ethers, mono-n-butyl ethers, mono-t-butyl-ethers and monooleyl ethers thereof, esters of poly-alkyleneglycols with carboxylic acids and dehydration condensation products of the polyalkyleneglycols with amines and other polyalkylene oxides and glycols, poly(vinylpyrrolidone), polyvinyl alcohol, poly(vinyl acetate), the copolymer poly(vinyl acetate-co-vinyl alcohol), polyvinyloxazolidone, poly(vinylmethyl-oxazolidone) and poly(vinyl methyl ether), poly(acrylic acid)s, poly(methacrylic acid)s, polyhydroxyethyl-methacrylates, poly(acrylamide) and poly(methacrylamide), poly(N,N-dimethylacrylamide), poly(N-isopropylacrylamide), poly(N-acetamidoacryl-amide) and poly(N-acetamidomethacrylamide, and other N-substituted derivatives of the amides.

PEG is a water soluble, non-immunogenic, biocompatible material. When used as vehicle, the useful properties of PEG generally conferred to the appended agent include improved solubility, increased circulation lifetime in bloodstream, resistance to proteases and nucleases, less immunogenicity, etc. The large molecular weight of PEG makes it very easy to separate the final conjugates from excess unconjugated peptide and other small-size impurities. PEG conjugates are thus stable when stored under controlled conditions and convenient for use in diagnostic assays. While the polyether backbone of PEG is relatively chemically inert, the primary hydroxyl groups on both ends are reactive and can be utilized directly to attach reactive substances. These hydroxyl groups are routinely transformed into more reactive functional groups for conjugation purposes.

The phrases “activated vehicle derivative”, “activated vehicle”, “functionalized vehicle derivative” and “functionalized vehicle” are used interchangeably herein and are intended to mean a vehicle having a reactive group at the terminus of one at least one vehicle segment. Similarly, the phrases “activated vehicle segment” and “functionalized vehicle segment” are used interchangeably herein and are intended to mean a vehicle segment having a terminal reactive group.

PEG is a water soluble, non-immunogenic, biocompatible material. When used as vehicle, the useful properties of PEG generally conferred to the appended agent include improved solubility, increased circulation lifetime in bloodstream, resistance to proteases and nucleases, less immunogenicity, etc. The large molecular weight of PEG makes it very easy to separate the final conjugates from excess unconjugated peptide and other small-size impurities. PEG conjugates are thus stable when stored under controlled conditions and convenient for use in diagnostic assays. While the polyether backbone of PEG is relatively chemically inert, the primary hydroxyl groups on both ends are reactive and can be utilized directly to attach reactive substances. These hydroxyl groups are routinely transformed into more reactive functional groups (i.e., “activated) for conjugation purposes.

The phrases “vehicle-conjugated active agent” and “conjugated active agent” are used interchangeably herein and are intended to mean a conjugate comprising at least one active agent and a vehicle comprising at least one vehicle segment that is covalently attached to the active agent itself or to a linker (including, but not limited to, a peptidyl or non-peptidyl linker (e.g., an aromatic linker) that is covalently bound to the active agent.

In some embodiments of the present invention, “vehicle-conjugated peptide” or “conjugated peptide” refers to a conjugate comprising a peptide having or modified to have a N-terminal cysteine and a vehicle comprising a vehicle segment covalently bound to the N-terminal cysteine residue of at least one peptide. In other embodiments, the conjugate comprises at least one peptide and a vehicle comprising at least one vehicle segment that is covalently bound to a non-peptidyl linker including, but not limited to, an aromatic linker, that is covalently bound to a residue of the peptide.

In some embodiments of the present invention, “PEG-conjugated peptide” refers to a conjugate comprising at least one peptide having or modified to have a N-terminal cysteine and a PEG comprising a PEG segment covalently bound to the N-terminal cysteine residue of at least one peptide. In other embodiments, the conjugate comprises at least one peptide and a PEG comprising at least one PEG segment that is covalently bound to a non-peptidyl linker including, but not limited to, an aromatic linker, that is covalently bound to a residue of at least one peptide.

In another embodiment, in conjunction with the above and below embodiments, the conjugated peptide comprises a vehicle comprising a vehicle segment covalently bound to a N-terminal cysteine residue of a peptide selected from SEQ ID NOS: 11-23 and 43-46 further modified to have said N-terminal cysteine.

In some embodiments of the invention, the vehicle may have a nominal average molecular mass ranging from about 100 to about 200,000 daltons, or a nominal average molecular mass ranging from about 100 to about 100,000 daltons, or a nominal average molecular mass ranging from about 5,000 to about 100,000 daltons, or a nominal average molecular mass ranging from about 10,000 to about 60,000 Daltons, or a nominal average molecular mass ranging from about 10,000 to about 40,000 daltons, or a nominal average molecular mass ranging from about 20,000 to about 40,000 daltons.

The reactive group on an activated vehicle may be any of a number of moieties that can participate in a reaction that can bind the various components of a desired conjugate together without significant detrimental consequences. Non-limiting examples include an acid, an ester, a thiol, an amine, or a primary amine, but these are merely illustrative of the invention. Importantly, the covalent bond that forms between the vehicle or vehicle segment(s) and any of the prescribed active agent(s) conjugated thereto should be relatively non-labile.

Typically, activated vehicles are linear and therefore only have capacity for up to two functional groups (i.e., one on the each end). Obviously, this limits the number of conjugations to just two. A vehicle with multiple reactive groups for attachment of multiple active agents to the same vehicle molecule may be preferred in some situations. The methods of the present invention are very conducive to the design of conjugation strategies that provide relatively precise numbers of functional groups on a desired multivalent vehicle.

In particular embodiments of the present invention, the vehicle may be a multivalent vehicle molecule including, but not limited to, a linear vehicle activated at both termini, a forked vehicle having more than one activated vehicle segments, and a branched vehicle having more than one activated vehicle segment. In some embodiments of the present invention, the vehicle may be a multivalent PEG including, but not limited to, a linear PEG activated at both termini, a forked PEG (fPEG) having more than one activated vehicle segments, and a branched PEG (bPEG) having more than one activated vehicle segments.

In a particular embodiment of the present invention, a vehicle derivativized with an amine or a vehicle comprising multiple vehicle segments at least one of which is derivatized with an amine is reacted with a 1,2- or 1,3-formyl ester to produce a vehicle conjugate of the present invention.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

### EXAMPLES

General Experimental.

NMR: Proton NMR for PEG containing molecules were referenced to a PEG singlet (3.7 ppm relative to DSS in D₂O). ¹³C NMR spectra were referenced to the PEG singlet (72.0 ppm relative to DSS in D₂O).

FTMS data were acquired on a Bruker Q-FTMS operating at 7 tesla. The instrument was externally calibrated with a PEG300/600 solution using the standard Francel equation. The calculated mass error for each calibrant ion was less than 1.0 ppm from the measured value. For each spectra 512 k data points were collected using a 1.25 MHz sweep width of detection (86 Da mass cutoff). The time domain data were not processed prior to performing a magnitude mode Fourier transform.

GC-MS data were recorded using a Hewlett-Packard GC-Ms with the following parameters:

Column: J and W DB-XLB capillary column, 30m×0.25mm×0.50 μM, PN 1221236.

Method 1:

Injector parameters: Injector Temperature=250° C.; 50:1 split ratio; Helium flow rate=1 mL/min.

GC parameters: Initial temperature=80° C.; From 0 to 2 minutes, hold at 80° C.; from 2 to 14 minutes ramp to 200° C.; hold at 200° C. for 5 minutes. Re-equilibrate for 0.5 min.

Mass spec transfer temperature=280° C.

Mass spectra parameters: scan from 50 to 550 amu, EI voltage=2376.5 mV.

Method 2:

Injector parameters: Injector Temperature=250° C.; 50:1 split ratio; Helium flow rate=1 mL/min.

GC parameters: Initial temperature=140° C.; From 0 to 2 minutes, hold at 140° C.; from 2 to 11 minutes ramp to 320° C.; hold at 320° C. for 1 minutes. Re-equilibrate for 0.5 min.

Mass spec transfer temperature=280° C.

Mass spectra parameters: scan from 50 to 550 amu, EI voltage=2376.5 mV

Method 3:

Injector parameters: Injector Temperature=250° C.; 50:1 split ratio; Helium flow rate=1 mL/min.

GC parameters: Initial temperature=70° C.; From 0 to 2 minutes; ramp to 90° C. at 10° C. per min; ramp to 320° C. at 20° C. per min; hold at 320° C. for 4.5 minutes. Re-equilibrate for 0.5 min.

Mass spec transfer temperature=280° C.

Mass spectra parameters: scan from 50 to 550 amu, EI voltage=2376.5 mV

Peptides were synthesized using the standard FMOC strategy as describe in “Solid Phase Peptide Synthesis” by Stewart and Young (1984). A chemist skilled in the art of peptide synthesis would be able to synthesize the described peptides by manual or automatic solid phase methods.

Peptide content by HPLC with chemiluminescence detection (CLND):

Solvent system: A=0.04% TFA in water, B=0.04% TFA in 90% Methanol.

Column: Jupiter C18 300 Å, 50×2.0 mm column, 5 μm particle size.

CLND: Antek 8060, oven temperature 1048° C., the detector was run at high sensitivity and attenuation 1.

HPLC: HP1100 LC, diode array detector

Gradient: 10% B to 100% B in 10 min and hold for 2 min, re-equilibrate for 4 min.

Flow and splitting: Total flow was 0.3 ml/min, and it was split with a tee at approximately 2:1 between CLND and waste.

Preparative Reverse Phase HPLC:

System: two Agilent series 1100 prep pumps, Agilent series 1100 prep auto injector, Rheodyne manual injector with 5-20 mL sample loops, Agilent Series 1100 multi-wavelength detector (set to 215- and 254 nm) and Agilent series 1100 automatic fraction collector.

Software: Agilent Chemstation.

Solvent System:

1: A=10 mM NH4 Formate in water (pH=3.75); B=Acetonitrile.

2: A=0.1% acetic acid in water B=0.1% acetic acid in acetonitrile.

3: A=10 mM NH4 bicarbonate (pH 10) in water; B=Acetonitrile.

Columns:

1: Waters Xterra Prep C18 MS Packed by Vydac/The Separations Group, 50 mm×300 mm (PN PA0000-050730), 10 μm particle size, spherical shape.

2: 30×100 mm Waters Xterra Prep C18 OBD, 100 Å pore diameter, 5 μm particle size, spherical shape, PN 186001942.

Gradient Tables:

1

| Time (min) | % B | Flow (mL/min) |
|---|---|---|
| 0 | 25 | 20 |
| 4 | 25 | 20 |
| 5 | 25 | 100 |
| 25 | 55 | 100 |
| 35 | 55 | 100 |
| 35.1 | 100 | 100 |
| 49.9 | 100 | 100 |
| 50 | 25 | 100 |
| 60 | 25 | 100 |
| 60.1 end | | |

2

| Time (min) | % B | Flow (mL/min) |
|---|---|---|
| 0 | 25 | 35 |
| 5 | 25 | 35 |
| 20 | 55 | 35 |
| 24.9 | 55 | 35 |
| 24.95 | 100 | 35 |
| 29.9 | 100 | 35 |
| 29.95 | 25 | 35 |
| 40 end | | |

3

| Time (min) | % B | Flow (mL/min) |
|---|---|---|
| 0 | 10 | 35 |
| 5 | 10 | 35 |
| 20 | 25 | 35 |
| 24.9 | 25 | 35 |
| 24.95 | 100 | 35 |
| 29.9 | 100 | 35 |
| 29.95 | 10 | 35 |
| 40 end | | |

4

| Time (min) | % B | Flow (mL/min) |
|---|---|---|
| 0 | 70 | 20 |
| 4 | 70 | 20 |
| 5 | 70 | 100 |
| 25 | 100 | 100 |
| 35 | 100 | 100 |
| 49.9 | 100 | 100 |
| 50 | 70 | 100 |
| 60 | 70 | 100 |
| 60.1 end | | |

Preparative Cation Exchange LC:

System and software: same as describe for preparative HPLC.

Solvents:

1: A=10 mM Boric acid in 5:40:55 MeOH-Acetonitrile-water; B=A+0.2 M KCl.

Columns:

1: Tosoh Bioscience TSKGel SP-5PW-HR, PN 43382, 20 μm particle packed into a 50×250 mm glass column (Hodge Bioseparations Ltd. P/N=TAC50/250S2-SR-1). Measured bed length=180 mm.

2: 21.5×150 mm TSK Gel SP-5PW, PN 07575.

Gradient Tables:

1

| Time (min) | % B | Flow (mL/min) |
|---|---|---|
| 0 | 0 | 10 |
| 2 | 0 | 10 |
| 5 | 0 | 30 |
| 25 | 0 | 30 |
| 25.1 | 20 | 30 |
| 80 | 80 | 30 |
| 80.1 | 100 | 30 |
| 110 | 100 | 30 |
| 110.1 | 0 | 30 |
| 130 end | | |

2

| Time (min) | % B | Flow (mL/min) |
|---|---|---|
| 0 | 0 | 10 |
| 5 | 0 | 10 |
| 30 | 100 | 10 |
| 45 | 100 | 10 |
| 45.05 | 0 | 10 |
| 60 end | | |

Experimental Section.

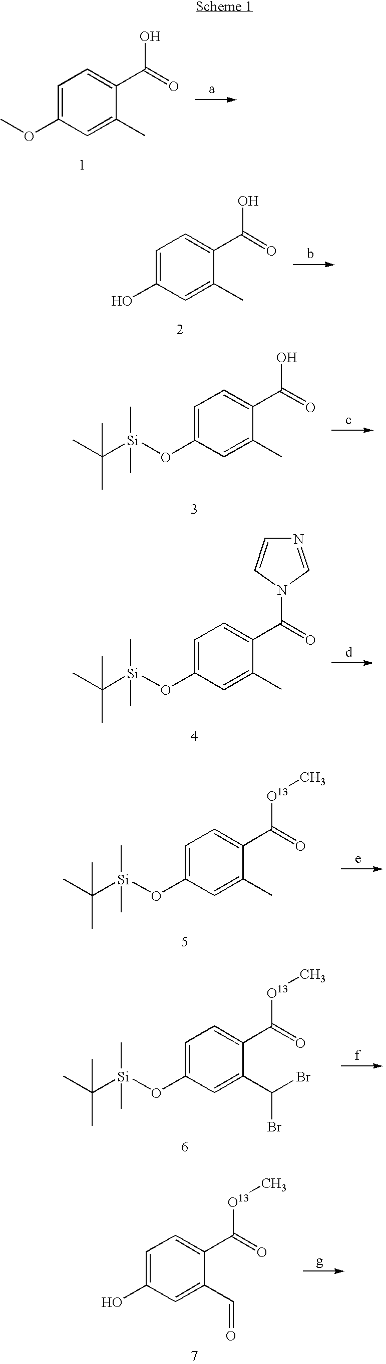

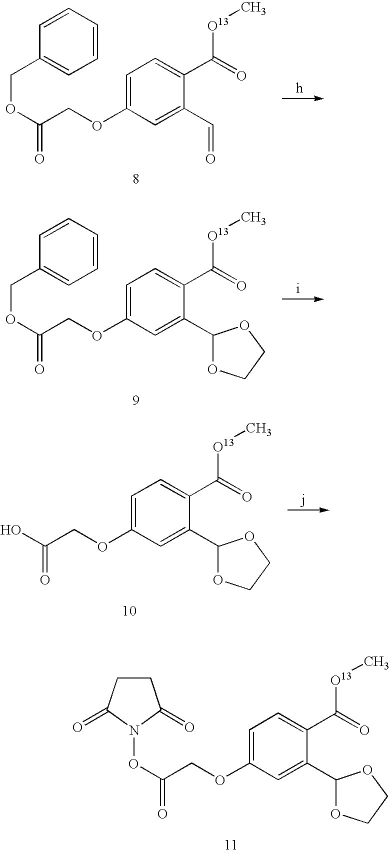

Reagents and conditions: a) BBr₃, −78° C., CH₂Cl₂; b) TBDMSCl, DMF, DIPEA, rt: c) CH₂Cl₂, carbonyl diimidazole, rt; d) CH₃OH, DCE, MW, 100° C., 2 min.; e) NBS, AIBN, CCl₄, reflux; f) AgNO₃, H₂O, i-PrOH, rt, then TBAF, DCM; g) Benzyl 2-bromoacetate, K₂CO₃, acetone, 0° C.; h) 2,6-Di-tert-butyl pyridine, 1,2-bis(trimethylsilyloxy)ethane, trimethylsilyl trifluoromethanesulfonate, 2-pyridylcarbinol, CH₂Cl₂, 0° C.; i) H₂, Pd/C, EtOAc; j) N-hydroxysuccinimide, PS-carbodiimide (Argonaut technologies), EtOAc.

4-Hydroxy-2-methylbenzoic acid (2). To a 250 mL flame dry 3-neck round bottom flask was added 4-methoxy-2-methyl benzoic acid (1) (5.0 g, 30.08 mmol) and CH₂Cl₂ (80 mL). The reaction was cooled to −78° C. and treated with neat BBr₃ (5.7 mL, 60.17 mmol) dropwise via an addition funnel. The reaction was stirred for 30 min at −78° C. The solution temperature was increased to −15° C. and stirred for 4 h (−15 to −10° C.). The cooling bath was removed. The reaction was stirred for 20 h at rt. The solution was cooled to 0° C. and quenched with ether (15 mL) and water (15 mL) (caution: water caused violent reaction; added water dropwise). The biphasic mixture was extracted with EtOAc (3×100 mL). The combined organic layers were dried over MgSO₄, filtered, and concentrated in vacuo. The crude product was purified by SiO₂ chromatography (300 g SiO₂, 70:30 hexanes-acetone, R_{f}=0.31) to afford the title compound. APCI MS (m/z): 151.12 (M−H); Calc'd. for C₈H₈O₃: 152.15. ¹H NMR (300 MHz, CHLOROFORM-d) δ ppm 2.55 (s, 3 H) 6.29-6.78 (m, 2 H) 7.90 (d, J=9.42 Hz, 1 H).

4-(tert-Butyldimethylsilyloxy)-2-methylbenzoic acid (3). To a stirred solution of 4-hydroxy-2-methylbenzoic acid (2) (4.2 g, 27.60 mmol) in DMF (20 mL) was added t-BDMSCl (10.2 g, 67.63 mmol) and stirred for 15 min. Dry i-Pr₂NEt (14.0 mL, 80.05 mmol) was added dropwise via an addition funnel and stirred at rt for 20 h. The reaction was quenched with 1M H₃PO₄ (7 mL) till the final pH was 3-4. The solution was extracted with hexanes (4×100 mL). The combined organic layers were dried over MgSO₄, filtered, and concentrated in vacuo. The crude product was purified by SiO₂ chromatography (300 g SiO₂, 90:9:1 hexanes-acetone-AcOH, R_{f}=0.28) to afford the title compound. APCI MS (m/z): 267.15 (M+H); Calc'd. for C₁₄H₂₂O₃Si: 266.13. ¹H NMR (300 MHz, CHLOROFORM-d) δ ppm 0.24 (s, 6 H) 0.99 (s, 9 H) 2.61 (s, 3 H) 6.61-6.78 (m, 2 H) 7.92-8.06 (m, 1 H).

(4-(tert-Butyldimethylsilyloxy)-2-methylphenyl)(1H-imidazol-1-yl)methanone (4). 4-(tert-Butyldimethylsilyloxy)-2-methylbenzoic acid (3) (5.8 g, 21.77 mmol) was dissolved in CH₂Cl₂ (50 mL) and treated with 1,1′-carbonyldiimidazole (4.2 g, 26.12 mmol) for 20 h under N₂ at rt. The solution was diluted with CH₂Cl₂ (50 mL). The organic layer was washed with water (2×50 mL), brine (2×30 mL), dried over MgSO₄, filtered, and concentrated in vacuo to afford the title compound (70:29:1 hexanes-acetone-NEt₃, R_{f}=0.14). APCI MS (m/z): 317.15 (M+H); Calc'd. for C₁₇H₂₄N₂O₃Si: 316.47. ¹H NMR (300 MHz, CHLOROFORM-d) δ ppm 0.25 (s, 6 H) 1.00 (s, 9 H) 2.39 (s, 3 H) 6.75 (dd, J=8.38, 2.17 Hz, 1 H) 6.81 (d, J=1.88 Hz, 1 H) 7.13 (s, 1 H) 7.33 (d, J=8.29 Hz, 1 H) 7.47 (s, 1 H) 7.92 (s, 1 H).

¹³C-Methyl 4-(tert-butyldimethylsilyloxy)-2-methylbenzoate (5). To a oven dry 20 mL Conical Smith Synthesizer tube was added (4-(tert-butyldimethylsilyloxy)-2-methylphenyl)(1H-imidazol-1-yl)methanone (4) (5.5 g, 17.38 mmol), DCE (10 mL), ¹³CH₃OH (Cambridge Isotope Laboratory, 2.2 mL, 52.13 mmol), and DBU (0.8 mL, 5.21 mmol). The tube was sealed and microwaved using a Smith Synthesizer for 2 min at 100° C. The reaction was concentrated in vacuo. The crude product was purified by SiO₂ chromatography (300 g SiO₂, 95:5 hexanes-acetone, R_{f}=0.65) to afford the title compound. APCI MS (m/z): 282.5 (M+H); Calc'd. for C₁₄¹³CH₂₄O₃Si: 281.15. ¹H NMR (300 MHz, CHLOROFORM-d) δ ppm 0.22 (s, 6 H) 0.98 (s, 9 H) 2.56 (s, 3 H) 3.85 (d, J=146.75 Hz, 3 H) 6.62-6.74 (m, 2 H) 7.86 (d, J=8.85 Hz, 1 H).

¹³C-Methyl 4-(tert-butyldimethylsilyloxy)-2-(dibromomethyl)benzoate (6). To a stirred solution of ¹³C-methyl 4-(tert-butyldimethylsilyloxy)-2-methylbenzoate (5) (4.0 g, 14.21 mmol) in CCl₄ (50 mL) was added N-bromosuccinimide (7.6 g, 42.64 mmol) and 2,2′-Azobisisobutyronitrile (2.3 g, 14.21 mmol). The reaction was heated to reflux (83° C.) under N₂ for 18 h. The reaction was cooled to rt and filtered. The solvent was removed from the filtrate in vacuo. The crude product was purified by SiO₂ chromatography (300 g SiO₂, 90:10 hexanes-acetone, R_{f}=0.78) to afford the title compound. APCI MS (m/z): 440.2 (M+H); Calc'd. for C₁₄¹³CH₂₂O₃Si: 439.22. ¹H NMR (300 MHz, CHLOROFORM-d) δ ppm 0.28 (s, 6 H) 1.01 (s, 9 H) 3.90 (d, J=147.31 Hz, 3 H) 6.80 (dd, J=8.67, 2.45 Hz, 1 H) 7.58 (d, J=2.45 Hz, 1 H) 7.83 (d, J=8.67 Hz, 1 H) 8.10 (s, 1 H).

¹³C-Methyl 2-formyl-4-hydroxybenzoate (7). To a stirred solution of ¹³C-methyl 4-(tert-butyldimethylsilyloxy)-2-(dibromomethyl)benzoate (6) (5.0 g, 11.38 mmol) in i-PrOH (60 mL) was added silver nitrate (3.86 g, 22.77 mmol) in water (6 ml). The resulting mixture was stirred under N₂ for 20 h. The reaction was filtered, and the filtrate was concentrated in vacuo. The residue was dissolved in CH₂Cl₂, dried over MgSO₄, filtered, and treated with 1M tetra-n-butylammonium fluoride in THF (6.6 ml, 22.77 mmol). After 3 h under N₂, the reaction was concentrated in vacuo. The crude product was purified by SiO₂ chromatography (120 g SiO₂, 80:20 hexanes-acetone, R_{f}=0.33) to afford the title compound. APCI MS (m/z): 182.2 (M+H); Calc'd. for C₈¹³CH₈O₄: 181.05. ¹H NMR (300 MHz, CHLOROFORM-d) δ ppm 3.95 (d, J=147.50 Hz, 3 H) 7.09 (dd, J=8.57, 2.73 Hz, 1 H) 7.40 (d, J=2.83 Hz, 1 H) 7.98 (d, J=8.48 Hz, 1 H) 10.69 (s, 1 H).

¹³C-Methyl 4-(2-(benzyloxy)-2-oxoethoxy)-2-formylbenzoate (8). ¹³C-Methyl 2-formyl-4-hydroxybenzoate (7) (1.55 g, 8.56 mmol) was dissolved in acetone (20 mL) and cooled to 0° C. Benzyl 2-bromoacetate (1.9 ml, 11.97 mmol) and potassium carbonate (1.4 g, 10.27 mmol) were added. The reaction was stirred under N₂ at 0° C. for 18 h. The reaction was quenched with water (5 mL) and the solvent was removed in vacuo. The residue was partitioned between EtOAc (100 mL) and water (40 mL). The layers were separated, and the organic layer was washed with water (2×20 mL), brine (1×20 mL), dried over MgSO₄, filtered, and concentrated in vacuo. The crude product was purified by SiO₂ chromatography (120 g SiO₂, 85:15 hexanes-acetone, R_{f}=0.35) to afford the title compound. APCI MS (m/z): 330.1(M+H); Calc'd. for C₁₇¹³CH₁₆O₆: 329.09. ¹H NMR (300 MHz, CHLOROFORM-d) δ ppm 3.95 (d, J=147.69 Hz, 3 H) 4.77 (s, 2 H) 5.25 (s, 2 H) 7.15 (dd, J=8.67, 2.64 Hz, 1 H) 7.32-7.43 (m, 6 H) 7.98 (d, J=8.67 Hz, 1 H) 10.68 (s, 1 H).

¹³C-Methyl 4-(2-(benzyloxy)-2-oxoethoxy)-2-(1,3-dioxolan-2-yl)benzoate (9). ¹³C-Methyl 4-(2-(benzyloxy)-2-oxoethoxy)-2-formylbenzoate (8) (2.17 g, 6.6 mmol) was dissolved in CH₂Cl₂ (20 mL) and cooled to 0° C. 2,6-di-tert-butylpyridine (0.150 ml, 0.66 mmol), 1,2-bis(trimethylsilyloxy)ethane (2.4 ml, 9.88 mmol), and trimethylsilyl trifluoromethanesulfonate (0.180 ml, 0.98 mmol) was added. The reaction was stirred at 0° C. under N₂ for 18 h. The solution was quenched with 2-pyridylcarbinol (0.127 ml, 1.32 mmol). The solvent was removed in vacuo. The crude product was purified by SiO₂ chromatography (120 g SiO₂, 80:20 hexanes-acetone, R_{f}=0.22) to afford the title compound. APCI MS (m/z): 374.1 (M+H); Calc'd. for C₁₉¹³CH₂₀O₇: 373.12. ¹H NMR (300 MHz, CHLOROFORM-d) δ ppm 3.88 (d, J=147.12 Hz, 3 H) 3.97-4.06 (m, J=2.26 Hz, 4 H) 4.73 (s, 2 H) 5.24 (s, 2 H) 6.65 (s, 1 H) 6.88 (dd, J=8.67, 2.83 Hz, 1 H) 7.30 (d, J=2.83 Hz, 1 H) 7.35 (s, 5 H) 7.91 (d, J=8.67 Hz, 1 H).

2-(3-(1,3-Dioxolan-2-yl)-4-(¹³C-methoxycarbonyl)phenoxy)acetic acid (10). To a stirred solution of ¹³C-methyl 4-(2-(benzyloxy)-2-oxoethoxy)-2-(1,3-dioxolan-2-yl)benzoate (9) (1.72 g, 4.6 mmol) in EtoAc (25 ml) was added palladium 10% on carbon (170 mg). The solution was degassed with three cycles of evacuation/nitrogen refill. After last evacuation, H₂ from a balloon was used to backfilled the final evacuation. The reaction was stirred at rt under H₂ for 3 h. The solution was filtered through a pad of celite. The solvent was removed from the filtrate in vacuo to afford the title compound (60:40 hexanes-acetone, R_{f}=0.11). APCI MS (m/z): 284.3 (M+H); Calc'd. for C₁₂¹³CH₁₄O₇: 283.08. ¹H NMR (300 MHz, CHLOROFORM-d) δ ppm 3.89 (d, J=147.12 Hz, 3 H) 4.06 (s, 4 H) 4.75 (s, 2 H) 6.65 (s, 1 H) 6.92 (dd, J=8.76, 2.73 Hz, 1 H) 7.34 (d, J=2.83 Hz, 1 H) 7.94 (d, J=8.67 Hz, 1 H).

¹³ C-Methyl 4-(N-(succinimideoxy)-2-oxoethoxy)-2-(1,3-dioxolan-2-yl)benzoate (11). To a solution of 2-(3-(1,3-dioxolan-2-yl)-4-(¹³C-methoxycarbonyl)phenoxy)acetic acid (10)

(1.21 g, 4.27 mmol) in EtOAc (20 ml) was added 1-hydroxypyrrolidine-2,5-dione (0.74 g, 6.41 mmol) and PS-carbodiimide (Argonunt Technology, 1.29 mmol/g) (4.6 g, 5.98 mmol). The reaction was sealed and stirred at rt for 20 h. The solution was filtered using a medium porosity sintered glass funnel. The resin was agitated with EtOAc (20 mL) by bubbling N₂ through the sintered glass for 10 min. The EtOAc was filtered and combined with the first filtrate. The resin was washed a second time using the same protocol. The combined filtrates were concentrated in vacuo. The crude product was purified by SiO₂ chromatography (120 g SiO₂, 70:29:1 hexanes-acetone-AcOH, R_{f}=0.14) to afford the title compound. APCI MS (m/z): 381.2 (M+H); Calc'd. for C₁₆¹³CH₁₇NO₉: 380.09. ¹H NMR (300 MHz, CHLOROFORM-d) δ ppm 2.87 (s, 4 H) 3.89 (d, J=147.12 Hz, 3 H) 4.02-4.10 (m, J=1.70 Hz, 4 H) 5.04 (s, 2 H) 6.67 (s, 1 H) 6.95 (dd, J=8.67, 2.83 Hz, 1 H) 7.35 (d, J=2.83 Hz, 1 H) 7.95 (d, J=8.67 Hz, 1 H).

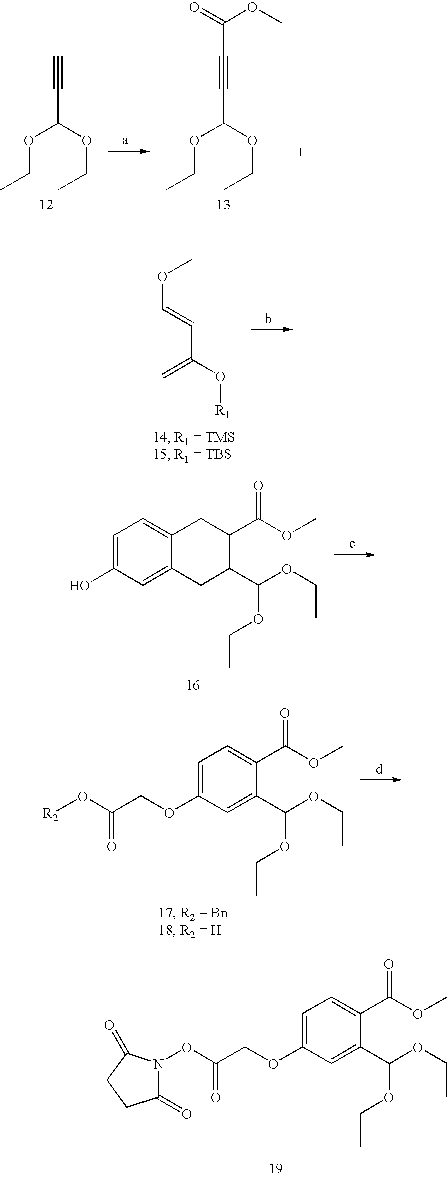

Reagents and conditions: a) n-BuLi, then methyl chloro formate; b) Toluene, 170° C.; c) Benzylbromoacetate, K₂CO₃, acetone; d) H₂, Pd/C, EtOAc. Methyl 4,4-diethoxybut-2-ynoate (13). A solution of diethoxypropyne (Aldrich, 10.93 g, 85.3 mmol) in diethyleneglycol-dimethylether (100 mL) was cooled to −30° C. under N₂. n-Butyllithium (81.0 mmol) was added dropwise over 5 min. The reaction was incubated for 6 h. The formed anion was cannulated to a solution of methyl chloroformate (6.5 mL, 84.1 mmol) in 50 mL diethyleneglycol-dimethylether with overhead stirring in a dry ice/acetone bath under N₂. The reaction warmed to room temperature overnight. The solids were removed by filtration through a pad of alumina (100 g of basic alumina, rinsed with 200 mL ether). The solution was concentrated completely by rotary evaporation (bath temp=35° C.). The solids were removed by filtration through a pad of alumina (rinsed with 500 mL ether, 100 g basic alumina). The solution was concentrated completely by rotary evaporation (bath temp=35° C.). The product was purified by distillation (fraction boiled at 57-60° C. at 1 mm Hg) to afford the title compound. ¹H NMR (300 MHz, CHLOROFORM-d) δ ppm: 1.24 (d, J=14.32 Hz, 6 H) 3.56-3.68 (m, 2 H) 3.68-3.83 (m, 2 H) 3.79 (s, 3 H) 5.36 (s, 6 H). GC-MS: Method 1: 4.22 min (EI MS (m/z)=141 (M-OEt); calc'd for C₇H₉O₃⁺: 141).

Methyl 2-(diethoxymethyl)4-hydroxybenzoate (16). To a oven-dry 5 mL Conical Smith Synthesizer tube was added methyl 4,4-diethoxybut-2-ynoate (0.25 g, 1.3 mmol) (13), (E)-(4-methoxybuta-1,3-dien-2-yloxy)tri-methylsilane (0.52 ml, 2.7 mmol) (12), 4-(3,5-di-tert-butyl-4-hydroxy-benzyl)-2,6-di-tert-butylphenol (0.11 g, 0.27 mmol), and toluene (4 ml). The tube was sealed and heated for at 170° C. for 20 h. The reaction was cooled to rt, transferred to a round bottom flask, and treated with 1M tetra-n-butylammonium fluoride in THF (0.78 ml, 2.7 mmol). The solution was sealed and stirred at rt for 3 h. The solvent was removed in vacuo. The crude product was purified by SiO₂ chromatography (40 g SiO₂, 80:20 hexanes-acetone, R_{f}=0.42) to afford the title compound. APCI MS (m/z): 255.2 (M+H); Calc'd. for C₁₃H₁₈O₅: 254.12. ¹H NMR (300 MHz, CHLOROFORM-d) δ ppm 1.23 (t, J=7.06 Hz, 6 H) 3.53-3.66 (m, 2 H) 3.66-3.78 (m, 2 H) 3.87 (s, 3H) 5.59 (s, 1 H) 6.26 (s, 1 H) 6.80 (dd, J=8.57, 2.73 Hz, 1 H) 7.30 (d, J=2.64 Hz, 1 H) 7.82 (d, J=8.67 Hz, 1 H).

Methyl 4-(2-(benzyloxy)-2-oxoethoxy)-2-(diethoxymethyl)benzoate (17). To a 0° C., stirred solution of methyl 2-(diethoxymethyl)-4-hydroxybenzoate (0.5 g, 2 mmol) (16) in acetone (15 mL) was added benzyl 2-bromoacetate (0.4 ml, 3 mmol) and potassium carbonate (0.3 g, 2 mmol). The solution was stirred under N₂ at 0° C. for 20 h. The solution was quenched with water (5 mL) and the solvent was concentrated in vacuo. The residue was partitioned between EtOAc (75 mL) and water (30 mL). The layers were separated, and the organic layer was washed with water (2×20 mL), brine (1×20 mL), dried over MgSO₄, filtered, and concentrated in vacuo. The crude product was purified by SiO₂ chromatography (40 g SiO₂, 85:15 hexanes-acetone, R_{f}=0.35) to afford the title compound. APCI MS (m/z): 255.2 (M-EtOH). Calc'd. for C₂₂H₂₆O₇: 402.17. ¹H NMR (300 MHz, CHLOROFORM-d) δ ppm 1.21 (t, J=6.97 Hz, 6 H) 3.53-3.60 (m, 2 H) 3.62-3.74 (m, 2 H) 3.87 (s, 3 H) 4.73 (s, 2 H) 5.24 (s, 2 H) 6.22 (s, 1 H) 6.86 (dd, J=8.67, 2.64 Hz, 1 H) 7.35 (s, 6 H) 7.83 (d, J=8.67 Hz, 1 H).

2-(3-(Diethoxymethyl)-4-(methoxycarbonyl)phenoxy)acetic acid (18). To a stirred solution of Methyl 4-(2-(benzyloxy)-2-oxoethoxy)-2-(diethoxymethyl)-benzoate (0.65 g, 1.6 mmol) (17) in EtOAc (15 ml) was added palladium (0.052 g, 0.48 mmol). The solution was degassed with three cycles of evacuation/nitrogen refill. After last evacuation, H₂ from a balloon was used to backfilled the final evacuation. The reaction was stirred at rt under H₂ for 3 h. The solution was filtered through a pad of celite. The solvent was removed in vacuo to afford the title compound (70:29:1 hexanes-acetone-AcOH, R_{f}=0.21). APCI MS (m/z): 311.1 (M−H). Calc'd. for C₁₅H₁₉O₇: 311.1. ¹H NMR (300 MHz, CHLOROFORM-d) δ ppm 1.22 (t, J=6.97 Hz, 6 H) 3.51-3.64 (m, 2 H) 3.64-3.78 (m, 2 H) 3.88 (s, 3 H) 4.74 (s, 2 H) 6.24 (s, I H) 6.90 (dd, J=8.67, 2.83 Hz, 1 H) 7.36 (d, J=2.64 Hz, 1 H) 7.86 (d, J=8.67 Hz, 1 H).

Methyl 4-(N-(succinimideoxy)-2-oxoethoxy)-2-(1,3-dioxolan-2-yl)benzoate (19). To a stirred solution of 2-(3-(diethoxymethyl)-4-(methoxycarbonyl)phenoxy)-acetic acid (450 mg, 1.44 mmol) (18) in EtOAc (15 ml) was added N-hydroxysuccinimide (248 mg, 2.16 mmol) and PS-carbodiimide (Argonaunt Technology, 1.29 mmol/g) (1.5 g, 2.02 mmol). The reaction was sealed and stirred at rt for 20 h. The solution was filtered using a medium porosity sintered glass funnel. The resin was agitated with EtOAc (20 mL) by bubbling N₂ through the sintered glass for 10 min. The EtOAc was filtered and combined with the first filtrate. The resin was washed a second time using the same protocol. The combined filtrates were concentrated in vacuo. The crude product was purified by SiO₂ chromatography (40 g SiO₂, 80:19:1 hexanes-acetone-AcOH, R_{f}=0.38) to afford the title compound. APCI MS (m/z): 364.23 (M+H-OEt). Calc'd. for C₁₇H₁₈NO₈⁺: 364.1. ¹H NMR (300 MHz, CHLOROFORM-d) δ ppm 1.23 (t, J=7.16 Hz, 6 H) 2.87 (s, 4 H) 3.54-3.76 (m, 4 H) 3.87 (s, 3 H) 5.03 (s, 2 H) 6.23 (s, 1 H) 6.91 (dd, J=8.67, 2.83 Hz, 1 H) 7.40 (d, J=2.64 Hz, 1 H) 7.86 (d, J=8.67 Hz, 1 H).

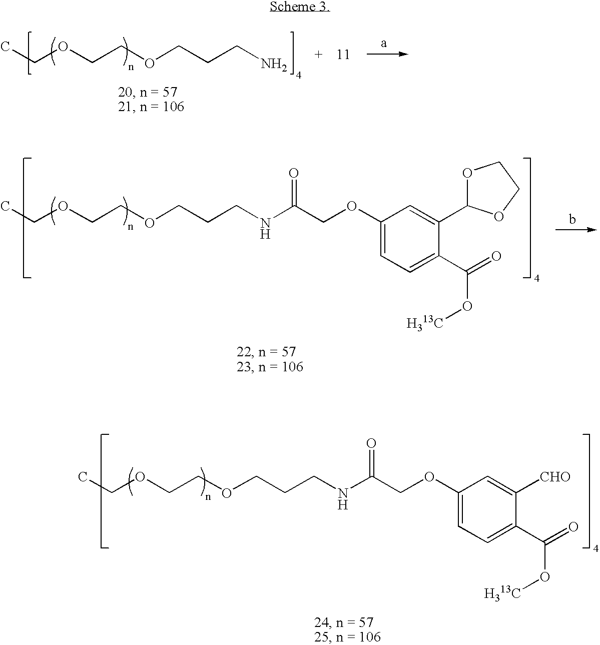

Reagents and Conditions: a) Acetonitrile, 25° C.; b) DCl, D₂O.

Tetrakis-[(ω-(4-aza-5-oxo-7-oxa-7-((3-(2,4-dioxacyclopentyl)-4-(¹³C-methoxy)-carbonyl)benzene)heptane)-2.5 kD polyoxyethylene]methane 22. PTE-100 PA (NOF corp, 547 mg, ˜52 μmol) was dissolved in 2.5 mL dry acetonitrile and treated with succinate 11 (100 mg, 260 μmol, 5 eq). The reaction was heated to 40° C. for 7 h. The reaction was cooled to rt and treated with 10 mM NH₄ formate (10 mL). The solution was loaded onto column 1 and eluted with solvent system 1/gradient table 1 as defined in the Preparative Reverse Phase HPLC section of the general experimental. A band eluting from 27.4-28.8 minutes was isolated, and concentrated in vacuo to remove acetonitrile. The aqueous solution was filtered through a 0.22 μm centrifugal filter (National Scientific, PN 66064-466) at 2560 g and the filtrate lyophilized. The solid was dissolved in 5 mL D₂O and lyophilized to afford the product. ¹H NMR (400 MHz, DEUTERIUM OXIDE) δ ppm 1.78 (p, J=6.65 Hz, 2 H) 3.35 (t, J=6.46 Hz, 2 H) 3.45 (t, J=6.26 Hz, 2 H) 3.48-3.52 (m, 2 H) 3.70 (s, (CH₂CH₂O)ₙ) 3.90 (d, J=149.07 Hz, 3 H) 4.09-4.16 (m, 4 H) 4.71 (s, 2 H) 6.51 (s, 1 H) 7.12 (dd, J=8.61, 2.74 Hz, 1 H) 7.31 (d, J=2.74 Hz, 1 H) 7.97 (d, J=8.61 Hz, 1 H) 8.40 (s, 1 H). ¹³C NMR (101 MHz, DEUTERIUM OXIDE) δ ppm 55.08 (s, 4C), 72.00 (s, 5C).

Tetrakis-[ω-(4-aza-5-oxo-7-oxa-7-((3-(2,4-dioxacyclopentyl) -4-(¹³C-methoxy)-carbonyl)benzene)heptane)-5.0 kD polyoxyethylene]methane 23. PTE-200 PA (NOF corp, 1.55 g, ˜64 lmol; certificate of analysis: 83% tetrafunctionalized), succinate 11 (147 mg, 386 μmol) and 5 mL acetonitrile were heated to 40° C. for 4 h. The acetonitrile was removed and 5 mL 0.1% acetic acid was added. The solution was heated to 35° C. to aid dissolution. The solution was loaded onto column 1 (column jacket and solvents were heated to 35° C.), and eluted with solvent system 2/gradient table 1 as defined in the Preparative Reverse Phase HPLC section of the general experimental. A band eluting from 22.8 to 26 min was concentrated in vacuo and dried at 35° C. under reduced pressure (1 mm Hg). The residue was dissolved in 10 mL D₂O and lyophilized to afford the product. The solid was determined to be a 6:1 mixture of 23 and 25 by ¹H NMR; ¹H NMR provided for 23. ¹H NMR (400 MHz, DEUTERIUM OXIDE) δ ppm 1.78 (p, J=6.06 Hz, 2 H) 3.35 (t, J=6.65 Hz, 2 H) 3.45 (t, J=6.26 Hz, 2 H) 3.50 (s, 2 H) 3.70 (s, (CH₂CH₂O)ₙ) 3.90 (d, J=147.12 Hz, 3 H) 4.09-4.16 (m, 4 H) 4.71 (s, 2 H) 6.51 (s, 1 H) 7.12 (dd, J=8.61, 2.74 Hz, 1 H) 7.31 (d, J=2.74 Hz, 1 H) 7.98 (d, J=9.00 Hz, 1 H). ¹³C NMR (101 MHz, DEUTERIUM OXIDE) δ ppm 55.08 (s, 9.98 C) 72.00 (s, 2.84 C).

Tetrakis-[ω-(4-aza-5-oxo-7-oxa-7-((3-formyl-4-(¹³C-methoxy)carbonyl)benzene)-heptane)-2.5 kD polyoxyethylene]methane 24. PEG reagent 22 (439 mg, 38.3 μmol) was dissolved in 5 mL D₂O, cooled to 0° C. and degassed by 4 cycles of evacuation/nitrogen refill. A 85 mM solution of DCl in D₂O (360 μL, 0.2 eq. per acetal) was added. The cooling bath was removed and the reaction was stirred at rt for 24 h. After 24 h, an additional portion of DCl was added (360 μL). The reaction was stirred for 63 h. The aqueous solution was lyophilized and dissolved in 2 mL D₂O. The solution was filtered through a 0.1 μm centrifugal filter (Micron Bioseparations, PN UFC40WOO) and lyophilized to afford the product. ¹H NMR (400 MHz, DEUTERIUM OXIDE) δ ppm 1.80 (p, J=6.10 Hz, 2 H) 3.36 (t, J=6.46 Hz, 2 H) 3.45-3.54 (m, 4 H) 3.70 (s, (CH₂CH₂O)ₙ) 3.96 (d, J=148.6 Hz, 3 H) 4.72 (s, 2 H) 7.32 (d, J=9.00 Hz, 1 H) 7.38 (s, 1 H) 8.01 (d, J=8.61 Hz, 1 H) 8.26 (s, 1 H) 10.42 (s, 1 H).

Tetrakis-[ω-(4-aza-5-oxo-7-oxa-7-((3-formyl-4-(¹³C-methoxy)carbonyl)benzene)-heptane)-5.0 kD polyoxyethylene]methane 25. PEG reagent 23 (840 mg, 39 μmol) was dissolved in 10 mL H₂O, cooled to 0° C. and treated with 85 mM DCl in D₂O (183 μL, 15.6 μmol, 0.1 eq per acetal). After 4.5 d, the reaction was lyophilized, dissolved in 10 mL D₂O and treated with 85 mM DCl in D₂O (183 μL, 15.6 μmol) for 1 d at room temperature. The solution was lyophilized to afford the product. ¹H NMR (400 MHz, DEUTERIUM OXIDE) δ ppm 1.79 (p, J=6.31 Hz, 2 H) 3.36 (t, J=6.65 Hz, 2 H) 3.43-3.55 (m, 4 H) 3.70 (s, (CH₂CH₂O)ₙ) 3.96 (d, J=148.68 Hz, 3 H) 4.74 (s, 2 H) 7.34 (dd, J=8.61, 2.74 Hz, 1 H) 7.42 (d, J=2.74 Hz, 1 H) 8.03 (d, J=8.61 Hz, 1 H) 10.44 (s, 1 H).

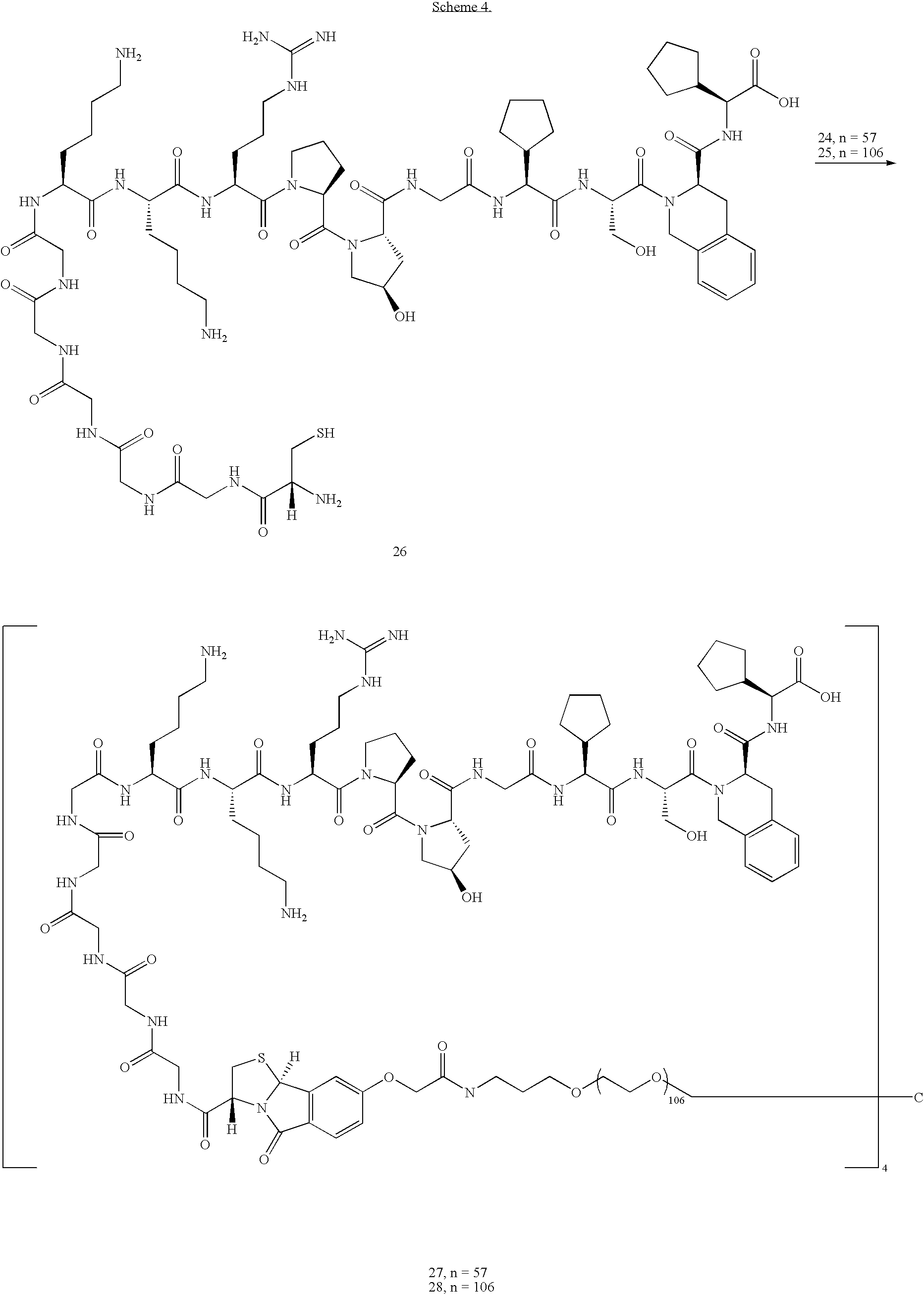

Reagents and Conditions: 600 mM LiCl, pH 2.5-6 ascorbate buffer. Tetrakis-[ω-(4-aza-5-oxo-7-oxa-7-(((3′R,9′bS)-3′-(carbonyl(HN-GGGGGKKRP-(Hyp)G(Cpg)S(D-Tic)(Cpg)-OH))-2′,3′-dihydrothiazolo[2′,3′-a]isoindol-5′(9′bH)-one-8′-yl))heptane)-2.5 kD polyoxyethylene]methane 27. Peptide 26 (1.12 g, PPL laboratories) was dissolved in 1.8 mL D₂O, treated with 0.25 mL 0.50 M sodium ascorbate/4.8 M LiCl in D₂O and cooled to 0° C. The solution was degassed with three cycles of evacuation/nitrogen refill. The pH was adjusted under anitrogen with 1 M LiOH to 6.1, and degassed with three cycles of evacuation/nitrogen refill. The peptide concentration was determined to be 114.4 mM by HPLC with Chemiluminescence nitrogen detection (CLND) calibrated against caffeine as described in the general experimental section. PEG reagent 24 (400 mg, 35.4 μmol) was dissolved in 2.5 mL D₂O and successively treated with 0.25 mL 0.50 M sodium ascorbate/4.8 M LiCl in D₂O and 0.25 mL 0.55 ascorbic acid/4.86 M LiCl in D₂O. Peptide 26 (1.4 mL, 159.4 μmol) was then added. The pD of the solution was determined to be 5.1. The reaction stirred for 3d at rt under nitrogen. The solution was loaded onto column 1, and eluted with solvent system 2/gradient table 1 as defined in the Preparative Reverse Phase HPLC section of the general experimental. A band eluted at 12.2-15.4 minutes was concentrated in vacuo to remove acetonitrile (bath temperature=35° C.) and lyophilized to dryness. The residue was further purified using cation exchange column 1, eluted with solvent system 1/gradient table 1 as described Preparative Ion exchange section of the general experimental. A band eluted from 41.2 to 58.2 min was concentrated to dryness by rotary evaporation (bath temp=35° C.). The residue was dissolved in 10 mL water, charged to a 3500 MWCO dialysis membrane (Pierce, PN 65035) and dialysed against deionized water (3×500 mL, 1-2 h each cycle). The dialysed solution was lyophilized to afford the product. CLND: 29.3%; theory: 36.3%. Selected NMR resonances diagnostic for chemistry used for attachment: ¹H NMR (400 MHz, DEUTERIUM OXIDE) δ ppm 4.85 (dd, J=14.87 Hz, 1 H) 4.98 (t, J=7.04 Hz, 1 H) 5.01-5.07 (m, 1 H) 5.17 (t, J=5.48 Hz, 1 H) 5.30-5.40 (m, 1 H) 6.19 (s, 1 H) 7.13-7.36 (m, 1 H) 5.17 J=8.61 Hz, 1 H).

Tetrakis-[ω-(4-aza-5-oxo-7-oxa-7-(((3′R,9′bS)-3′-(carbonyl(HN-GGGGGKKRP-(Hyp)G(Cpg)S(D-Tic)(Cpg)-OH))-2′,3′-dihydrothiazolo[2′,3′-a]isoindol-5′(9′bH)-one-8′-yl))heptane)-5.0 kD polyoxyethylene]methane 28. PEG reagent 28 (99.4 mg, 4.66 μmol) was dissolved in 2 mL D₂O and treated with 0.5 mL 0.50 M sodium ascorbate/4.8 M LiCl in D₂O. The pD was determined to be 4.3. To this solution was added peptide 26 (72% peptide content, 47.4 mg, 21.7 μmol).

The reaction was stirred at room temperature for 18 h under a nitrogen atmosphere, and then heated to 45° C. for 2 h. The solution was loaded onto column 2, and eluted with solvent system 2/gradient table 2 as defined in the Preparative Reverse Phase HPLC section of the general experimental. A band that eluted from 10.5-12 minutes was collected and concentrated to 2 mL in vacuo (bath temperature=34° C.). The solution was loaded onto cation exchange column 2, and eluted with solvent system 1/gradient table 2 as defined in the Preparative Cation Exchange LC section of the general experimental. A band that eluted from 20-24 minutes was concentrated in vacuo (bath temp=35° C.) and dialysed with a 10K MWCO Slide-a-lyzer (Pierce, PN=66810) against 500 mL deionized water. The water was replaced with fresh 500 mL portions at 2-, 10- and 2 h. The dialysed solution was filtered through a 0.22 μm centrifugal filter (National Scientific, PN 66064-466) at 2560 g and the filtrate lyophilized to afford the title compound. CLND: 22.4% peptide content; theory: 23.2%. This sample was used for detailed structural characterization.

Detailed structural analysis for conjugate 28:

NMR Experiments.

The NMR experiments were performed in 3 mm tube using 5 mm inverse-detection cryoprobe on a Bruker drx-600 spectrometer.

### Chemical Shift Assignments

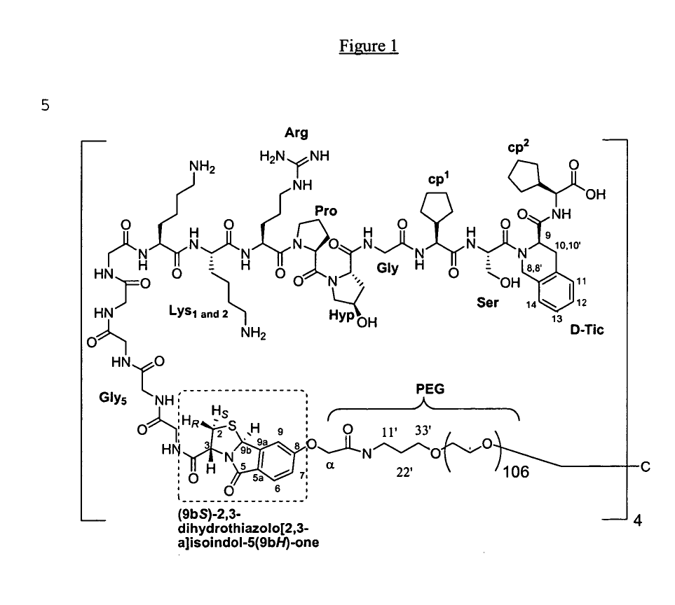

The proton chemical shifts for 28 (FIG. 1) were assigned based on the 2D TOCSY (100 ms DIPSI-2 mixing time) and 2D ¹³C-¹H HMBC (60 ms evolution of ⁿJ_{CH}, n=1-4). Only resonances from the major rotamer (trans) are listed in Table 2. Minor rotamer(s) originate from the hindered C-terminal and proline(s) amide bond rotations.

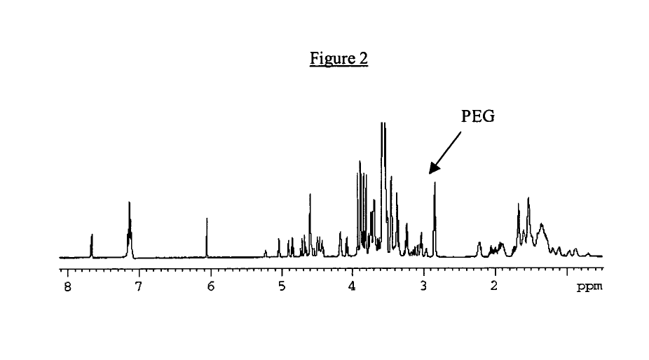

TABLE 1
Proton chemical shift assignments for FIG. 2, PEG singlet
set to δ 3.55 ppm.. The residue order is PEG→cp²,
as depicted in Scheme 1.

| Region | Proton(s) | Chemical shift(s) [ppm] |
|---|---|---|
| PEG | α | 4.59 |
| | 11′ | 3.36 |
| | 22′ | 1.66 |
| | 33′ | 3.22 |
| (9bS)-2,3- | 9b | 6.06 |
| dihydrothiazolo[2,3- | 2R | 3.73 |
| a]isoindol-5(9bH)- | 2S | 3.61 |
| one | 3 | 4.85 |
| | 6 | 7.67 |
| | 7 | 7.11 |
| | 9 | 7.14 |
| (Gly)₅ | αα′ | 3.89, 3.81 |
| Gly | αα′ | 4.53, 4.44 |
| Lys_{1 and 2} | α | 4.15-4.17 |
| | βγδ | 1.3-1.67 |
| | ε | 2.84 |
| Arg | α | 4.46 |
| | β | 1.64 |
| | γ | 1.53 |
| | δ | 3.02 |
| Pro | α | 4.58 |
| | ββ′ | 2.22, 1.73 |
| | γγ′ | 1.87-1.9 |
| | δδ′ | 3.73, 3.39 |
| Hyp | α | 4.49 |
| | ββ′ | 2.20, 1.93 |
| | γ | 4.41 |
| | δδ′ | 3.72, 3.67 |
| Cpg | α | 4.07 |
| | β | 2.05 |
| | γγ′, δδ′ | 1.60, 1.49, 1.36, 1.18, 1.10 |
| Ser | α | 5.03 |
| | ββ′ | 3.71, 3.67 |
| D-TIc | 88′ | 4.72, 4.66 |
| | 9 | 4.90 |
| | 10, 10′ | 3.12, 3.06 |
| | 11, 12, 12, 14 | 7.09-7.16 |
| Cpg | α | 3.82 |
| | β | 1.98 |
| | γγ′δδ′ | 1.41-1.35, 0.94, 0.86 |

Correlation of PEG Resonances to Peptide.

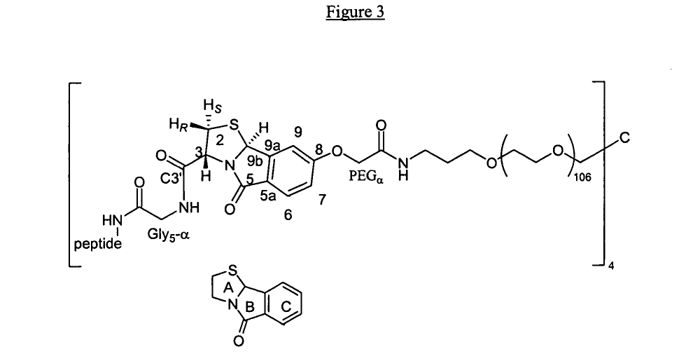

Three bond, ¹H—¹³C correlation spectroscopy was used to establish the site of PEGylation. The phenoxyacetamide methylene (PEGₐ, FIG. 3) was used as a starting point (4.58 ppm, 600 MHz, table 3). The observed correlation path was PEGₐ (4.59 ppm) to C₈ (162.2 ppm) to H₆ (7.67 ppm) to C₅ (173.2 ppm) to H₃ (4.85 ppm) to C_{3′} (172.7 ppm) to Glys_{5-a.} (3.89 ppm). The formation of the central B ring was supported by the observed correlation between C₅ (173.2 ppm) and H_{9b} (6.06 ppm). Similarly, the formation of the A ring was supported by a correlation sequence of H₃ (4.85 ppm) to C_{9b} (67.2 ppm) to H_{2R} (3.73 ppm). The H_{2R} signal showed correlation to C_{3′}, which supports the A ring proximity to gly₅ of the peptide.

TABLE 2
2D NOE derived and averaged MD interproton distances for the (9bS)-
2,3-dihydrothiazolo[2,3-a]isoindol-5(9bH)-one ring (FIG. 3).

| | | Predicted <r> for | |
|---|---|---|---|
| Proton-proton | Measured <r> | the trans-isomer | Predicted <r> for |
| pair | (Å, 2D NOESY) | (Å) | the cis-isomer (Å) |
| H_{9b}—H₃ | 4.4 | 4.1 | 3.5 |
| H₃—H_{2S} | 3.2 | 3.0 | 2.4 |
| H₃—H_{2R} | 2.7 | 2.6 | 3.1 |
| H_{9b}—H_{2S} | 3.2 | 3.4 | 2.7 |
| H_{9b}—H_{2R} | 3.9 | 4.2 | 4.2 |

The predicted dihedral angle formed by the atoms H₄—C₄—N—C₁ and H₁—C₁—N—C₄ for both the cis- and trans-diastereomers is given in table 3. From these angles, the 3-bond coupling constants were derived for H₃—C_{9b} and H_{9b}—C₃. The observed couplings of 8 and 0 Hz for H₃—C_{9b} and H_{9b}—C₃, respectively, agrees with the proposed trans-diastereomer as depicted in FIG. 1. Additionally, correlation was observed in the HMBC 2D experiment.

TABLE 3
Predicted dihedral angles and 3-bond C—H coupling for 28.
Atom labels are defined by FIG. 1.

| | | Predicted for | |
|---|---|---|---|
| | Predicted for Cis | Trans | experiment |
| θ(H₃C₃N₄C_{9b}) | −68 | 152 | — |
| ³J(H₃, C_{9b}) | 0 Hz | 8 Hz | 8 Hz |
| θ(H_{9b}C_{9b}N₄C₃) | 86 | 87 | — |
| ³J(H_{9b}, C₃) | 0 Hz | 0 | 0 Hz |
| H₃—C_{9b} | No cross-peak | Cross peak | Cross peak |
| H_{9b}—C₃ | No cross-peak | No cross-peak | No cross-peak |

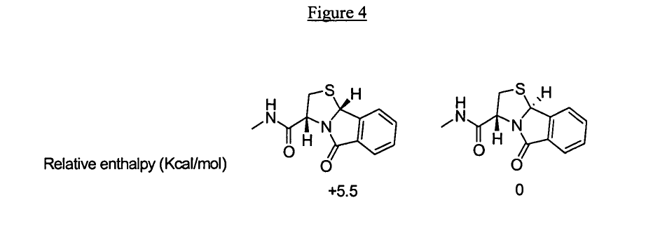

Molecular mechanics calculation suggest the trans-diastereomer has an enthalpy that is 5.5 kcal/mol lower that the cis-diastereoisomer (FIG. 4). The measured distance from carbonyl₅ and the amide NH of Gly₅ was determined to be 2.1 Å. This supports the presence of an intramolecular hydrogen bond.

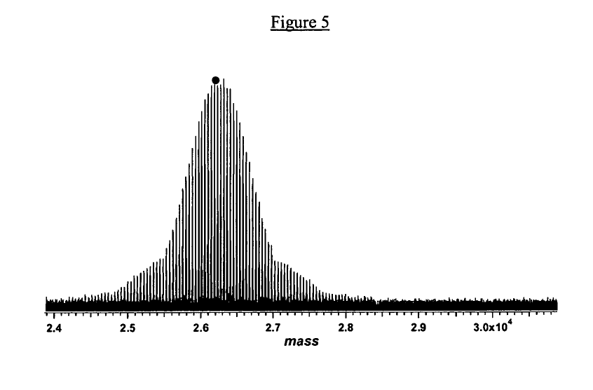

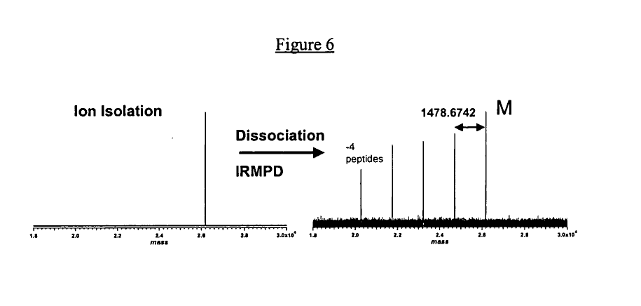

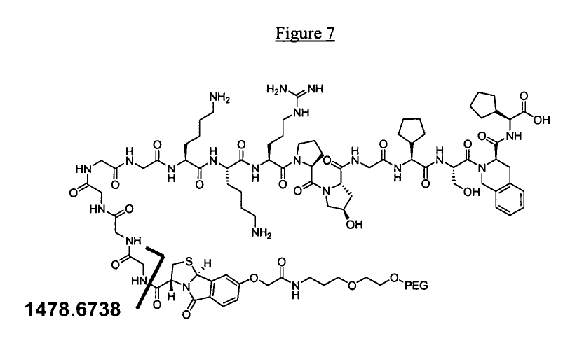

Conjugate 28 were analyzed with a Bruker Q-FTMS system, equipped with a 7-T superconducting magnet. Individual ions were isolated using the front end quadrupole. Ions were trapped in the FTMS cell employing “gas-assisted dynamic trapping.” Solutions were electrosprayed from a 4:1 MeOH—H2O solution at a flow rate of 0.5 uL/min. For IRMPD dissociation experiments a Synrad CO2 laser was turned on for 200 ms at a laser power of 15%. Ions were detected with direct mode detection at an acquisition bandwidth of 900 kHz and 512 K data points were collected. The time domain data were apodized and zero-filled once prior to performing a magnitude mode Fourier transform. The instrument was externally calibrated using the Agilent tuning mix. In this experiment (FIG. 5), the full deconvoluted spectra representing the heterogeneity of the polymer was obtained. One discrete isomer, with 420 repeating —(CH₂CH₂O)— units, was trapped in the FT-MS cell and irradiated with a IR laser (FIG. 6). This caused the ion to dissociate to give four daughter fragments, each separated by 1478.6742 amu. These data are consistent with the presence of four peptides per polymer and that the dissociation occurred between glycine₅ and the newly formed tricyclic ring system (FIG. 7).

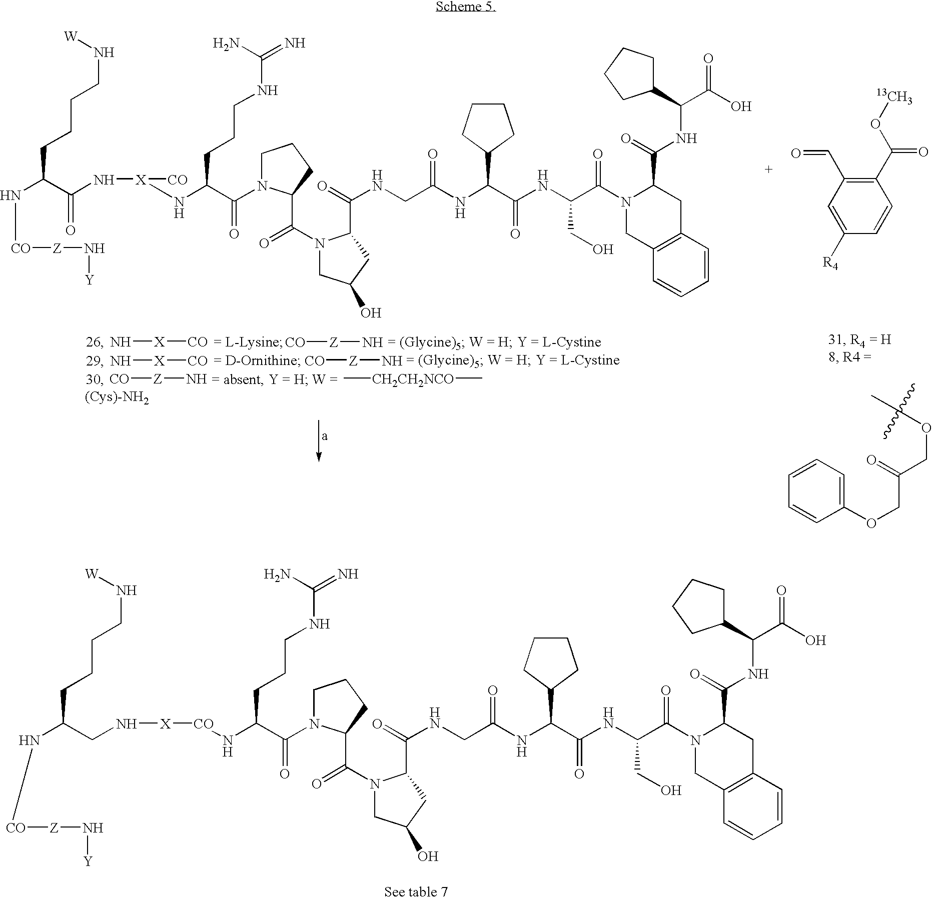

Reagents and conditions: a) methanol-water, 100 mM L-ascorbic acid, 20 mM sodium-L-ascorbate.

TABLE 7
Native ligation using 2-formyl esters.

| | | | | | Found APCI |
|---|---|---|---|---|---|
| | | | | | MS (m/z); |
| | | | | | calc'd |
| 32 | L-Lys | H | (Gly)₅ | 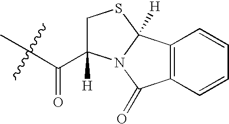 | 848.8971 (M + 2, z = 2); calc'd for C₇₈H₁₁₅N₂₁O₂₀S (z = 2): 848.909 |
| 33 | D-Orn | H | (Gly)₅ | 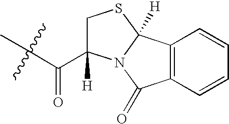 | 561.6 (M + 3, z = 3) C₇₇H₁₁₁N₂₁O₂₀S (z = 3): 561.6 |
| 34 | L-Lys | H | (Gly)₅ | 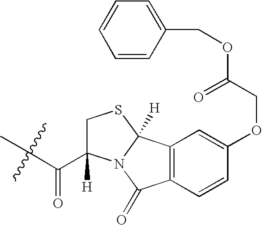 | 930.9183 (M + 2, z = 2); calc'd for C₈₇H₁₂₃N₂₁O₂₃S (z = 2): 930.9330 |
| 35 | D-Orn | H | (Gly)₅ | 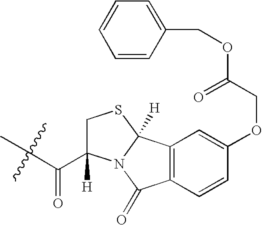 | 923.9175 (M + 2, z = 2); calc'd for C₈₆H₁₂₁N₂₁O₂₃S (z = 2): 923.9255 |
| 36 | L-Lys | 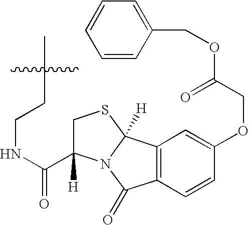 | Absent | H | 554.5; calc'd for C₈₂H₁₂₀N₂₂O₂₃S; 620.95 (M + 3H⁺, z = 3) |

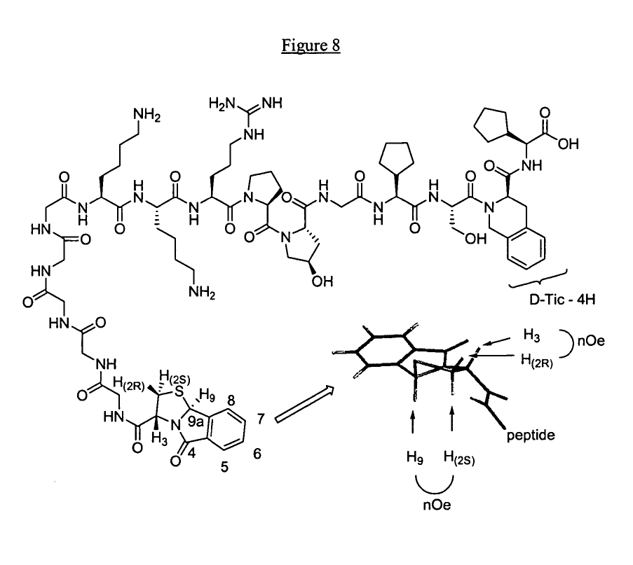

(3′R,9′bS)-3′-(carbonyl(HN-GGGGGKKRP(Hyp)G(Cpg)S(D-Tic)(Cpg)-OH))-2′,3′-dihydrothiazolo[2′,3′-a]isoindol-5′(9′bH)-one 32. Peptide 26 (116 mg, 72% peptide content, 52.8 μmol) was dissolved in 4.0 mL of 100 mM L-ascorbic acid/20 mM sodium-L-ascorbate. Methyl 2-formylbenzoate (10.4 mg, 63.3 μmol) was added followed by 400 μL MeOH. The reaction was stirred for 50 h. The solution was loaded onto column 2, and eluted with solvent system 2/gradient table 3 as defined in the Preparative Reverse Phase HPLC section of the general experimental. A band that eluted from 14-15 minutes was concentrated in vacuo to remove acetonitile, and lyophilized to afford the product. The peptide content by CLND was 56%. Selected ¹H NMR resonances for 26, assigned to protons shown in FIG. 8. ¹H NMR (400 MHz, DEUTERIUM OXIDE) δ ppm 4.96 (H₃, t, J=7.43 Hz, 1 H) 6.19 (H₉, s, 1 H) 7.15-7.28 (D-Tic, m, 4 H) 7.61 (H₆, t, J=7.43 Hz, 1 H) 7.64 (H₈, d, J=8.61 Hz, 1 H) 7.72 (H₇, t, J=7.04 Hz, 1 H) 7.79 (H₅, d J=7.82 Hz, 1 H). APCI MS (m/z) 848.8971 (M+2, z=2); calc'd for C₇₈H₁₁₅N₂₁O₂₀S (z=2): 848.909.

Peptides 33-36 were synthesized using the procedure described for 33. Mass spectral data is shown in table 7.

Detailed Structural Analysis for Peptide 32:

NMR Experiments.

Assignment of 1H NMR spectra were made from a combination of 2D Cosy45, 2D Noesy (phase sensitve, 25 and 40° C.), 2D ¹H/¹³C HSQC, 2D ¹H/¹³C HMBC at 600 MHz using a 5 mm inverse broadband probe. The stereochemical assignment for Hg was assigned relative to H₃, which is derived from L-cysteine.

Specifically, nOe (40° C.) was observed between H₉ and H_{(2S)}. The assignment of the geminal proton H_{(2R)} was obtained from the 2D Cosy45 experiment. This same resonance (H_{(2R)}) showed correlation to H₃ in the 40° C. 2D NOESY experiment. Taken together, the NMR experiments support the trans-relationship of H₉ and H₃ relative to the plane of the thiazoline ring (FIG. 8).

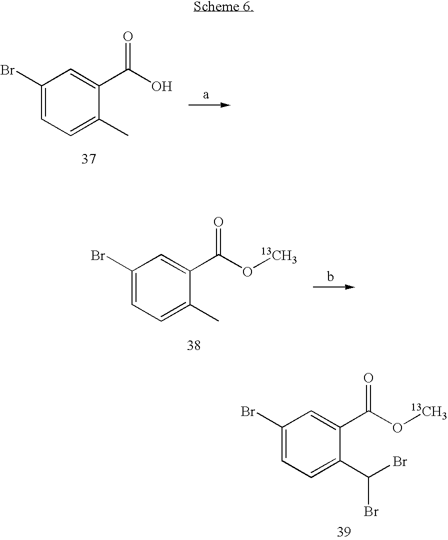

Reagents and Conditions: a) CDI, ¹³C-MeOH, DBU; b) NBS, AIBN.

¹³C Methyl 5-bromo-2-methyl benzoate (38). To a stirring solution of 5-Bromo-2-methyl benzoic acid (37) (25 g, 116 mmol) in 100 ml of dry DCM was added 1,1′-carbonyldiimidazole (21 g, 128 mmol). The solution was stirred for 3.5 h. The solution was transferred to a pressure vessel and treated with ¹³CH₃OH and DBU. The solution was washed with H₂O (2×20 mL), 5% NaHCO₃ (2×20 mL), and the organic layer was dried over MgSO₄. The solvent was removed in vacuo to yield the product. ¹H NMR (300 MHz, CHLOROFORM-d) δ ppm 2.59 (s, 3 H) 3.89 (d, J=147.12 Hz, 3 H) 7.39 (dd, J=8.29, 1.51 Hz, 1 H) 7.42 (s, 1 H) 7.79 (d, J=8.29 Hz, 1 H).

¹³C-Methyl 5-bromo-2-(dibromomethyl)benzoate (39). To a stirred solution of 38 (5.6 g 24 mmol) in CCl₄ was added N-bromosuccinimide (13.0 g, 73 mmol) and 2,2′-azobisisobutyronitrile (4.0 g, 24 mmol). The solution refluxed was refluxed until the starting material was consumed as judged by TLC. The mixture was purified by flash chromatography using a Biotage 40+ packed silica column with a gradient of 0-10% EtOAc/Hexane (R_{f} for 39=0.4 in 1:9 EtOAc/Hexane) to afford the title compound. ¹H NMR (300 MHz, CHLOROFORM-d) δ ppm 3.95 (d, J=147.91 Hz, 3 H) 7.52 (dd, J=8.48, 2.05 Hz, 1 H) 7.78 (d, J=8.48 Hz, 1 H) 8.00 (s, 1 H) 8.30 (d, J=1.90 Hz, 1 H)

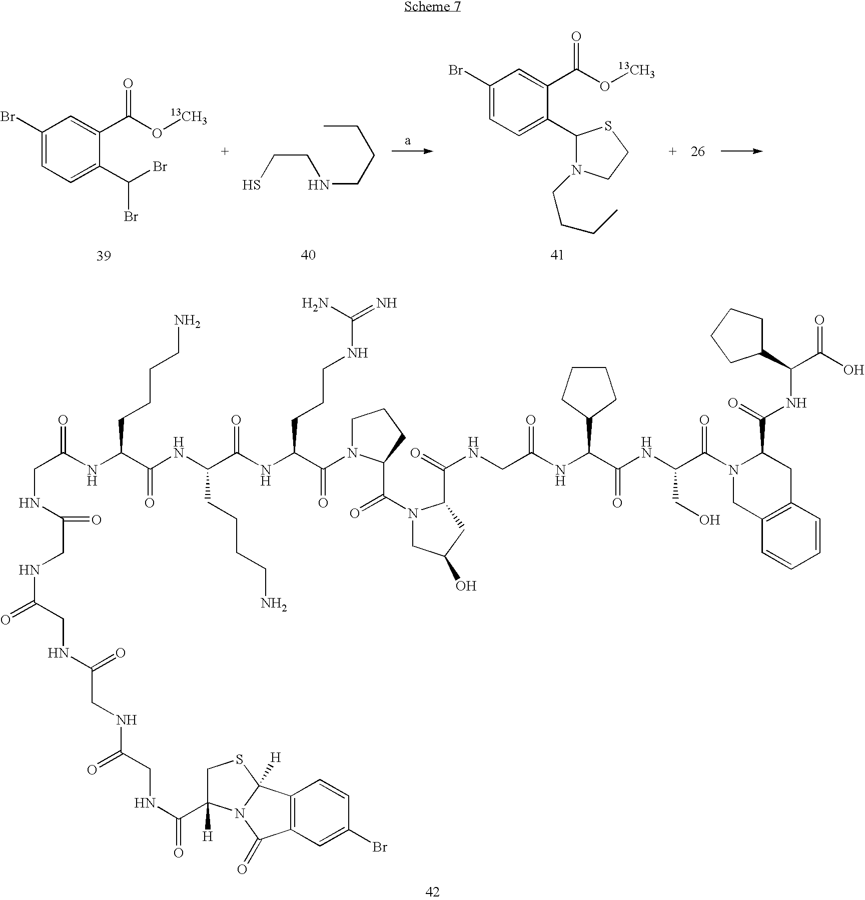

Reagents and Conditions: a) NaH, PS-DIEA; b) methanol-water, 100 mM L-ascorbic acid, 20 mM sodium-L-ascorbate.

¹³C-Methyl 5-bromo-2-(3-butylthiazolidin-2-yl)benzoate (41). To a stirred solution of 2-(butylamino)ethanethiol (40) (621.5 mg, 5 mmol) in 20 ml THF was added PS-triphenylphosphine (Argonaut Technologies, 2.1030 g, 5 mmol). The reaction stirred for 30 minutes and the solution was filtered using a medium porosity sintered glass funnel. The resin was agitated with THF (20 mL) by bubbling N₂ through the sintered glass for 10 min. The THF was filtered and combined with the first filtrate. The resin was washed a second time using the same protocol. The combined filtrates were cooled to 0° C. Sodium hydride (0.06 ml, 3 mmol), ¹³C-methyl 5-bromo-2-(dibromomethyl)benzoate (39) (897.0 mg, 2 mmol), and PS-DIEA (Argonaut Technologies, 1.2429 g, 5 mmol) was added and stirred at rt for 2 days. The reaction was refluxed overnight, cooled to rt and stirred for 10 days. The solution was filtered, concentrated in vacuo and purified by reverse phase chromatography (column 1, Solvent system 3, Gradient table 4). A band that eluted from 26 to 27 minutes was concentrated in vacuo to afford the title compound. APCI MS (m/z): 359.0 (M+H); Calc'd. for C₁₄³CH₂₁⁷⁹BrNO₂S: 359.04. APCI MS (m/z): 361.0(M+H); Calc'd for C₁₄¹³CH₂₁⁸¹BrNO₂S: 361.04. ¹H NMR (300 MHz, CHLOROFORM-d) δ ppm 0.91 (t, J=7.25 Hz, 3 H) 1.31-1.43 (m, J=11.30 Hz, 2 H) 1.46-1.59 (m, J=7.72 Hz, 2 H) 2.38-2.69 (m, J=12.06 Hz, 2 H) 2.85-3.01 (m, J=6.03 Hz, 2 H) 3.07-3.27 (m, J=11.21, 6.12 Hz, 2 H) 3.91 (d, J=147.50 Hz, 2 H) 5.88 (s, 1 H) 7.41 (dd, J=8.29, 1.88 Hz, 1 H) 7.68 (d, J=8.29 Hz, 1 H) 8.01 (s, 1 H).

(3′R,9′bS)-7′-Bromo-3′-(carbonyl(HN-GGGGGKKRP(Hyp)G(Cpg)S(D-Tic)(Cpg)-OH))-2′,3′-dihydrothiazolo[2′,3′-a]isoindol-5′(9′bH)-one 42. Prepared using the same procedure as described for 32. HR FTMS (m/z): 887.8612 (M+2, z=2); calc'd for C₇₈H₁₁₄⁷⁹BrN₂₁O₂₀S (z=2): 887.8650; 888.8509 (M+2, z=2); calc'd for C₇₈H₁₁₄⁸¹BrN₂₁O₂₀S (z=2): 888.8650.

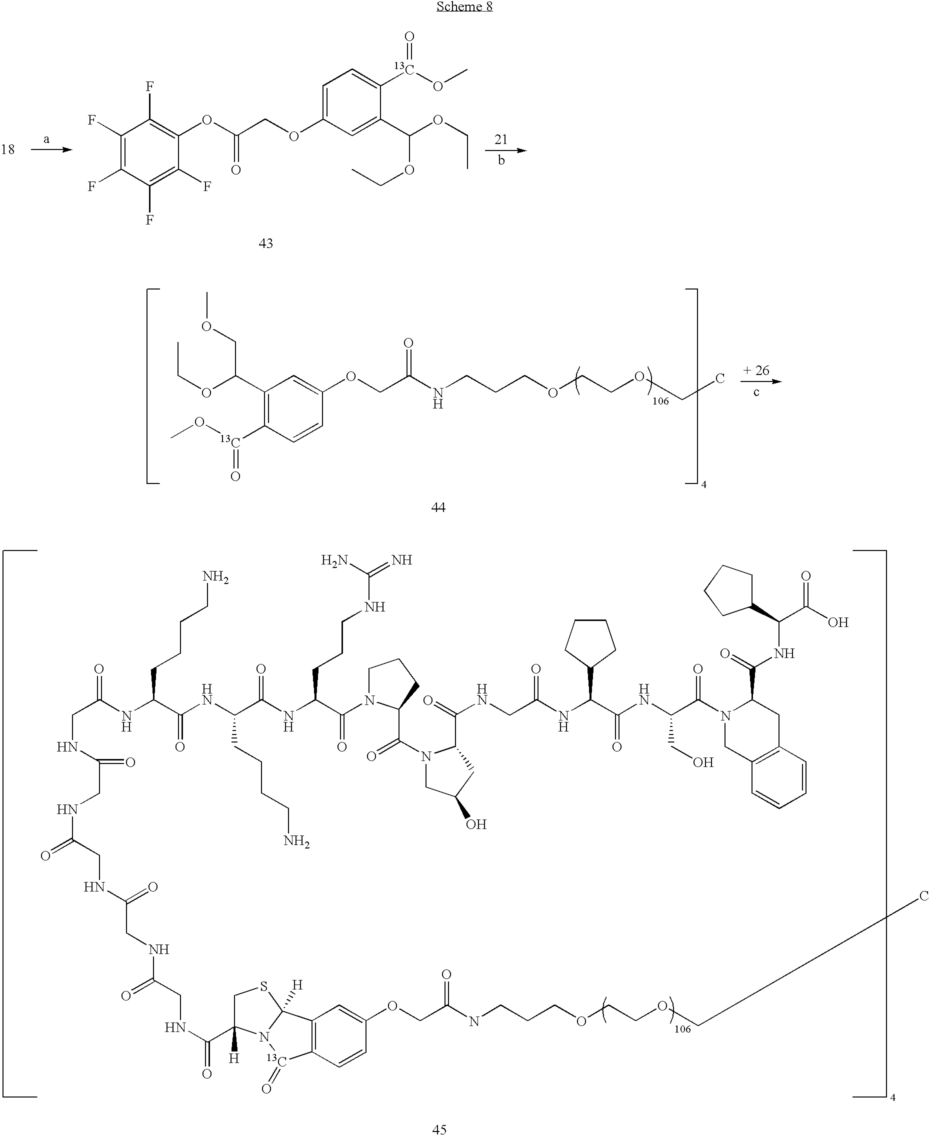

Reagents and conditions: a) PS-carbodiimide, pentafluoro phenol; b) PEG reagent 21, Hünig's base; c) D₂O, 100 mM LiCl, 50 mM deuterated ascorbic acid basified to pD 3.7 with 1M NaOD in D₂O.

Methyl 2-(diethoxymethyl)-4-(2-oxo-2-pentafluorophenoxyethoxy)benzoate (43).

To a 50 mL RB flask vacuum evacuated and backfilled with N₂ was added 132 mg of washed/dried 10% Pd on carbon (0.12 mmol Pd) and 4 mL anhydrous THF. The mixture was degassed by three cycles of careful evacuation (attempt to minimize bumping) and backfilling with N₂. A solution of Methyl 4-(2-(benzyloxy)-2-oxoethoxy)-2-(diethoxymethyl)benzoate (0.500g, 1.24 mmol) (17) in anhydrous THF (3 mL) was added to the slurry, the vial was washed with additional 1 mL THF and transferred to RB flask and degassed with three cycles of evacuation/nitrogen refill. Following last evacuation, H₂ from a balloon was used to backfill the vacuum evacuated RB flask. The reaction was stirred at rt under H₂ for 4 hr, at which time GC/MS (Method 3) indicated the reaction was complete, (17, 17.4 min, m/z=358.1, calc'd for C₁₉¹³CH₂₁O₆⁺=358.1, M-OEt; 18,14.26 min, m/z=268.1, calc'd for C₁₂¹³CH₁₅O₆⁺=268.1, M-OEt). The solution was filtered through a celite pad using a fine fritted glass vacuum filter into a 50 mL RB flask containing PS-carbodiimide (Argonaut Technologies, Inc, 2.4 g, 3.1 mmol) suspended in 15 mL anhydrous THF. The celite was washed with three portions of THF (3 mL), which were cobined with the filtrate/PS-carbodimide. The heterogenous mixture was stirred under N₂ for 20 min, and treated with pentafluorophenol (456 mg, 2.48 mmol) in THF. The reaction mixture was stirred under N₂ for 16 hr, at which time the reaction was complete by GC/MS (Method 3) (18, 14.26 min; 43, 15.6 min, m/z=434.1, calc'd for C₁₈¹³CF₅H₁₅O₆+=434.1, M-OEt). The mixture was filtered through a medium fritted funnel into a tarred 50 mL RB flask. 10 mL of THF was then added to the resin and mixed by gentle agitation using N₂ The filtrates were combined, the solvent was removed and product dried in vacuo to afford the product. EI MS m/z=434.1, calc'd for C₁₈¹³CF₅H₁₅O₆⁺=434.1, M-OEt)

Compound 44. To a 50 mL RB flask vacuum evacuated and backfilled with N₂ was added 20K tetraamino PEG (21, 440 mg, 22 μmol) and 3 mL anhydrous acetonitrile. The mixture was degassed by three cycles of careful evacuation (attempt to minimize bumping) and backfilling with N₂. To the solution was added Hünigs base (0.172 mmol, 30 μL) followed by a solution of methyl 2-(diethoxymethyl)-4-(2-oxo-2-pentafluorophenoxyethoxy)benzoate (43, 0.128 mmol) in anhydrous acetonitrile (1mL+1 mL for rinse). Molecular sieves (powdered, 4 Å pore, 100 mg) were added to the mixture. The solution was degassed with three cycles of evacuation/nitrogen refill. The reaction was stirred at 40° C. under N₂ for 24 hr. The mixture was filtered through a medium fritted funnel into a 50 mL RB flask containing Si-bound piperazine (Silicycle Inc., 171 mg, 0.15 mmol) and Si-bound carbonate (Silicycle Inc., 0.3 mmol, 434 mg) and washed with additional 10 mL of acetonitrile. The combined filtrates were stirred at 40° C. under N₂ for 15 hr. The mixture was then filtered through a medium fritted funnel into a tarred 50 mL RB flask, washed with additional 10 mL of acetonitrile. The solvent was removed and product dried in vacuo to afford 44. ¹³C NMR (D₂O, partial structure): δ 170.14, 72.00.

Compound 45 was synthesized as described for 28. The reaction was run at pD 3.7 in D₂O. Specifically a solution of 100 mM LiCl and 50 mM deuterated ascorbic acid (obtained by lyophilization from D₂O, three cycles) was prepared in D₂O. The pD was adjusted with 1 M NaOD to 3.7. To this solutipn was added PEG reagent 44 and peptide 26. The reaction was stiorred at rt for 13 h and worked up as described for 28. Structure by FT-MSMS was as similar to 28, but shifted by 1 amu higher due to the ¹³C.

### EXAMPLE

### In vivo Antinociceptive Activity of Polymer-Conjugated Anti-B1 Peptides in Rat and Monkey Pain Models

A. Rat Neuropathic Pain Model. Male Sprague-Dawley rats (200 g) are anesthetized with isoflurane inhalant anesthesia and the left lumbar spinal nerves at the level of L5 and L6 are tightly ligated (4-0 silk suture) distal to the dorsal root ganglion and prior to entrance into the sciatic nerve, as first described by Kim and Chung (An experimental model for peripheral neuropathy produced by segmental spinal nerve ligation in the rat. Pain 50:355-363, (1992)). The incisions are closed and the rats are allowed to recover. This procedure results in mechanical (tactile) allodynia in the left hind paw as assessed by recording the pressure at which the affected paw (ipsilateral to the site of nerve injury) is withdrawn from graded stimuli (von Frey filaments ranging from 4.0 to 148.1 mN) applied perpendicularly to the plantar surface of the paw (between the footpads) through wire-mesh observation cages. A paw withdrawal threshold (PWT) is determined by sequentially increasing and decreasing the stimulus strength and analyzing withdrawal data using a Dixon non-parametric test, as described by Chaplan, S. R., et al. (Quantitative assessment of tactile allodynia in the rat paw. J. Neurosci. Meth, 53:55-63 (1994)).

Normal rats and sham surgery rats (nerves isolated but not ligated) withstand at least 148.1 mN (equivalent to 15 g) of pressure without responding. Spinal nerve ligated rats respond to as little as 4.0 mN (equivalent to 0.41 g) of pressure on the affected paw. Rats may be included in the study only if they do not exhibit motor dysfunction (e.g., paw dragging or dropping) and their PWT was below 39.2 mN (equivalent to 4.0 g). At least seven days after surgery rats are treated with test peptides or test vehicle-conjugated peptides (usually a screening dose of about 1 mg/kg and about 60 mg/kg, respectively) or control diluent (PBS) once by s.c. injection and PWT is determined each day thereafter for 7 days.

B. Rat CFA Inflammatory Pain Model. Male Sprague-Dawley rats (200 g) are lightly anesthetized with isoflurane inhalant anesthesia and the left hindpaw is injected with complete Freund's adjuvant (CFA), 0.15 ml. This procedure results in mechanical (tactile) allodynia in the left hind paw as assessed by recording the pressure at which the affected paw is withdrawn from graded stimuli (von Frey filaments ranging from 4.0 to 148.1 mN) applied perpendicularly to the plantar surface of the paw (between the footpads) through wire-mesh observation cages. PWT is determined by sequentially increasing and decreasing the stimulus strength and analyzing withdrawal data using a Dixon non-parametric test, as described by Chaplan et al. (1994). Rats should be included in the study only if they do not exhibit motor dysfunction (e.g., paw dragging or dropping) or broken skin and their PWT is below 39.2 mN (equivalent to 4.0 g). At least seven days after CFA injection rats can be treated with test polymer-conjugated peptides (usually a screening dose of around 60 mg/kg) or control solution (PBS) once by s.c. injection and PWT may be determined each day thereafter for 7 days. Average paw withdrawal threshold (PWT) can be converted to percent of maximum possible effect (% MPE) using the following formula: % MPE=100* (PWT of treated rats−PWT of control rats)/(15-PWT of control rats). Thus, the cutoff value of 15 g (148.1 mN) is equivalent to 100% of the MPE and the control response is equivalent to 0% MPE.

Preferred polymer-conjugated peptides of the present invention are expected to produce an antinociceptive effect with a PD relationship at a screening dose of about 1 mg/kg and about 60 mg/kg, respectively.

B. Green Monkey LPS Inflammation Model. The effectiveness of polymer conjugated peptides as inhibitors of B1 activity may be evaluated in Male green monkeys (Cercopithaecus aethiops St Kitts) challenged locally with B1 agonists essentially as described by deBlois and Horlick (British Journal of Pharmacology. 132:327-335 (2002)), which is hereby incorporated by reference in its entirety).

In order to determine whether PEG-conjugated peptide antagonists of the present invention inhibit B1 induced oedema the studies described below may be conducted on male green monkeys (Cercopithaecus aethiops St Kitts; Caribbean Primates Ltd. experimental farm (St Kitts, West Indies)). Animals weighing 6.0±0.5 kg (n=67) are anaesthetized (50 mg ketamine kg⁻¹) and pretreated with a single intravenous injection of LPS (90 μg kg⁻¹) or saline (1 ml) via the saphenous vein.

1. Inflammation Studies

Kinin-induced oedema may be evaluated by the ventral skin fold assay (Sciberras et al., 1987). Briefly, anaesthetized monkeys are injected with captopril (1 mg kg⁻¹ 30 min before assay). A single subcutaneous injection of dKD, BK or the vehicle (2 mM amastatin in 100 μl Ringer's lactate) is given in the ventral area and the increase in thickness of skin folds is monitored for 30-45 min using a calibrated caliper. The results can be expressed as the difference between the skin fold thickness before and after the subcutaneous injection. Captopril and amastatin may beused to reduce degradation of kinins at the carboxyl- and amino-terminus, respectively.

Antagonist Schild Analysis

The dose-response relationship for dKD (1-100 nmol)-induced oedema can be determined at 24 h post-LPS in the absence or presence of different concentrations of PEG-peptide antagonist. BK (30 nmol) may be used as a positive control.

Antagonst Time Course

The time course of inhibition by antagonist can be determined at 4, 24, 48, 72 and/or 96 h after single bolus administration. BK (30 nmol) may be used as a positive control.

Drugs

Ketamine hydrochloride, LPS, amastatin and captopril may be purchased from Sigma (Mo., U.S.A.). All peptides can be obtained from Phoenix Pharmaceuticals (Calif., U.S.A.).

Statistics

Values can be presented as mean±standard error of the mean (s.e. mean). In edema studies, the pre-injection thickness of the skin folds is subtracted from the values after subcutaneous challenge. Curve fitting and EC₅₀ calculations may be obtained using the Delta Graph 4.0 software for Apple Computers. Data are compared by two-way analysis of variance followed by unpaired, one tail Student's t-test with Bonferroni correction. p<0.05 is considered statistically significant.

LPS administration to green monkeys should increase from a null level their sensitivity to a B₁ receptor agonist in an edema formation assay. Comparatively, responses to the B₂ receptor agonist BK should not be affected.

### EXAMPLE

### Rat Pharmacokinetic Studies

Various peptides or conjugated peptides (in an aqueous medium) are dosed as a bolus to male Sprague-Dawley rats via an intravenous (iv) or subcutaneous (sc) route. Blood samples are collected at various time points (e.g., 0, 15, 30 min. and/or 1, 2, 4, 6, 8, 10, 12, 18, 24, 30, 36, 42, 48, 60, 72, 84, 96, 120, 240, and/or 320 hours after the injection) into heparized tubes. Plasma is removed from pelleted cells upon centrifugation and either frozen or immediately processed. The compound of interest in the plasma is quantitated by an analyte-specific LC-MS/MS or an ELISA method. Various standard pharmacokinetic parameters such as clearance (CL), apparent clearance (CL/F), volume of distribution (Vss), mean residence time (MRT), area under the curve (AUC), and terminal half-life (t_{1/2}) may be calculated by non-compartmental method.

What is claimed is:

1. A compound having the structure: 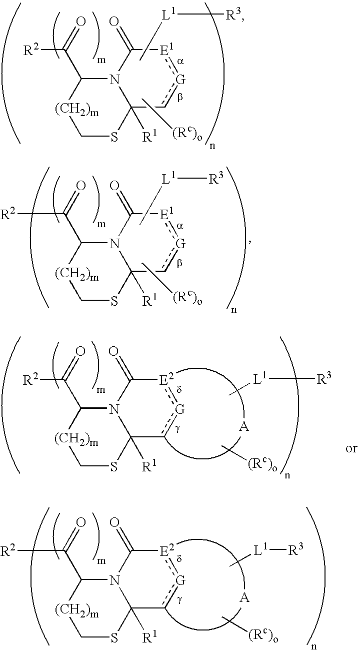
or a pharmaceutically acceptable salt or hydrate thereof, wherein:
A is a saturated, partially-saturated, or unsaturated 2-, 3-, 4-, 5- or 6-atom bridge containing 0, 1, 2, or 3 heteroatoms selected from O, N, and S, with the remaining bridge atoms being carbon;
E¹ is N, O, or C;
E² is N or C;
G is a single bond, a double bond, C, N, O, B, S, Si, P, Se, or Te; 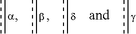
are each a single bond and one of 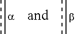
may additionally be a double bond; and when G is C or N one of 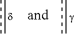
may additionally be a double bond; and when G is a singlelebond or a double bond, 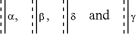
all absent;
L¹ is a divalent C₁₋₆alkyl or C₁₋₆heteroalkyl, both of which are substituted by 0, 1, 2, or 3 substituents selected from F, Cl, Br, I, OR^{a}, NR^{a}R^{a} and oxo;
m is independently in each instance, 0 or 1;
n is greater than or equal to 1;
o is 0, 1, 2, 3, 4 or 5;
R¹ is H, C₁₋₆alkyl, phenyl or benzyl, any of which is substituted by 0, 1, 2, or 3 groups selected from halo, cyano, nitro, oxo, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{a}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from F, Br, Cl and I;
R² is a vehicle and R³ a bioactive compound; or R³ is a vehicle and R² a bioactive compound;
R^{a} is independently, at each instance, H or R^{b};
R^{b} is independently, at each instance, phenyl, benzyl or C₁₋₆alkyl, the phenyl, benzyl and C₁₋₆alkyl being substituted by 0, 1, 2, or 3 substituents selected from halo, C₁₋₄alkyl, C₁₋₃haloalkyl, —OC₁₋₄alkyl, OH, —NH₂, —NHC₁₋₄alkyl, and —N(C₁₋₄alkyl)C₁₋₄alkyl; and
R^{c} is independently, in each instance, selected from halo, C₁₋₆-alkyl, C₁₋₃haloalkyl, —OC₁₋₄alkyl, OH, —NH₂, —NHC₁₋₄alkyl and —N(C₁₋₄alkyl)C₁₋₄alkyl.

2. A compound according to claim 1 having the general structure:

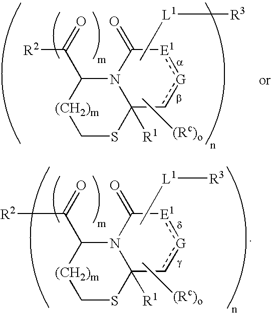

3. A compound according to claim 1 having the general structure:

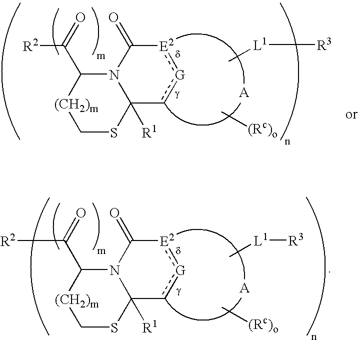

4. A compound according to claim 3, wherein A is a saturated, partially-saturated, or unsaturated 2-, 3-, 4-, 5- or 6-atom bridge containing 1, 2, or 3 heteroatoms selected from O, N, and S, with the remaining bridge atoms being carbon.

5. A compound according to claim 3, wherein A is a saturated, partially-saturated, or unsaturated 2-, 3-, 4-, 5- or 6-carbon-atom bridge.

6. A compound according to claim 3, wherein:
A is a an unsaturated 4-carbon-atom bridge;
E² is C; and
G is a double bond.

7. A compound according to claim 1, wherein G is a single bond or a double bond and 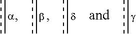
are all absent.

8. A compound according to claim 1, wherein G is C, N, O, B, S, Si, P, Se, or Te.

9. A compound according to claim 1, wherein 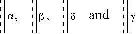
are each a single bond.

10. A compound according to claim 1, wherein:
G is C or N; and
one of 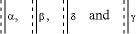
is a double bond.

11. A compound according to claim 1, wherein R² is a vehicle and R³ a bioactive compound.

12. A compound according to claim 1, wherein R³ is a vehicle and R² a bioactive compound.

13. The compound according to claim 1, wherein R³ selected from poly(alkylene oxide), poly(vinyl pyrrolidone), poly(vinyl alcohol), polyoxazoline, poly(acryloylmorpholine-), poly(oxyethylated polyol), poly(ethylene glycol), carboxymethylcellulose, dextran, polyvinyl alcohol, polyvinyl pyrrolidone, poly-1,3-dioxolane, poly-1,3,6-trioxane, an amino acid homopolymer, polypropylene oxide, a copolymer of ethylene glycol/propylene glycol, an ethylene/maleic anhydride copolymer, an amino acid copolymer, a copolymer of PEG and an amino acid, a polypropylene oxide/ethylene oxide copolymer, and a polyethylene glyco/thiomalic acid copolymer; or any combination thereof.

14. The compound according to claim 1, wherein R³ is PEG.

15. The compound according to claim 1, wherein R² is a B1 peptide antagonist.

16. The compound according to claim 1, wherein R² is a B1 peptide antagonist is a peptide selected from SEQ ID NOS:5-26 and 42-62 wherein said peptide was modified to have a N-terminal cysteine residue.

17. A method for preparing a compound according to claim 1, comprising the step of reacting:
R²—(C(═O))ₘCH(NH₂)CH₂(CH₂)ₘSH with A) 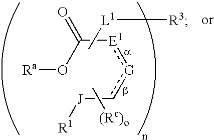
R²—[(C(═O))ₘCH(NH₂)CH₂(CH₂)ₘSH]ₙ with B) 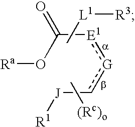
wherein J is a carbonyl or a protected version thereof.

18. A method for preparing a compound according to claim 1, comprising the step of reacting:
R²—(C(═O))ₘCH(NH₂)CH₂(CH₂)ₘSH with A) 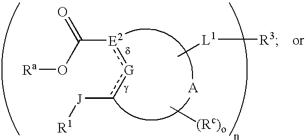
R²—[(C(═O))ₘCH(NH₂)CH₂(CH₂)ₘSH]ₙ with B) 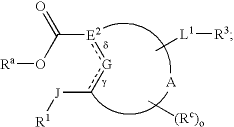
wherein J is a carbonyl or a protected version thereof.

19. A method according to claim 17, wherein J is selected from C(═O), C(OCH₂CH₂O), C(N(R^{a})CH₂CH₂N(R^{a})), C(N(R^{a})CH₂CH₂O), C(N(R^{a})CH₂CH₂S), C(OCH₂CH₂CH₂O), C(N(R^{a})CH₂CH₂CH₂N(R^{a})), C(N(R^{a})CH₂CH₂CH₂O), C(N(R^{a})CH₂CH₂CH₂S), C(OR^{b})₂, C(SR^{b})₂ and C(NR^{a}R^{b})₂.

20. A method according to claim 17, wherein the reaction is perfomed at a pH between 2 and 7.

21. A method according to claim 17, wherein the reaction is perfomed at a pH between 3 and 5.

22. A method according to claim 18, wherein J is selected from C(═O), C(OCH₂CH₂O), C(N(R^{a})CH₂CH₂N(R^{a})), C(N(R^{a})CH₂CH₂O), C(N(R^{a})CH₂CH₂S), C(OCH₂CH₂CH₂O), C(N(R^{a})CH₂CH₂CH₂N(R^{a})), C(N(R^{a})CH₂CH₂CH₂O), C(N(R^{a})CH₂CH₂CH₂S), C(OR^{b})₂, C(SR^{b})₂ and C(NR^{a}R^{b})₂.

23. A method according to claim 18, wherein the reaction is perfomed at a pH between 2 and 7.

24. A method according to claim 18, wherein the reaction is perfomed at a pH between 3 and 5.

25. A compound having the structure: 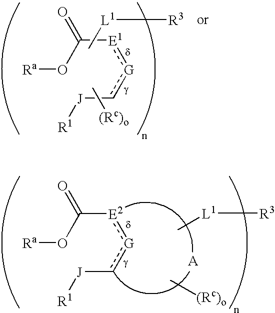
wherein:
A is a saturated, partially-saturated, or unsaturated 2-, 3-, 4-, 5- or 6-atom bridge containing 0, 1, 2, or 3 heteroatoms selected from O, N, and S, with the remaining bridge atoms being carbon;
E¹ is N, O, or C;
E² is N or C;
G is a single bond, a double bond, C, N, O, B, S, Si, P, Se, or Te; 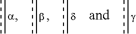
are each a single bond and one of 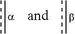
may additionally be a double bond; and when G is C or N one of 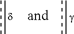
may additionally be a double bond; and when G is a single bond or a double bond, 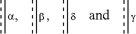
are all absent;
J is a carbonyl or a protected version thereof;
L¹ is a divalent C₁₋₁₂alkyl or C₁₋₁₂heteroalkyl, both of which are substituted by 0, 1, 2, or 3 substituents selected from F, Cl, Br, I, OR^{a}, NR^{a}R^{a} and oxo;
m is independently in each instance, 0 or 1;
n is 1, 2, 3, 4, 5, 6, 7, 8, 9 or 10;
o is 0, 1, 2, 3, 4 or 5;
R¹ is H, C₁₋₆alkyl, phenyl or benzyl, any of which is substituted by 0, 1, 2, or 3 groups selected from halo, cyano, nitro, oxo, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}and —NR^{a}C₂₋₆alkylOR^{a}, and additionally substituted by 0, 1, 2, 3, 4, 5 or 6 atoms selected from F, Br, Cl and I;
R³ is a bioactive compound or a vehicle;
R^{a}is independently, at each instance, H or R^{b};
R^{b} is independently, at each instance, phenyl, benzyl or C₁₋₆alkyl, the phenyl, benzyl and C₁₋₆alkyl being substituted by 0, 1, 2, or 3 substituents selected from halo, C₁₋₄alkyl, C₁₋₃haloalkyl, —OC₁₋₄alkyl, OH, —NH₂, —NHC₁₋₄alkyl, and —N(C₁₋₄alkyl)C₁₋₄alkyl;
R^{c} is independently, in each instance, selected from halo, C₁₋₄alkyl, C₁₋₃haloalkyl, —OC₁₋₄alkyl, OH, —NH₂, —NHC₁₋₄alkyl and —N(C₁₋₄alkyl)C₁₋₄alkyl; and
X is C(═O) and Y is NH; or X is NH and Y is C(═O).

26. A compound according to claim 25 having the general structure:

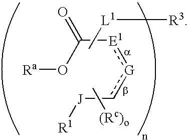

27. A compound according to claim 25 having the general structure:

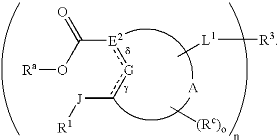

28. A compound according to claim 27, wherein A is a saturated, partially-saturated, or unsaturated 2-, 3-, 4-, 5- or 6-atom bridge containing 1, 2, or 3 heteroatoms selected from O, N, and S, with the remaining bridge atoms being carbon.

29. A compound according to claim 27, wherein A is a saturated, partially-saturated, or unsaturated 2-, 3-, 4-, 5- or 6-carbon-atom bridge.

30. A compound according to claim 27, wherein:
A is a an unsaturated 4-carbon-atom bridge;
E² is C; and
G is a double bond.

31. A compound according to claim 25, wherein G is a single bond or a double bond and 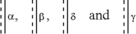
are all absent.

32. A compound according to claim 25, wherein G is C, N, O, B, S, Si, P, Se, or Te.

33. A compound according to claim 25, wherein 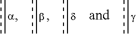
are each a single bond.

34. A compound according to claim 25, wherein:
G is C or N; and
one of 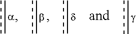
is a double bond.

35. A compound according to claim 25, wherein R³ a bioactive compound.

36. A compound according to claim 25, wherein R³ is a vehicle.

37. The compound according to claim 25, wherein R³ selected from poly(alkylene oxide), poly(vinyl pyrrolidone), poly(vinyl alcohol), polyoxazoline, poly(acryloylmorpholine-), poly(oxyethylated polyol), poly(ethylene glycol), carboxymethylcellulose, dextran, polyvinyl alcohol, polyvinyl pyrrolidone, poly-1,3-dioxolane, poly-1,3,6-trioxane, an amino acid homopolymer, polypropylene oxide, a copolymer of ethylene glycol/propylene glycol, an ethylene/maleic anhydride copolymer, an amino acid copolymer, a copolymer of PEG and an amino acid, a polypropylene oxide/ethylene oxide copolymer, and a polyethylene glyco/thiomalic acid copolymer; or any combination thereof.

38. The compound according to claim 25, wherein R³ is PEG.

39. A method for preparing a compound according to claim 25, comprising the step of reacting (Y-L²)ₙ-R³ with 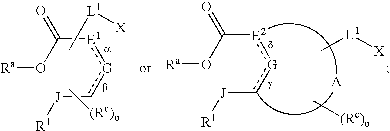
L² is independently, in each instance C₁₋₆-alkyl or C₁₋₆heteroalkyl both of which are substituted by 0, 1, 2, 3 or 4 substituents selected from F, Cl, Br, I, OR^{a}, NR^{a}R^{a} and oxo;
X is a nucleophile and Y is an electrophile; or X is an electrophile and Y is a nucleophile.

40. A method accordingly claim 39, wherein:
the nucleophile is selected from SH, NH₂ and OH; and
the electrophile is selected from CH₂halogen, CH₂SO₂OR^{b} C(═O)O(succinimide), C(═O)O(perfluoroalkyl), C(═O)O(CH₂CN) and C(═O)O(C₆F₅).

41. A method of treating pain and/or inflammation comprising the administration to a patient in need thereof of a therapeutically-effective amount of a compound according to claim 1.

42. A pharmaceutical composition comprising a compound according to claim 1 and a pharmaceutically acceptable carrier or dilluent.

43. The manufacture of a medicament comprising a compound according to claim 1.
